(19) United States
(12) Patent Application Publication
(10) Pub. No.: US 2008/0311037 A1
(43) Pub. Date: Dec. 18, 2008

(54) Compounds which bind PSMA and uses thereof

(76) Inventors: Warren D.W. Heston, Chagrin Falls, OH (US); Hagen Cramer, West Chester, OH (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/897,539

(22) Filed: Aug. 30, 2007

Related U.S. Application Data
(63) Continuation of application No. PCT/US06/07141, filed on Mar. 1, 2006.
(60) Provisional application No. 60/671,996, filed on Apr. 15, 2005.
(60) Provisional application No. 60/658,005, filed on Mar. 2, 2005.
(60) Provisional application No. 60/660,941, filed on Mar. 11, 2005.

(51) Int. Cl. C07H 19/20; C07H 21/00; A61K 31/7088; A61K 31/7076; C12Q 1/02; A61K 49/00; G01N 33/574; C07F 5/00; A61K 51/04; A61P 9/00; A61P 25/00; A61P 35/00

(52) U.S. Cl. 424/1.85; 536/26.6; 536/25.6; 514/47; 514/44; 435/29; 424/9.1; 435/7.23; 534/15; 424/1.65; 424/1.81

(57) ABSTRACT

A compound is represented by Structural Formula A1:

C—B-L-A A1
or a pharmaceutically acceptable salt or solvate thereof.
A is a prostate specific membrane antigen (PSMA) ligand;
L is an optionally substituted aliphatic or heteroaliphatic linking group;
B includes at least one optionally substituted moiety selected from the group consisting of a sugar, a charged group, an aryl ring, and a heteroaryl ring, wherein B optionally includes a drug or a labeling agent; and
C is H, a drug, or a labeling agent, wherein CB together comprises the drug or the labeling agent.

The compounds are useful as PSMA agents and in pharmaceutical compositions, methods for treating and detecting diseases such as cancer in a subject, methods for identifying cancer cells in a sample, methods for inhibiting tumor neovascularization, methods for identifying drugs that can treat cancer, and the like.

### RELATED APPLICATIONS

This application is a continuation of International Application No. PCT/US2006/007141, which designated the United States and was filed on Mar. 1, 2006, published in English, which claims the benefit of U.S. Provisional Application No. 60/671,996, filed on Apr. 15, 2005; U.S. Provisional Application No. 60/658,005, filed on Mar. 2, 2005; and U.S. Provisional Application No. 60/660,941, filed on Mar. 11, 2005.

The entire teachings of the above applications are incorporated herein by reference.

### GOVERNMENT SUPPORT

The invention was supported, in part, by grant # CA103943-1 from the National Cancer Institute and by a grant CA 101069-02 from National Institutes of Health (NIH). The Government has certain rights in the invention.

### BACKGROUND OF THE INVENTION

Prostate specific membrane antigen (PSMA) is a protein belonging to the enzyme family of glutamate carboxypeptidase IIs also named GCP2 or CPG2. Besides the prostate, GCP2s can be found in significant quantities in the brain as N-acetylated α-linked acidic dipeptidase (NAALADase). PSMA is also expressed on the tumor vascular endothelium of virtually all solid (NAALADase). PSMA is also expressed on the tumor vascular endothelium of virtually all solid carcinomas and sarcomas but not on normal vascular endothelium. Thus, it is desirable to target GCP2s for imaging and for therapy for various associated diseases.

Antibodies have been developed to target GCP2s such as PSMA. However, at least one example is known to bind to an intracellular portion of PSMA and thus likely to be imaging dead prostate cells, and is known to have issues of sensitivity and specificity. Moreover, antibody agents can take a long time to equilibrate and diffuse into tumors, and can have high non-specific binding to macrophages, leading to non-specific retention in critical tissues such as the liver.

Low molecular weight, non-antibody ligands have been developed that bind to PSMA and NAALADase. However, the ligands only have limited ability to penetrate the blood-brain barrier, which does not favor their use as neuroprotective agents.

Therefore, there is a need for compounds which target GCP2s, including PSMA, for use in therapy and in imaging.

### SUMMARY OF THE INVENTION

Novel compounds that are PSMA agents, pharmaceutical compositions comprising these compounds, methods for treating and detecting cancers in a subject, methods for identifying cancer cells in a sample, methods for identifying drugs that can treat cancer or inhibit tumor neovascularization, and the like are disclosed herein.

A compound is represented by Structural Formula A1:

C—B-L-A A1

or a pharmaceutically acceptable salt or solvate thereof.

A is a prostate specific membrane antigen (PSMA) ligand;

L is an optionally substituted aliphatic or heteroaliphatic linking group;

B includes at least one optionally substituted moiety selected from the group consisting of a sugar, a charged group, an aryl ring, a heteroaryl ring, and a nucleobase, wherein B optionally includes a drug or a labeling agent; and

C is H, a drug, or a labeling agent, wherein CB together comprises the drug or the labeling agent.

In some embodiments, C is H and B comprises the drug or the labeling agent. In some embodiments, B includes at least two optionally substituted moieties selected from the group consisting of a sugar, a charged group, an aryl ring, a heteroaryl ring, and a nucleobase.

In various embodiments the drug or the labeling agent is coupled to the rest of the compound by a cleavable linker.

In various embodiments, when A includes HO₂CCH₂CH₂CH(CO₂H)CH₂—P(O)(OH)— or HO₂CCH₂CH₂CH(CO₂H)CH₂—OP(O)(OH)—, CB does not include:

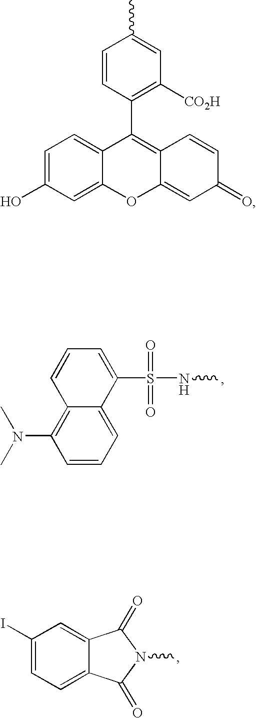

or unsubstituted phenyl;

when A1 includes a moiety represented by either of the following structural formulas:

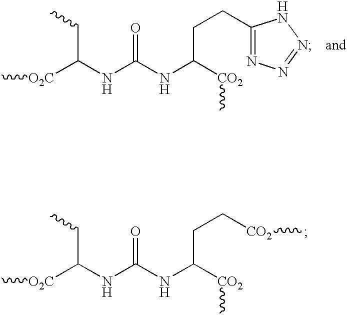

then CB includes at least one group selected from: a covalently attached, nonmetallic charged group other than carboxylate or protonated amine; a sugar; and a heteroaryl or non-aromatic heterocycle having at least two heteroatoms;

when A1 includes a moiety represented by the following structural formula:

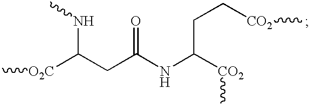

then CB includes at least one group selected from: a covalently attached, nonmetallic charged group other than carboxylate, protonated amine, or sulfate; a sugar; and a heteroaryl or non-aromatic heterocycle having at least two heteroatoms; and

when A1 includes a moiety represented by the following structural formula:

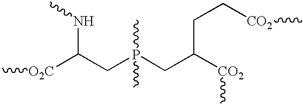

then CB includes at least one group selected from: a covalently attached, nonmetallic charged group other than carboxylate or protonated amine; a sugar other than an aminosaccharide; and a heteroaryl or non-aromatic heterocycle.

In some embodiments, the compound is represented by Structural Formula A2:

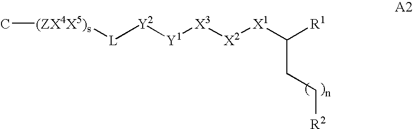

n is 0, 1, or 2;

R¹ and R² are independently carboxylate or carboxylate bioisosteres;

X¹, X², X³ and X⁴ are independently a bond, —NR^{a}—, —O—, —S—, —CR^{a}R^{b}—, —CR^{b}(OR^{a})—, —CR^{b}(SR^{a})—, —C(O)—, —C(S)—, —C(═CR^{a}R^{b})—, —C(═NR^{a})—, —C(═NOR^{a})—, —C(═NNR^{a})—, —S(O)—, —(SO₂)—, —S(O)(R^{a})—, —S(O)(OR^{a})—, —(PO₂)—, —P(O)(R^{a})—, —P(O)(OR^{a})—, —OP(O)(R^{a})—, —OP(O)(OR^{a})—, —P(S)(R^{a})—, —P(S)(OR^{a})—, —OP(S)(R^{a})—, or —OP(S)(OR^{a})—;

Y¹ and Y² are a bond, or Y¹ is an optionally substituted C1-C6 aliphatic chain and Y² is O or S;

L is an optionally substituted aliphatic or heteroaliphatic linking group;

s is an integer from 1 to 6, wherein the variables in each (ZX⁴X⁵) are independently selected, e.g., each (ZX⁴X⁵) can be the same or different;

each Z is independently an optionally substituted aryl, heteroaryl, cycloaliphatic, or non-aromatic heterocyclic group, provided that at least one Z comprises an aryl, heteroaryl, nucleobase, nucleoside, or nucleotide;

X⁵ is a bond or methylene;
C-(ZX⁴X⁵)s- comprises a drug or a labeling agent; and

R^{a} and R^{b} are each independently —H or an optionally substituted aliphatic, optionally substituted cycloaliphatic, optionally substituted heterocyclic, optionally substituted benzyl, optionally substituted aryl, or optionally substituted heteroaryl.

In some embodiments, the compound is represented by Structural Formula A3:

C-(ZX⁴X⁵)-L-A A3

wherein the variables are as provided above or further in the Detailed Description below.

A pharmaceutical composition includes the compound and a pharmaceutically acceptable carrier. In various embodiments, the pharmaceutical composition includes a drug, e.g., an anticancer drug; a labeling agent, e.g., a fluorescent labeling agent, a radionuclide, and the like. In some embodiments, the pharmaceutically acceptable carrier can include delivery systems known to the art for encapsulating drugs.

A method of treating cancer includes administering the compound to a subject in need thereof, wherein the compound includes a drug, e.g., an anticancer drug.

A method of inhibiting tumor neovascularization, includes administering the compound to a subject in need thereof, wherein the compound includes a drug that inhibits neovascularization, e.g., an anticancer drug.

A method of treating a disease mediated by neovascularization includes administering the compound to a subject in need thereof, wherein the compound includes a drug that inhibits neovascularization, e.g., an anticancer drug. In various embodiments, diseases dependent on neovascularization that can be treated by inhibition thereof include rheumatoid arthritis, macular degeneration, and the like.

A method of treating a neurological disease includes administering the compound to a subject in need thereof, wherein the compound includes a drug that inhibits neovascularization, e.g., an anticancer drug. In various embodiments, diseases dependent on neovascularization that can be treated by inhibition thereof include rheumatoid arthritis, macular degeneration, and the like.

A method of identifying a drug to treat cancer includes contacting a cell which expresses prostate specific membrane antigen with the compound, wherein the compound includes a drug to be assessed, and determining whether the compound has a therapeutic effect on the cell. If the compound has a therapeutic effect on the cell, then the compound can be used to treat cancer. Such therapeutic effects on the cell can include one or more of killing the cell, rendering the cell quiescent, inducing differentiation of the cell, or inhibiting the cell's ability to mestasize. The cell can be obtained from a solid tumor, for example, from the neovasculature of a solid tumor.

A method of identifying a drug to treat prostate cancer includes contacting a prostate cancer cell with the compound, and determining whether the compound has a therapeutic effect on the prostate cancer cell, wherein the compound includes a drug to be assessed.

A method of identifying a drug that inhibits tumor neovascularization includes contacting a tumor neovasculature cell which expresses prostate specific membrane antigen with the compound, and determining whether the compound has a therapeutic effect on the cell, wherein the compound includes a drug.

A method of detecting cancer in a subject includes administering the compound to a subject, wherein the compound includes a labeling agent, and detecting the labeling agent in the subject. In various embodiments, the subject has a solid tumor comprising prostate specific membrane antigen in its neovasculature; the subject has prostate cancer; the subject is at risk of cancer; the cancer is detected before a biopsy is conducted; recurrence of the cancer after cancer therapy is detected; metastasis of the cancer is detected; early stage cancer is detected; or the distribution of the labeling agent in the subject is detected as a two dimensional or three dimensional image, optionally as a function of time.

A method of identifying cancer cells in a sample includes contacting the sample with the compound, wherein the compound includes a labeling agent, and detecting the labeling agent. In various embodiments, the sample is obtained from a source selected from the group consisting of blood, plasma, serum, cerebrospinal fluid, urine, kidney ultrafiltrate, gastrointestinal contents, gall bladder contents, ovarian fluid, seminal fluid, amniotic fluid, tumor ascites and other tumor fluids, expressed prostatic secretions, bone marrow aspirates, or from computed tomography or magnetic resonance imaging. In various embodiments, the sample includes cells from a solid tumor; cells from the neovasculature of a tumor; or prostate cancer cells. In some embodiments, the distribution of the labeling agent is detected as a two dimensional or three dimensional image, optionally as a function of time.

A kit includes the compound, wherein the compound includes a drug or labeling agent, and can include instructions for employing the compound.

The compounds, pharmaceutical compositions, kits, and methods herein are believed to be effective for treating and detecting cancers in a subject, identifying cancer cells in a sample, identifying drugs that can treat cancer or inhibit tumor neovascularization, and the like.

In various embodiments, the disclosed compounds and methods can be used to treat subjects (e.g., humans) with neurological disorders including, for example, stroke, neuropathy (e.g., diabetic/insulin induced neuropathy, or drug-induced neuropathy, e.g., peripheral neuropathy induced by anticancer agents such etoposide) pain, neuropathic pain, epilepsy, trauma (head trauma, spinal cord trauma), ischemia, amyotrophic lateral sclerosis (ALS), schizophrenia, Huntington's disease, Parkinson's disease, cocaine addiction, epilepsy, demyelinating diseases, inflammation, and Alzheimer's disease.

### BRIEF DESCRIPTION OF THE DRAWINGS

FIG. 1A is a bar graph showing percentage of radiolabeled compound bound versus concentration where the disclosed 2-5A ligand binds to PSMA at lower concentrations (EC₅₀=1.5 nM) compared to compound (ZJ24) (the S-methylated analog of parent PSMA ligand (6)) (EC₅₀=15.3 nM) or the intermediate 2-5A-SMCC oligonucleotide-linker (26) (RBI 1032) (which can be used to probe for nonspecific binding).

FIG. 1B is a plot of activity versus log(concentration) for the three compounds, where the disclosed 2-5A ligand inhibits PSMA at lower concentrations (EC₅₀=0.62 nM) compared to compound (ZJ24) (the S-methylated analog of parent PSMA ligand (6)) (EC₅₀=56.7 nM) or the intermediate 2-5A-SMCC oligonucleotide-linker (26) (RBI 1032).

FIG. 2 is a graph showing binding to the monomeric and active dimeric form of soluble recombinant hPSMA of a ³H (tritium) radiolabled (ZJ24), ³H—(S)-2-[3-((R)-1-carboxy-2-methyl-sulfanyl-ethyl)-ureido]-pentanedioic acid or [³H]MeCys-C(O)-Glu.

FIG. 3 shows PSMA receptor-mediated internalization of 2-5A ligands.

### DETAILED DESCRIPTION OF THE INVENTION

A description of preferred embodiments of the invention follows.

### Compounds

In various embodiments of the compounds of the present invention the variables in Structural Formulas A1, A2, and A3 are as defined above or as further provided in this section (COMPOUNDS).

Without wishing to be bound by theory, it is believed that binding of the claimed compounds can be due to the PSMA ligand (e.g., represented by variable A in Structural Formula A1) in combination with B, wherein B includes at least one optionally substituted moiety selected from the group consisting of a sugar, a charged group, an aryl ring, and a heteroaryl ring. Generally, B includes at least two of these optionally substituted moieties, for example, a sugar and a charged group; a sugar and an aryl ring; a sugar and a heteroaryl ring; an aryl ring and a charged group; a heteroaryl ring and a charged group; an aryl ring and a heteroaryl ring; or the like. Typically, B includes at least three of these optionally substituted moieties, for example a sugar, a charged group, and an aryl ring; a sugar, a charged group, and a heteroaryl ring; or the like. More typically, B includes an optionally substituted sugar, a charged group, and a heteroaryl ring.

In various embodiments, the compound is represented by Structural Formula A4:

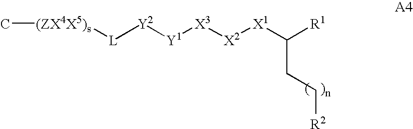

n is 0, 1, or 2;

R¹ and R² are independently carboxylate or carboxylate bioisosteres;

X¹, X², X³ and X⁴ are independently a bond, —NR^{a}—, —O—, —S—, —CR^{a}R^{b}—, —CR^{b}(OR^{a})—, —CR^{b}(SR^{a})—, —C(O)—, —C(S)—, —C(═CR^{a}R^{b})—, —C(═NR^{a})—, —C(═NOR^{a})—, —C(═NNR^{a})—, —S(O)—, —(SO₂)—, —S(O)(R^{a})—, —S(O)(OR^{a})—, —(PO₂)—, —P(O)(R^{a})—, —P(O)(OR^{a})—, —OP(O)(R^{a})—, —OP(O)(OR^{a})—, —P(S)(R^{a})—, —P(S)(OR^{a})—, —OP(S)(R^{a})—, or —OP(S)(OR^{a})—;

Y¹ and Y² are a bond, or Y¹ is an optionally substituted C1-C6 aliphatic chain and Y² is O or S;

L is an optionally substituted aliphatic or heteroaliphatic linking group;

s is an integer from 1 to 6, wherein the variables in each (ZX⁴X⁵) are independently selected, e.g., (ZX⁴X⁵) can be the same or different;

each Z is independently an optionally substituted aryl, heteroaryl, cycloaliphatic, or non-aromatic heterocyclic group, provided that at least one Z is an aryl or heteroaryl group or is substituted with an aryl or heteroaryl group; and

X⁵ is a bond or methylene,

wherein R^{a} and R^{b} are each independently —H or an optionally substituted aliphatic, optionally substituted cycloaliphatic, optionally substituted heterocyclic, optionally substituted benzyl, optionally substituted aryl, or optionally substituted heteroaryl.

In various embodiments, the compound is represented by Structural Formula A5:

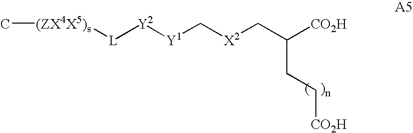

x² can be —(PO₂)—, —P(O)(R^{a})—, —P(O)(OR^{a})—, —OP(O)(R^{a})—, —OP(O)(OR^{a})—, —P(S)(R^{a})—, —P(S)(OR^{a})—, —OP(S)(R^{a})—, or —OP(S)(OR^{a})—.

In some embodiments, the compound can be represented by Structural Formula A6:

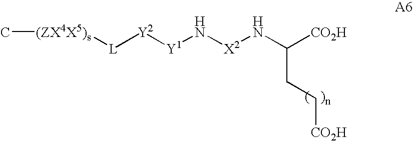

or typically, by Structural Formula A7 wherein X⁶ is O or S:

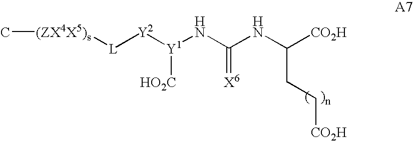

or more typically, by Structural Formula A8:

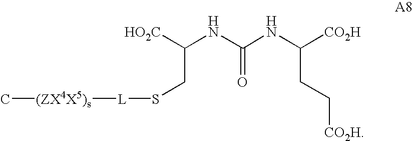

In some embodiments, at least one ZX⁴X⁵ (e.g., in any of Structural Formulas A2-A8) can include an optionally substituted nucleobase such as optionally substituted adenine, guanine, cytosine, thymine, uracil, and the like, typically adenine or guanine, more typically adenine. An optionally substituted nucleobase can be optionally substituted with any suitable optional substituent described in the “Definitions” of this section. Moreover, a substituted nucleobase can include the corresponding ribonucleosides, deoxyribonucleosides, ribonucleotides, and deoxyribonucleotides each of which may be further optionally substituted.

In some embodiments, the compound can be represented by Structural Formula A9:

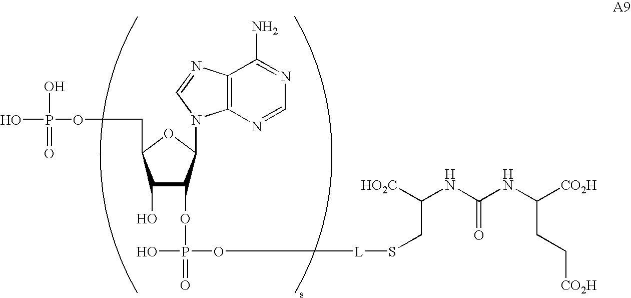

or more typically, in various embodiments, the compound can be represented by Structural Formula A10:

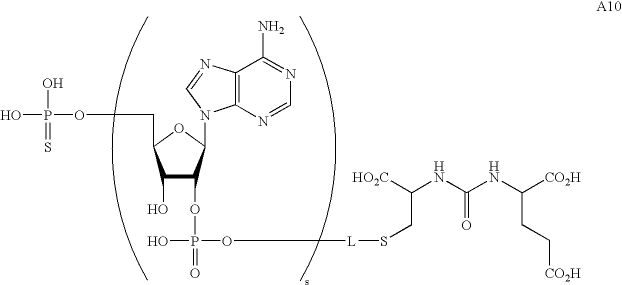

wherein compared to Structural Formula A9, certain phosphate oxygen atoms are replaced with sulfur.

In some embodiments, the compound is represented by Structural Formula 11:

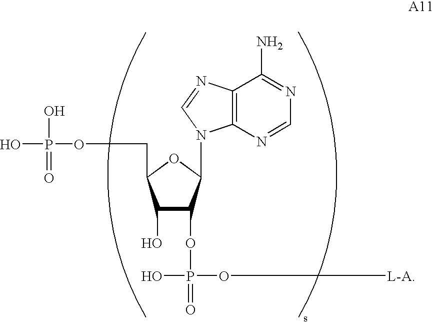

or more typically, by Structural Formula A12:

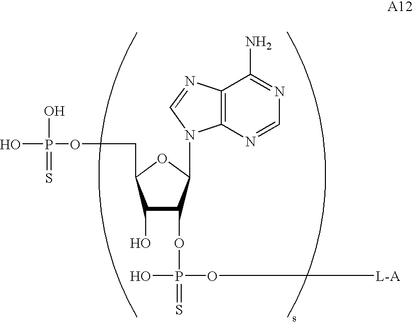

wherein compared to Structural Formula A11, certain phosphate oxygen atoms are replaced with sulfur.

In various embodiments, L (e.g., in any of Structural Formulas A1-A12) can include at least one ring selected from an optionally substituted 4 to 7 membered nonaromatic heterocyclic ring and an optionally substituted C4-C7 cycloalkyl ring.

In a preferred embodiment, the compound is represented by Structural Formula A13:

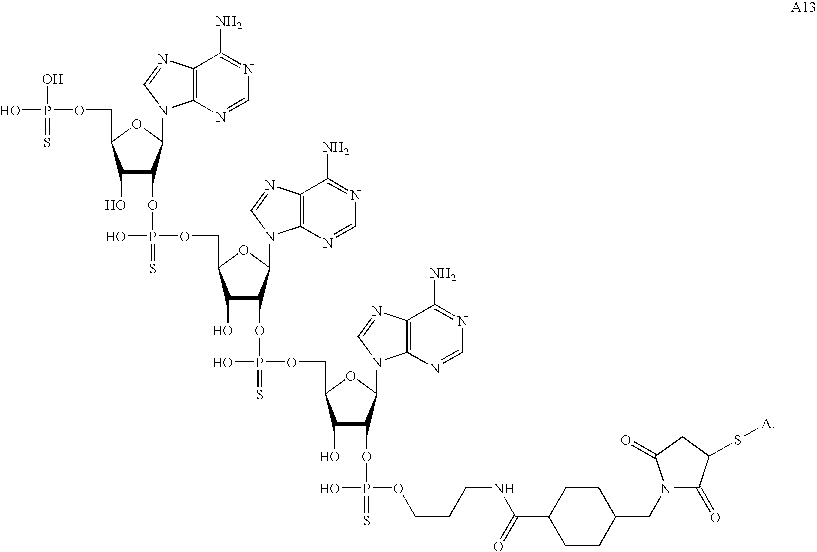

In a preferred embodiment, the compound is represented by Structural Formula A14:

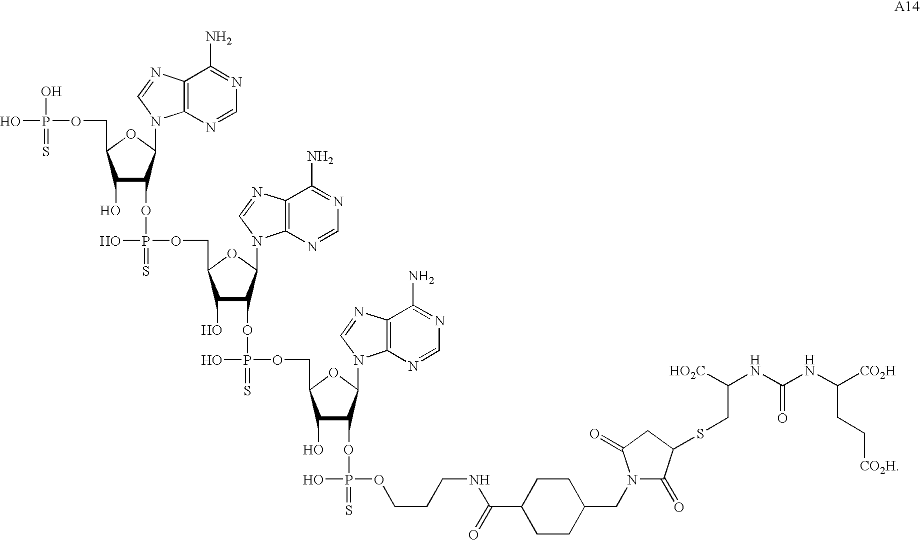

An aliphatic group is a straight chained, branched or cyclic non-aromatic hydrocarbon which is completely saturated or which contains one or more units of unsaturation. An alkyl group is a saturated aliphatic group. Typically, a straight chained or branched aliphatic group has from 1 to about 10 carbon atoms, preferably from 1 to about 4, and a cyclic aliphatic group has from 3 to about 10 carbon atoms, preferably from 3 to about 8. An aliphatic group is preferably a straight chained or branched alkyl group, e.g., methyl, ethyl, n-propyl, iso-propyl, n-butyl, sec-butyl, tert-butyl, pentyl, hexyl, pentyl or octyl, or a cycloalkyl group with 3 to about 8 carbon atoms. C1-C4 straight chained or branched alkyl or alkoxy groups or a C3-C8 cyclic alkyl or alkoxy group (preferably C1-C4 straight chained or branched alkyl or alkoxy group) are also referred to as a “lower alkyl” or “lower alkoxy” groups; such groups substituted with —F, —Cl, —Br, or —I are “lower haloalkyl” or “lower haloalkoxy” groups; a “lower hydroxyalkyl” is a lower alkyl substituted with —OH; and the like.

An “alkylene group” is represented by —(CH₂)ₙ—, wherein n is an integer from 1-10, preferably 1-4.

The term “aryl” (for example the aryl groups that can be included in CB) refers to C6-C14 carbocyclic aromatic groups such as phenyl, biphenyl, and the like. Aryl groups also include fused polycyclic aromatic ring systems in which a carbocyclic aromatic ring is fused to other aryl, cycloalkyl, or cycloaliphatic rings, such as naphthyl, pyrenyl, anthracyl, and the like.

The term “heteroaryl” refers to 5-14 membered heteroaryl groups having 1 or more O, S, or N heteroatoms. Examples of heteroaryl groups include imidazolyl, isoimidazolyl, thienyl, furanyl, fluorenyl, pyridyl, pyrimidyl, pyranyl, pyrazolyl, pyrrolyl, pyrazinyl, thiazoyl, isothiazolyl, oxazolyl, isooxazolyl, 1,2,3-trizaolyl, 1,2,4-triazolyl, imidazolyl, thienyl, pyrimidinyl, quinazolinyl, indolyl, tetrazolyl, and the like. Heteroaryl groups also include fused polycyclic aromatic ring systems in which a carbocyclic aromatic ring or heteroaryl ring is fused to one or more other heteroaryl rings. Examples include benzothienyl, benzofuranyl, indolyl, quinolinyl, benzothiazolyl, benzoisothiazolyl, benzooxazolyl, benzoisooxazolyl, benzimidazolyl, quinolinyl, isoquinolinyl, isoindolyl, and the like.

The term heteroaryl (for example the heteroaryl groups that can be included in CB) can also refer to the various nucleobases, e.g., 9H-purin-6-amine (adenine), 2-amino-1H-purin-6(9H)-one (guanine), 4-aminopyrimidin-2(1H)-one (cytosine), pyrimidine-2,4(1H,3H)-dione (uracil), and 5-methylpyrimidine-2,4(1H,3H)-dione (thymine).

The term “sugar” (for example the sugar that can be included in CB) includes monosaccharides such as trioses, tetroses, pentoses, hexoses, heptoses, octoses and nonoses. For example, hexoses can include allose, altrose, glucose, mannose, gulose, idose, galactose, and talose; and pentoses can include fructose, ribose, and deoxyribose; and the like. Also included are polymers thereof, e.g., disaccharides, trisaccharides oligosaccharides, and polysaccharides.

Heterocyclic groups are non-aromatic carbocyclic rings which include one or more heteroatoms such as N, O, or S in the ring. The ring can be five, six, seven or eight-membered. Examples include oxazolinyl, thiazolinyl, oxazolidinyl, thiazolidinyl, tetrahydrofuranyl, tetrahyrothiophenyl, morpholino, thiomorpholino, pyrrolidinyl, piperazinyl, piperidinyl, thiazolidinyl, and the like.

Suitable optional substituents for a substitutable atom in alkyl, cycloalkyl, aliphatic, cycloaliphatic, heterocyclic, benzylic, aryl, or heteroaryl groups are those substituents that do not substantially interfere with the activity of the disclosed compounds. A “substitutable atom” is an atom that has one or more valences or charges available to form one or more corresponding covalent or ionic bonds with a substituent. For example, a carbon atom with one valence available (e.g., —C(—H)═) can form a single bond to an alkyl group (e.g., —C(-alkyl)=), a carbon atom with two valences available (e.g., —C(H₂)—) can form one or two single bonds to one or two substituents (e.g., —C(alkyl)(Br))—, —C(alkyl)(H)—) or a double bond to one substituent (e.g., —C(═O)—), and the like. Substitutions contemplated herein include only those substitutions that form stable compounds.

For example, suitable optional substituents for substitutable carbon atoms include —F, —Cl, —Br, —I, —CN, —NO₂, —OR^{a}, —C(O)R^{a}, —OC(O)R^{a}, —C(O)OR^{a}, —SR^{a}, —C(S)R^{a}, —OC(S)R^{a}, —C(S)OR^{a}, —C(O)SR^{a}, —C(S)SR^{a}, —S(O)R^{a}, —SO₂R^{a}, —SO₃R^{a}, —POR^{a}R^{b}, PO₂R^{a}R^{b}, —PO₃R^{a}R^{b}, —PO₄R^{a}R^{b}, —P(S)R^{a}R^{b}, —P(S)OR^{a}R^{b}, —P(S)O₂R^{a}R^{b}, —P(S)O₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a}, —NR^{c}C(O)N(R^{a}R^{b}), —C(NR^{c})—N(R^{a}R^{b}), —NR^{d}—C(NR^{c})—N(R^{a}R^{b}), —NR^{a}N(R^{a}R^{b}), —CRC═CR^{a}R^{b}, —C═CR^{a}, ═O, ═S, ═CR^{a}R^{b}, ═NR^{a}, ═NOR^{a}, ═NNR^{a}, optionally substituted alkyl, optionally substituted cycloalkyl, optionally substituted aliphatic, optionally substituted cycloaliphatic, optionally substituted heterocyclic, optionally substituted benzyl, optionally substituted aryl, and optionally substituted heteroaryl, wherein R^{a}—R^{d} are each independently —H or an optionally substituted aliphatic, optionally substituted cycloaliphatic, optionally substituted heterocyclic, optionally substituted benzyl, optionally substituted aryl, or optionally substituted heteroaryl, or, —N(R^{a}R^{b}), taken together, is an optionally substituted heterocyclic group. Also contemplated are isomers of these groups.

Suitable substituents for nitrogen atoms having two covalent bonds to other atoms include, for example, optionally substituted alkyl, optionally substituted cycloalkyl, optionally substituted aliphatic, optionally substituted cycloaliphatic, optionally substituted heterocyclic, optionally substituted benzyl, optionally substituted aryl, optionally substituted heteroaryl, —CN, —NO₂, —OR^{a}, —C(O)R^{a}, —OC(O)R^{a}, —C(O)OR^{a}, —SR^{a}, —S(O)R^{a}, —SO₂R^{a}, —SO₃R^{a}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a}, —NR^{c}C(O)N(R^{a}R^{b}), and the like.

A nitrogen-containing heteroaryl or non-aromatic heterocycle can be substituted with oxygen to form an N-oxide, e.g., as in a pyridyl N-oxide, piperidyl N-oxide, and the like. For example, in various embodiments, a ring nitrogen atom in a nitrogen-containing heterocyclic or heteroaryl group can be substituted to form an N-oxide.

Suitable substituents for nitrogen atoms having three covalent bonds to other atoms include —OH, alkyl, and alkoxy (preferably C1-C4 alkyl and alkoxy). Substituted ring nitrogen atoms that have three covalent bonds to other ring atoms are positively charged, which is balanced by counteranions such as chloride, bromide, fluoride, iodide, formate, acetate and the like. Examples of other suitable counteranions are provided in the section below directed to suitable pharmacologically acceptable salts.

Typically, the disclosed compounds have at least one substituent that is a carboxylic acid derivative or a bioisostere thereof. As used herein, “isosteres” refer to elements, functional groups, substituents, molecules or ions having different molecular formulae but exhibiting similar or identical physical properties. Typically, two isosteric molecules have one or more similarities in their volume, shape, charge or charge distribution, polarizability, ionizability, and the like. Typically, isosteric compounds can be isomorphic and can co-crystallize. Other physical properties that can be similar among isosteric compounds include boiling point, density, viscosity and thermal conductivity. However, not all properties need be identical; certain properties can be different such as dipolar moment, polarity, polarization, volume, shape, and the like. The term “isosteres” encompasses “bioisosteres” which are isosteres that, in addition to their physical similarities, share one or more common biological properties. For example, tetrazole is a bioisostere of carboxylic acid because it can mimic some properties of a carboxylic acid group even though it has a different molecular formula. Typically, bioisosteres interact with the same recognition site or can produce broadly similar biological effects. See, for example, Wermuth, CG “Molecular Variations Based on Isosteric Replacements” pp 203-238, in The Practice of Medicinal Chemistry, Wermuth, CG ed, Academic Press, New York, 2^{nd} Ed, 1996; the entire teachings of which are incorporated herein by reference.

Thus “carboxylic acid bioisosteres” include, for example, direct derivatives such as hydroxamic acids, acyl-cyanamides, and acylsulfonamides; planar acidic heterocycles such as tetrazoles, mercaptoazoles, sulfinylazoles, sulfonylazoles, isoxazoles, isothiazoles, hydroxythiadiazoles, and hydroxychromes (e.g., tetrazole, 1,2,3-triazole, 1,2,4-triazole and imidazole); sulfur- or phosphorus-derived acidic functions such as phosphinates, phosphonates, phosphonamides, sulphonates, sulphonamides, acylsulphonamides, alkylsulfonylcarbamoyl, arylsulfonylcarbamoyl and heteroarylsulfonylcarbamoyl; and the like.

In various embodiments, a group that is a carboxylic acid derivative or bioisostere thereof can be —OH, —CN, —NO₂, —C(O)R^{a}, —OC(O)R^{a}, —C(O)OR^{a}, —C(S)R^{a}, —OC(S)R^{a}, —C(S)OR^{a}, —C(O)SR^{a}, —C(S)SR^{a}, —S(O)R^{a}, —SO₂R^{a}, —SO₃R^{a}, —PO₂R^{a}R^{b}, —PO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —SO₂N(R^{a}R^{b}), —SO₂N(R^{a}R^{b}), —NR^{c}C(O)R^{a}, —NR^{c}C(O)OR^{a}, —NR^{c}C(O)N(R^{a}R^{b}), —C(NR^{c})—N(R^{a}R^{b}), —NR^{d}—C(NR^{c})—N(R^{a}R^{b}), —NR^{a}N(R^{a}R^{b}), ═NR^{a}, ═NOR^{a}, ═NNR^{a}, or optionally substituted tetrazole, mercaptoazole, sulfinylazole, sulfonylazole, isoxazole, isothiazole, hydroxythiadiazole, or hydroxychrome. Generally, a group that is a carboxylic acid derivative or bioisostere thereof can be —OH, —OC(O)R^{a}, —C(O)OR^{a}, —C(S)OR^{a}, —C(O)SR^{a}, —C(S)SR^{a}, —S(O)R^{a}, —SO₂R^{a}, —SO₃R^{a}, —PO₂R^{a}R^{b}, —PO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —SO₂N(R^{a}R^{b}), —SO₂N(R^{a}R^{b}), —NR^{c}C(O)R^{a}, —NR^{c}C(O)OR^{a}, —NR^{c}C(O)N(R^{a}R^{b}), ═NR^{a}, or optionally substituted tetrazole, 1,2,3-triazole, 1,2,4-triazole or imidazole. Typically, a group that is a carboxylic acid derivative or bioisostere thereof can be —OH, -aryl-OH, (e.g., -phenol), —C(O)OH, —C(S)OH, —C(O)SH, —C(S)SH, —SO₂H, —SO₃H, —PO₂H₂, —PO₃H₂, —NHR^{a}, —NH—, —C(O)NHR^{a}, —C(O)NHNHSO₂R^{c}, —C(O)NHSO₂R^{c}, —SO₂NHR^{a}, —SO₂NHR^{a}, —NHC(O)R^{a}, —NHC(O)OR^{a}, —NHC(O)NHR^{a}, NH, or optionally substituted tetrazole, 1,2,3-triazole, 1,2,4-triazole or imidazole. More typically, a group that is a carboxylic acid derivative or bioisostere thereof is -aryl-OH (e.g., -phenol), —OH, —CO₂H, —NHC(O)CH₃, —NHC(O)OCH₃, —NHC(O)OCH₃, —NH—, ═NH, tetrazole, 1,2,3-triazole, 1,2,4-triazole or imidazole. Preferably, a group that is a carboxylic acid derivative or bioisostere thereof is -phenol, —CO₂H, —NHC(O)CH₃, —NHC(O)OCH₃, —NHC(O)OCH₃, or an amine.

As used herein, a “charged group” (for example the charged group that can be included in CB) is a group that can carry a charge or be ionized under physiological conditions as part of an ionic bond, as a deprotonated acid, as a protonated base, and the like. For example, charged groups can be or can be generated from carboxylates, phosphates, phosphonates, amines, sulfates, sulfonates, or other of the optional substitutents and bioisosteres above that are ionizable or have ionic bonds under physiological conditions.

The disclosed compounds and methods can be used to treat subjects (e.g., humans) with cancer. As used herein, “cancer” includes human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, stomach cancer, esophageal cancer, uterine cancer, brain cancer, testicular tumor, lung carcinoma, small cell lung carcinoma, bladder carcinoma, epithelial carcinoma, gliomas, astrocytoma, medulloblastoma, craniopharyngioma, ependymoma, pinealoma, hemangioblastoma, acoustic neuroma, oligodendroglioma, meningioma, melanomas, neuroblastoma, retinoblastoma; and polycythemia vera, lymphoma (Hodgkin's disease and non-Hodgkin's disease), multiple myeloma, Waldenstrobm's macroglobulinemia, and heavy chain disease. Other examples of cancers are described in The Chemotherapy Sourcebook, Michael C. Perry Ed., Williams & Williams (1992) and Holland Frie Cancer Medicine 5th Ed., Bast et al. Eds., B.C. Decker Inc. (2000). The entire teachings of the preceding references are incorporated herein by reference. Typically, the cancer that can be treated is a solid tumor. In some embodiments, the cancer is selected from sarcomas, gliomas, melanomas, colon carcinoma, pancreatic cancer, breast cancer, ovarian cancer, prostate cancer, cervical cancer, stomach cancer, esophageal cancer, uterine cancer, brain cancer, testicular tumor, lung carcinoma, small cell lung carcinoma, bladder carcinoma, epithelial carcinoma, renal cell carcinoma, hepatoma, lung carcinoma, small cell lung carcinoma, or bladder carcinoma.

In various embodiments, the disclosed compounds and methods can be used to treat subjects (e.g., humans) with disorders including, for example, stroke, neuropathy (e.g., diabetic/insulin induced neuropathy, peripheral neuropathy (e.g., caused by anticancer agents such as etoposide) pain, neuropathic pain, epilepsy, trauma (head trauma, spinal cord trauma, nerve trauma, sciatic nerve trauma) amyotrophic lateral sclerosis (ALS), schizophrenia, Huntington's disease, Parkinson's disease, cocaine addiction, epilepsy, demyelinating diseases, glaucoma, inflammation, and Alzheimer's disease.

For example, the compounds can be administered to treat peripheral neuropathy, pain, neuropathic pain, spinal cord trauma, nerve trauma, sciatic nerve trauma, amyotrophic lateral sclerosis (ALS), glaucoma, and inflammation.

Also, the disclosed compounds and methods can be used to treat subjects (e.g., humans) with neurological disorders. As used herein, neurological disorders include stroke, epilepsy, trauma (head trauma), ischemia, amyotrophic lateral sclerosis (ALS), schizophrenia, Huntington's disease, Parkinson's disease, cocaine addiction, epilepsy, demyelinating diseases, brain inflammation, and Alzheimer's disease.

In various embodiments, the disclosed compounds and methods can be used to provide subjects with cognition enhancement.

In some embodiments, the disclosed compounds can be modified to cross the blood brain barrier. For example, the disclosed compounds can be employed in conjuction with biodegradable microspheres or coated cationic liposomes (CCLs), which have been employed to help oligonucleotides cross the blood brain barrier. See Brignole, C., Pagnan, G., Marimpietri, D., Cosimo, E., Allen, T. M., Ponzoni, M., and Pastorino, F. (2003), “Targeted delivery system for antisense oligonucleotides: a novel experimental strategy for neuroblastoma treatment.” Cancer Lett., 197, 231-5, Khan, A., Sommer, W., Fuxe, K., and Akhtar, S. (2000), “Site-specific administration of antisense oligonucleotides using biodegradable polymer microspheres provides sustained delivery and improved subcellular biodistribution in the neostriatum of the rat brain.” J. Drug Target, 8, 319-34). In some embodiments, blood-brain-barrier disruption (BBBD) or local delivery methods such as convection-enhanced delivery (CED) can be employed to deliver the disclosed compounds to brain targets See, for example, Hall, W. A., Rustamzadeh, E., and Asher, A. L. (2003). “Convection-enhanced delivery in clinical trials.” Neurosurg. Focus, 14, e2. The entire teachings of each reference cited in this paragraph are incorporated herein by reference.

The disclosed compounds can be co-administered with or can include (for example, as the drug that can be included by CB) other therapeutic agents, for example, anticancer agents such as Taxol, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin.

Other anti-cancer agents can include the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Arnad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-IAABE (Cytoskeleton/Mt. Sinai School of Medicine), A-204197 (Abbott), T-607 (Tularik, also known as T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCl), Resverastatin phosphate sodium, BPR-OY-007 (National Health Research Institutes), and SSR-250411 (Sanofi).

Examples of other agents include therapeutic antibodies e.g., HERCEPTIN® (Trastuzumab) (Genentech, Calif.) which is a humanized anti-HER2 monoclonal antibody for the treatment of patients with metastatic breast cancer; REOPRO® (abciximab) (Centocor) which is an anti-glycoprotein IIb/IIIa receptor on the platelets for the prevention of clot formation; ZENAPAX® (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/MedImmune); Campath 1H/LDP-03 which is a humanized anti CD52 IgGl antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); LYMPHOCIDE™ Y-90 (Immunomedics); Lymphoscan (Tc-99m-labeled; radioimaging; Immunomedics); Nuvion (against CD3; Protein Design Labs); CM3 is a humanized anti-ICAM3 antibody (ICOS Pharm); IDEC-114 is a primatied anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-α antibody (CAT/BASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CD20-sreptdavidin (+biotin-yttrium 90; NeoRx); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-β2 antibody (Cambridge Ab Tech).

Other agents that can be used include chemotherapeutic agents such as alkylating agents, antimetabolites, natural products, or hormones. Examples of alkylating agents useful for the treatment or prevention of T-cell malignancies in the methods and compositions of the invention include but are not limited to, nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites useful for the treatment or prevention of T-cell malignancies in the methods and compositions of the invention include but are not limited to folic acid analog (e.g., methotrexate), or pyrimidine analogs (e.g., Cytarabine), purine analogs (e.g., mercaptopurine, thioguanine, pentostatin). Examples of natural products useful for the treatment or prevention of T-cell malignancies in the methods and compositions of the invention include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents useful for the treatment or prevention of cancer in the methods and compositions of the invention include but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites useful for the treatment or prevention of cancer in the methods and compositions of the invention include but are not limited to folic acid analog (e.g., methotrexate), or pyrimidine analogs (e.g., fluorouracil, floxouridine, Cytarabine), purine analogs (e.g., mercaptopurine, thioguanine, pentostatin). Examples of natural products useful for the treatment or prevention of cancer in the methods and compositions of the invention include but are not limited to vinca alkaloids (e.g., vinblastin, vincristine), epipodophyllotoxins (e.g., etoposide, teniposide), antibiotics (e.g., actinomycin D, daunorubicin, doxorubicin, bleomycin, plicamycin, mitomycin), enzymes (e.g., L-asparaginase), or biological response modifiers (e.g., interferon alpha). Examples of hormones and antagonists useful for the treatment or prevention of cancer in the methods and compositions of the invention include but are not limited to adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and compositions of the invention for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, amino glutethimide).

In various embodiments, the compound includes a labeling agent (for example, the labeling agent that can be included by CB) selected from the group consisting of fluorescent labeling agents, quantum dots, magnetic resonance imaging (MRI) contrast agents, and radionuclides. Suitable examples of such agents are those well known to the art.

For example, suitable radionuclides can include atomic isotopes such as ^{99m}Tc, ¹¹¹In, ¹²³I, ¹³¹I, ⁶⁷Ga, ²⁰¹Tl, ¹²⁵I, ¹⁸F, ¹¹C, ⁷⁶Br, ¹²⁴I, ⁶⁸Ga, ⁸²R^{b}, ¹³N, ⁶⁴Cu, ⁹⁰Y, ¹⁸⁸Rh, T(tritium), ³²P, ³⁵S, ¹⁵³Sm, ⁸⁹Sr, and ²¹¹At. These isotopes can be incorporated into the disclosed compound by methods known to the art, for example, as labeled nucleotides: see, for example, Younes, et al “Labelled Oligonucleotides as Radiopharmaceuticals: Pitfalls, Problems and Perspectives” Current Pharmaceutical Design, 2002, 8, 1451-14661451, the entire teachings of which are incorporated herein by reference.

Suitable fluorescent labeling agents include those known to the art, many of which are commonly commercially available, for example, fluorophores such as ALEXA 350, PACIFIC BLUE, MARINA BLUE, ACRIDINE, EDANS, COUMARIN, BODIPY 493/503, CY2, BODIPY FL-X, DANSYL, ALEXA 488, FAM, OREGON GREEN, RHODAMINE GREEN-X, TET, ALEXA 430, CAL GOLD™, BODIPY R6G-X, JOE, ALEXA 532, VIC, HEX, CAL ORANGE™, ALEXA 555, BODIPY 564/570, BODIPY TMR-X, QUASAR™ 570, ALEXA 546, TAMRA, RHODAMINE RED-X, BODIPY 581/591, CY3.5, ROX, ALEXA 568, CAL RED™, BODIPY TR-X, ALEXA 594, BODIPY 630/650-X, PULSAR™ 650, BODIPY 630/665-X, ALEXA 647 and QUASAR™ 670. Fluorescent labeling agents can include other known fluorophors, or proteins known to the art, for example, green fluorescent protein. The disclosed compounds can be coupled to the fluorescent labeling agents, administered to a subject or a sample, and the subject/sample examined by fluorescence spectroscopy or imaging to detect the labeled compound.

Quantum dots, e.g, semiconductor particles, can be employed as described in Gao, et al “In vivo cancer targeting and imaging with semiconductor quantum dots”, Nature Biotechnology, 22, (8), 2004, 969-976, the entire teachings of which are incorporated herein by reference. The disclosed compounds can be coupled to the quantum dots, administered to a subject or a sample, and the subject/sample examined by fluorescence spectroscopy or imaging to detect the labeled compound.

Numerous magnetic resonance imaging (MRI) contrast agents are known to the art, for example, positive contrast agents and negative contrast agents. The disclosed compounds can be coupled to the MRI agents, administered to a subject or a sample, and the subject/sample examined by MRI or imaging to detect the labeled compound. Positive contrast agents (typically appearing predominantly bright on MRI) can include typically small molecular weight organic compounds that chelate or contain an active element having unpaired outer shell electron spins, e.g., gadolinium, manganese, iro, or the like. Typical contrast agents include gadopentetate dimeglumine, gadoteridol, gadoterate meglumine, mangafodipir trisodium, gadodiamide, and others known to the art. Negative contrast agents (typically appearing predominantly dark on MRI) can include small particulate aggregates comprised of superparamagnetic materials, for example, particles of superparamagnetic iron oxide (SPIO). Negative contrast agents can also include compounds that lack the hydrogen atoms associated with the signal in MRI imaging, for example, perfluorocarbons (perfluorochemicals).

Also contemplated within the invention are compositions and kits comprising at least one disclosed compound. The compositions and kits may optionally contain one or more additional therapeutic agents.

A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

The terms “treat” and “treatment,” as used herein, refer to the alleviation, e.g., amelioration of one or more symptoms or effects associated with the disease, prevention, inhibition or delay of the onset of one or more symptoms or effects of the disease, and/or lessening of the severity or frequency of one or more symptoms or effects of the disease, such as the symptoms and effects described herein.

The terms “improve”, “increase” or “reduce,” as used herein, indicate values that are relative to a baseline measurement, such as a measurement in the same individual prior to initiation of the treatment described herein, or a measurement in a control individual (or multiple control individuals) in the absence of the treatment described herein. A control individual is an individual afflicted with the same disorder as the individual being treated, who is about the same age as the individual being treated (to ensure that the stages of the disease in the treated individual and the control individual are comparable).

An “effective amount” is the quantity of compound in which a beneficial clinical outcome is achieved when the compound is administered to a subject in need of treatment. The disclosed compound or additional therapeutic agent can be administered in an “effective amount” (i.e., a dosage amount that, when administered at regular intervals, is sufficient to treat the disease, such as by ameliorating symptoms associated with the disease, preventing or delaying the onset of the disease, and/or also lessening the severity or frequency of symptoms of the disease, as described above). Thus, an effective amount of the agents or compositions of the invention is a quantity which can result in a therapeutic or prophylactic benefit for the subject. The effective amount can vary, depending on such factors as the route of administration, the condition of the patient, the nature and extent of the disease's effects, and the like. Such factors are capable of determination by those skilled in the art.

As used herein, the term “effective amount” also means the total amount of each active component of the composition or method that is sufficient to show a meaningful patient benefit, i.e., treatment, healing, prevention or amelioration of the relevant medical condition, or an increase in rate of treatment, healing, prevention or amelioration of such conditions. For example, an effective amount of a compound is an amount sufficient to achieve a desired therapeutic and/or prophylactic effect, e.g., to thereby treat a cancer or symptom thereof. When applied to a combination, the term refers to combined amounts of the active ingredients that result in the therapeutic effect, whether administered in combination, serially or simultaneously.

For example, for a subject with cancer, a “beneficial clinical outcome” compared with the absence of the treatment includes a reduction in the severity of the symptoms associated with the cancer, e.g., pain, swelling, fever, rash, and the like, a reduction in the rate of cancer cell growth (e.g., reduction in tumor size, reduction in tumor vasculature, inhibition of growth of tumor or tumor neovaculature), an increase in the longevity of the subject, and the like.

The precise amount of compound administered to a subject will depend on the type and severity of the disease or condition and on the characteristics of the subject, such as general health, age, sex, body weight and tolerance to drugs. It will also depend on the degree, severity and type of inflammatory disorder. The skilled artisan will be able to determine appropriate dosages depending on these and other factors.

The disclosed compounds and additional therapeutic agents described herein can be administered to a subject by any conventional method of drug administration, for example, orally in capsules, suspensions or tablets or by parenteral administration. Parenteral administration can include, for example, intramuscular, intravenous, intraventricular, intraarterial, intrathecal, subcutaneous, or intraperitoneal administration. The disclosed compounds can also be administered orally (e.g., in capsules, suspensions, tablets or dietary), nasally (e.g., solution, suspension), transdermally, intradermally, topically (e.g., cream, ointment), inhalation (e.g., intrabronchial, intranasal, oral inhalation or intranasal drops) transmucosally or rectally. Delivery can also be by injection into the brain or body cavity of a patient or by use of a timed release or sustained release matrix delivery systems, or by onsite delivery using micelles, gels and liposomes. Nebulizing devices, powder inhalers, and aerosolized solutions may also be used to administer such preparations to the respiratory tract. Delivery can be in vivo, or ex vivo. Administration can be local or systemic as indicated. More than one route can be used concurrently, if desired. The preferred mode of administration can vary depending upon the particular disclosed compound chosen.

In specific embodiments, oral, parenteral, or system administration are preferred modes of administration for treatment of inflammatory disorders.

The compounds can be administered alone as a monotherapy, or in conjunction with one or more additional therapeutic agents. The term “in conjunction with,” indicates that the compound is administered at about the same time as the agent. The compound can be administered to the animal as part of a pharmaceutical composition comprising the compound and a pharmaceutically acceptable carrier or excipient and, optionally, one or more additional therapeutic agents. The compound and compound can be components of separate pharmaceutical compositions which can be mixed together prior to administration or administered separately. The compound can, for example, be administered in a composition containing the additional therapeutic agent, and thereby, administered contemporaneously with the agent. Alternatively, the compound can be administered contemporaneously, without mixing (e.g., by delivery of the compound on the intravenous line by which the compound is also administered, or vice versa). In another embodiment, the compound can be administered separately (e.g., not admixed), but within a short time frame (e.g., within 24 hours) of administration of the compound.

The methods of the present invention contemplate single as well as multiple administrations, given either simultaneously or over an extended period of time. The compound (or composition containing the compound) can be administered at regular intervals, depending on the nature and extent of the inflammatory disorder's effects, and on an ongoing basis. Administration at a “regular interval,” as used herein, indicates that the therapeutically effective amount is administered periodically (as distinguished from a one-time dose). In one embodiment, the compound is administered periodically, e.g., at a regular interval (e.g., bimonthly, monthly, biweekly, weekly, twice weekly, daily, twice a day or three times or more often a day).

The administration interval for a single individual can be fixed, or can be varied over time, depending on the needs of the individual. For example, in times of physical illness or stress, or if disease symptoms worsen, the interval between doses can be decreased. Depending upon the half-life of the agent in the subject, the agent can be administered between, for example, once a day or once a week.

For example, the administration of the disclosed compound and/or the additional therapeutic agent can take place at least once on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least once on week 1, 2, 3, 4, 5, 6, 7, 8, 9, 10, 11, 12, 13, 14, 15, 16, 17, 18, 19 or 20, or any combination thereof, using single or divided doses of every 60, 48, 36, 24, 12, 8, 6, 4, or 2 hours, or any combination thereof. Administration can take place at any time of day, for example, in the morning, the afternoon or evening. For instance, the administration can take place in the morning, e.g, between 6:00 a.m. and 12:00 noon; in the afternoon, e.g., after noon and before 6:00 p.m.; or in the evening, e.g., between 6:01 p.m. and midnight. The compound can be administered before, during or after the onset of the inflammatory disorder.

The disclosed compound and/or additional therapeutic agent can be administered in a dosage of, for example, 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day. Dosage forms (composition) suitable for internal administration generally contain from about 0.1 milligram to about 500 milligrams of active ingredient per unit. In these pharmaceutical compositions the active ingredient will ordinarily be present in an amount of about 0.5-95% by weight based on the total weight of the composition.

The amount of disclosed compound and/or additional therapeutic agent administered to the subject can depend on the characteristics of the subject, such as general health, age, sex, body weight and tolerance to drugs as well as the degree, severity and type of rejection. The skilled artisan will be able to determine appropriate dosages depending on these and other factors using standard clinical techniques.

In addition, in vitro or in vivo assays can be employed to identify desired dosage ranges. The dose to be employed can also depend on the route of administration, the seriousness of the disease, and the subject's circumstances. Effective doses may be extrapolated from dose-response curves derived from in vitro or animal model test systems. The amount of the compound can also depend on the disease state or condition being treated along with the clinical factors and the route of administration of the compound.

For treating humans or animals, the amount of disclosed compound and/or additional therapeutic agent administered (in milligrams of compound per kilograms of subject body weight) is generally from about 0.1 mg/kg to about 100 mg/kg, typically from about 1 mg/kg to about 50 mg/kg, or more typically from about 1 mg/kg to about 25 mg/kg. In a preferred embodiment, the effective amount of agent or compound is about 1-10 mg/kg. In another preferred embodiment, the effective amount of agent or compound is about 1-5 mg/kg. The effective amount for a subject can be varied (e.g., increased or decreased) over time, depending on the needs of the subject.

The term “unit dose” refers to a physically discrete unit suitable as unitary dosage for the subject, each unit containing a predetermined quantity of active material that can produce the desired therapeutic effect in association with the required diluent; e.g., carrier or vehicle. In addition to the ingredients particularly mentioned above, the formulations of the present invention may include other agents conventional in the art having regard to the type of formulation in question.

The disclosed compound and/or additional therapeutic agent described herein can be administered to the subject in conjunction with an acceptable pharmaceutical carrier or diluent as part of a pharmaceutical composition for therapy. Formulation of the compound to be administered will vary according to the route of administration selected (e.g., solution, emulsion, capsule, and the like). Suitable pharmaceutically acceptable carriers may contain inert ingredients which do not unduly inhibit the biological activity of the compounds. The pharmaceutically acceptable carriers should be biocompatible, e.g., non-toxic, non-inflammatory, non-immunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

The preparation of a pharmacological composition that contains active ingredients dissolved or dispersed therein is well understood in the art. Typically such compositions are prepared as injectables either as liquid solutions or suspensions, however, solid forms suitable for solution, or suspensions, in liquid prior to use can also be prepared. Formulation will vary according to the route of administration selected (e.g., solution, emulsion, capsule).

A pharmaceutically acceptable carrier for pharmaceutical composition can also include delivery systems known to the art for entraining or encapsulating drugs such as anticancer drugs. In some embodiments, the disclosed compounds can be employed with such delivery systems including, for example, liposomes, nanoparticles, nanospheres, nanodiscs, dendrimers, and the like. See, for example Farokhzad, O. C., Jon, S., Khademhosseini, A., Tran, T. N., Lavan, D. A., and Langer, R. (2004). “Nanoparticle-aptamer bioconjugates: a new approach for targeting prostate cancer cells.” Cancer Res., 64, 7668-72; Dass, C. R. (2002). “Vehicles for oligonucleotide delivery to tumours.” J. Pharm. Pharmacol., 54, 3-27; Lysik, M. A., and Wu-Pong, S. (2003). “Innovations in oligonucleotide drug delivery.” J. Pharm. Sci., 92, 1559-73; Shoji, Y., and Nakashima, H. (2004). “Current status of delivery systems to improve target efficacy of oligonucleotides.” Curr. Pharm. Des., 10, 785-96; Allen, T. M., and Cullis, P. R. (2004). “Drug delivery systems: entering the mainstream.” Science, 303, 1818-22. The entire teachings of each reference cited in this paragraph are incorporated herein by reference.

In one embodiment, the method comprises topical administration. In such cases, the compounds may be formulated as a solution, gel, lotion, cream or ointment in a pharmaceutically acceptable form. Actual methods for preparing these, and other, topical pharmaceutical compositions are known or apparent to those skilled in the art and are described in detail in, for example, Remington's Pharmaceutical Sciences, 16^{th} and 18^{th} eds., Mack Publishing Company, Easton, Pa., 1980-1990).

As used herein, the term “pharmaceutically acceptable”, means that the materials (e.g., compositions, carriers, diluents, reagents, salts, and the like) can be administered to or upon a subject with a minimum of undesirable physiological effects such as nausea, dizziness or gastric upset.

Also included in the present invention are pharmaceutically acceptable salts of the PMSA agents. “Pharmaceutically acceptable” means that the cation is suitable for administration to a subject.

The disclosed compounds can have one or more sufficiently acidic protons that can react with a suitable organic or inorganic base to form a base addition salt. For example, when a compound has a hydrogen atom bonded to an oxygen, nitrogen, or sulfur atom, it is contemplated that the compound also includes salts thereof wherein the hydrogen atom has been reacted with a suitable organic or inorganic base to form a base addition salt. Base addition salts include those derived from inorganic bases, such as ammonium or alkali or alkaline earth metal hydroxides, carbonates, bicarbonates, and the like, and organic bases such as alkoxides, alkyl amides, alkyl and aryl amines, and the like. Such bases useful in preparing the salts of this invention thus include sodium hydroxide, potassium hydroxide, ammonium hydroxide, potassium carbonate, the like. Pharmaceutically acceptable salts can be those formed by reaction with one equivalent of a suitable base to form a monovalent salt (e.g., the compound has single negative charge that is balanced by a pharmaceutically acceptable counter cation, e.g., a monovalent cation) or with two equivalents of a suitable base to form a divalent salt (e.g., the compound has a two-electron negative charge that can be balanced by two pharmaceutically acceptable counter cations, e.g., two pharmaceutically acceptable monovalent cations or a single pharmaceutically acceptable divalent cation). Examples include Li⁺, Na⁺, K⁺, Mg²⁺, Ca²⁺ and NR₄⁺, wherein each R is independently hydrogen, an optionally substituted aliphatic group (e.g., a hydroxyalkyl group, aminoalkyl group or ammoniumalkyl group) or optionally substituted aryl group, or two R groups, taken together, form an optionally substituted non-aromatic heterocyclic ring optionally fused to an aromatic ring. Generally, the pharmaceutically acceptable cation can be Li⁺, Na⁺, K⁺, NH₃(C₂H₅OH)⁺ or N(CH₃)₃(C₂H₅OH)⁺.

The disclosed compounds with a sufficiently basic group, such as an amine, can react with an organic or inorganic acid to form an acid addition salt. Acids commonly employed to form acid addition salts from compounds with basic groups can be inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts which can be formed include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

It will also be understood that certain disclosed compounds may be obtained as different stereoisomers (e.g., diastereomers and enantiomers) and that the invention includes all isomeric forms and racemic mixtures of the disclosed compounds and methods of treating a subject with both pure isomers and mixtures thereof, including racemic mixtures. Stereoisomers can be separated and isolated using any suitable method, such as chromatography.

### Other Pmsa Ligands

### Introduction (A)

In various embodiments, the PSMA ligands represented by variable A or L-A in Structural Formulas A1, A3, A11, A12, and A14 are those ligands known in the art to target PSMA and other GCP2s such as NAALADase. Thus, as used herein, “PSMA ligands” also includes those compounds or groups known as NAALADase ligands. In typical embodiments, these PSMA ligands are those generically or specifically described in PSMA Ligand Sections B-G, or the documents incorporated by reference therein.

### PMSA Ligands (B.1)

In various embodiments, the PSMA ligands represented by variable A in Structural Formulas A1, A2, A3, A11, A12, and A14 can be those described in U.S. Published Patent Application No. US2004/0002478, or the patents and applications to which it claims priority, U.S. Pat. Nos. 6,528,499; 6,479,470 and U.S. Provisional Patent Application Nos. 60/131,627, filed Apr. 28, 1999; 60/166,915, filed Nov. 22, 1999; and 60/188,031, filed Mar. 9, 2000. The entire teachings of each of these documents are incorporated herein by reference. The variables and terms in this section can be as described herein, more typically as described in this section, or in preferred embodiments can be as described in the documents incorporated by reference in this paragraph.

In various embodiments, the PSMA ligands represented by variable A in Structural Formulas A1, A2, A3, A11, A12, and A14 can be represented by the following Structural Formula:

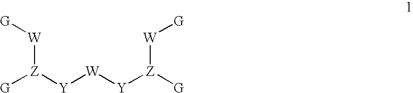

X is selected from the group consisting of —C(O)—, —C(S)—, —P(O)(OR)—, —S(O)₂—, —C(R)(OR)—, and —C(R)(SR)—;

Y is selected, independently for each occurrence, from the group consisting of (CR₂)ₙ, (NR)ₙ, and a bond;

Z is selected, independently for each occurrence, from the group consisting of C(R), C(NR₂), and C(NHacyl);

W is selected, independently for each occurrence, from the group consisting of (CR₂)ₘ, (NR)ₘ, and a bond;

G is selected, independently for each occurrence, from the group consisting of H, —COOH, —SO₃H, —P(O)(OH)₂, —SR, and 2-R-tetrazol-5-yl;

R is selected, independently for each occurrence, from the group consisting of H, alkyl, heteroalkyl, aryl, heteroaryl, and aralkyl; and also including a negative charge for instances of R bonded to a heteroatom;

m and n are integers selected, independently for each occurrence, from the range 0 to 3 inclusive; and

the stereochemical configuration at any stereocenter of a compound represented by 1 is R, S, or a mixture of these configurations.

In various embodiments, the PSMA ligands of this section are represented by structure 1 and the attendant definitions, wherein X is —C(O)—.

In various embodiments, the PSMA ligands of this section are represented by structure 1 and the attendant definitions, wherein Y is independently for each occurrence (NR)ₙ.

In various embodiments, the PSMA ligands of this section are represented by structure 1 and the attendant definitions, wherein Z is independently for each occurrence C(R).

In various embodiments, the PSMA ligands of this section are represented by structure 1 and the attendant definitions, wherein W is independently for each occurrence (CR₂)ₘ.

In various embodiments, the PSMA ligands of this section are represented by structure 1 and the attendant definitions, wherein G is selected, independently for each occurrence, from the group consisting of H, —COOH, —SR, and 2-R-tetrazol-5-yl.

In various embodiments, the PSMA ligands of this section are represented by structure 1 and the attendant definitions, wherein m and n are integers selected, independently for each occurrence, from 1 and 2.

In various embodiments, the PSMA ligands of this section are represented by structure 1 and the attendant definitions, wherein X is —C(O)—; and Y is independently for each occurrence (NR)ₙ

In various embodiments, the PSMA ligands of this section are represented by structure 1 and the attendant definitions, wherein X is —C(O)—; and Z is independently for each occurrence C(R).

In various embodiments, the PSMA ligands of this section are represented by structure 1 and the attendant definitions, wherein X is —C(O)—; and W is independently for each occurrence (CR₂)ₘ.

In various embodiments, the PSMA ligands of this section are represented by structure I and the attendant definitions, wherein X is —C(O)—; and G is selected, independently for each occurrence, from the group consisting of H, —COOH, —SR, and 2-R-tetrazol-5-yl.

In various embodiments, the PSMA ligands of this section are represented by structure 1 and the attendant definitions, wherein X is —C(O)—; Y is independently for each occurrence (NR)ₙ; and Z is independently for each occurrence C(R).

In various embodiments, the PSMA ligands of this section are represented by structure 1 and the attendant definitions, wherein X is —C(O)—; Y is independently for each occurrence (NR)ₙ; and W is independently for each occurrence (CR₂)ₘ.

In various embodiments, the PSMA ligands of this section are represented by structure 1 and the attendant definitions, wherein X is —C(O)—; Y is independently for each occurrence (NR)ₙ; and G is selected, independently for each occurrence, from the group consisting of H, —COOH, —SR, and 2-R-tetrazol-5-yl.

In various embodiments, the PSMA ligands of this section are represented by structure 1 and the attendant definitions, wherein X is —C(O)—; Y is independently for each occurrence (NR)ₙ; Z is independently for each occurrence C(R); and W is independently for each occurrence (CR₂)ₘ.

In various embodiments, the PSMA ligands of this section are represented by structure 1 and the attendant definitions, wherein X is —C(O)—; Y is independently for each occurrence (NR)ₙ; W is independently for each occurrence (CR₂)ₘ; and G is selected, independently for each occurrence, from the group consisting of H, —COOH, —SR, and 2-R-tetrazol-5-yl.

In various embodiments, the PSMA ligands of this section are represented by structure 1 and the attendant definitions, wherein X is —C(O)—; Y is independently for each occurrence (NR)ₙ; Z is independently for each occurrence C(R); W is independently for each occurrence (CR₂)ₘ; and G is selected, independently for each occurrence, from the group consisting of H, —COOH, —SR, and 2-R-tetrazol-5-yl.

In various embodiments, the PSMA ligands represented by variable A in Structural Formulas A1, A2, A3, A11, A12, and A14 can be represented by the following Structural Formula:

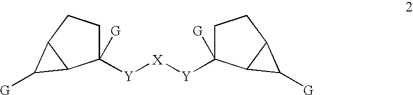

X is selected from the group consisting of —C(O)—, —C(S)—, —P(O)(OR)—, —S(O)₂—, —C(R)(OR)—, and —C(R)(SR)—;

Y is selected, independently for each occurrence, from the group consisting of (CR₂)ₙ, (NR)ₙ, and a bond;

G is selected, independently for each occurrence, from the group consisting of H, —COOH, —SO₃H, —P(O)(OH)₂, and 2-R-tetrazol-5-yl;

R is selected, independently for each occurrence, from the group consisting of H, alkyl, heteroalkyl, aryl, heteroaryl, and aralkyl; and also including a negative charge for instances of R bonded to a heteroatom;

n is an integer selected, independently for each occurrence, from the range 0 to 3 inclusive; and

the stereochemical configuration at any stereocenter of a compound represented by 2 is R, S, or a mixture of these configurations.

In various embodiments, the PSMA ligands of this section are represented by structure 2 and the attendant definitions, wherein X is —C(O)—.

In various embodiments, the PSMA ligands of this section are represented by structure 2 and the attendant definitions, wherein Y is independently for each occurrence (NR)ₙ.

In various embodiments, the PSMA ligands of this section are represented by structure 2 and the attendant definitions, wherein G is selected, independently for each occurrence, from the group consisting of —COOH, —SO₃H, —P(O)(OH)₂, and 2-R-tetrazol-5-yl.

In various embodiments, the PSMA ligands of this section are represented by structure 2 and the attendant definitions, wherein G is selected, independently for each occurrence, from the group consisting of —COOH, and 2-R-tetrazol-5-yl.

In various embodiments, the PSMA ligands of this section are represented by structure 2 and the attendant definitions, wherein X is —C(O)—; and Y is independently for each occurrence (NR)ₙ.

In various embodiments, the PSMA ligands of this section are represented by structure 2 and the attendant definitions, wherein X is —C(O)—; Y is independently for each occurrence (NR)ₙ; and G is selected, independently for each occurrence, from the group consisting of —COOH, —SO₃H, —P(O)(OH)₂, and 2-R-tetrazol-5-yl.

In various embodiments, the PSMA ligands of this section are represented by structure 2 and the attendant definitions, wherein X is —C(O)—; Y is independently for each occurrence (NR)ₙ; and G is selected, independently for each occurrence, from the group consisting of —COOH, and 2-R-tetrazol-5-yl.

In various embodiments, a compound of the present invention is represented by structure 1 or 2 and the attendant definitions, wherein the compound is a single stereoisomer.

### PMSA Ligands (B.2)

In various embodiments, the PSMA ligands represented by variable A in Structural Formulas A1, A2, A3, A11, A12, and A14 can be those described in U.S. Pat. No. 6,313,159. The entire teachings of this document are incorporated herein by reference. The variables and terms in this section can be as described herein, more typically as described in this section, or in preferred embodiments can be as described in the documents incorporated by reference in this paragraph.

In various embodiments, the PSMA ligands represented by variable A in Structural Formulas A1, A2, A3, A11, A12, and A14 can be represented by the following Structural Formula:

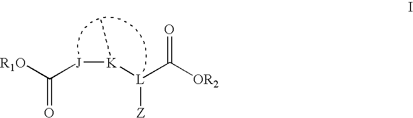

wherein:

either J and K are taken together with one or more additional atoms independently selected from the group consisting of C, O, S, and N in chemically reasonable substitution patterns to form a 3-7 membered saturated or unsaturated heterocyclic or carbocyclic ring, and L is —CH,

or J, K, and L are taken together with one or more additional atoms independently selected from the group consisting of C, O, S, and N in chemically reasonable substitution patterns to form a 4-8 membered saturated or unsaturated, mono-, bi-, or tricyclic, hetero- or carbocyclic ring structure;

Z is a metal chelating group;

R₁ and R₂ are independently hydrogen, C₁-C₉ alkyl, C₂-C₈ alkenyl, C3-C8 cycloalkyl, C₅-C₇ cycloalkenyl, or Ar, wherein each said alkyl, alkenyl, cycloalkyl, cycloalkenyl, or Ar is independently unsubstituted or substituted with one or more substituent(s); and

Ar is a carbocyclic or heterocyclic moiety which is unsubstituted or substituted with one or more substituent(s).

In a preferred embodiment of formula I, R₁ and R₂ are each hydrogen. In various embodiments, the PSMA ligands represented by variable A in Structural Formulas A1, A2, A3, A11, A12, and A14 can be represented by the following Structural Formula:

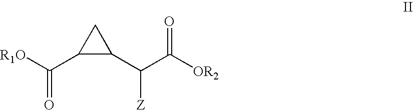

wherein:

Z is a metal chelating group;

R₁ and R₂ are independently hydrogen, C₁-C₉ alkyl, C₂-C₈ alkenyl, C₃-C₈ cycloalkyl, C₅-C₇ cycloalkenyl, or Ar, wherein each said alkyl, alkenyl, cycloalkyl, cycloalkenyl, or Ar is independently unsubstituted or substituted with one or more substituent(s); and

Ar is a carbocyclic or heterocyclic moiety which is unsubstituted or substituted with one or more substituent(s).

In a preferred embodiment of formula II, R₁ and R₂ are each hydrogen.

In various embodiments, the PSMA ligands represented by variable A in Structural Formulas A1, A2, A3, A11, A12, and A14 can be represented by the following Structural Formula:

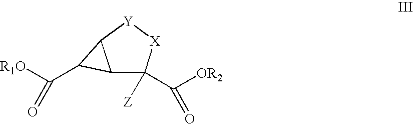

wherein:

X and Y are independently selected from the group consisting of CH₂, O, NH, or S;

Z is a metal chelating group;

R₁ and R₂ are independently hydrogen, C₁-C₉ alkyl, C₂-C₉ alkenyl, C₃-C₈ cycloalkyl, C₅-C₇ cycloalkenyl, or Ar, wherein each said alkyl, alkenyl, cycloalkyl, cycloalkenyl, or Ar is independently unsubstituted or substituted with one or more substituent(s); and

Ar is a carbocyclic or heterocyclic moiety which is unsubstituted or substituted with one or more substituent(s).

In a preferred embodiment of formula III, R₁ and R₂ are each hydrogen.

In various embodiments, the PSMA ligands represented by variable A in Structural Formulas A1, A2, A3, A11, A12, and A14 can be selected from the group consisting of:
4-(phosphonomethyl)-2,4-pyrrolidine dicarboxylic acid (1);
4-[[hydroxy(phenyl)phosphinyl]methyl]-2,4-pyrrolidinedicarboxylic acid (2);
4-[[hydroxy(phenylmethyl)phosphinyl]methyl]-2,4-pyrrolidinedicarboxylic acid (3);
4-[[hydroxy(phenylethyl)phosphinyl]methyl]-2,4-pyrrolidinedicarboxylic acid (4);
4-(sulfanylmethyl)-2,4-pyrrolidine dicarboxylic acid (5); and

pharmaceutically acceptable equivalents thereof.

In various embodiments, the PSMA ligands represented by variable A in Structural Formulas A1, A2, A3, A11, A12, and A14 can be selected from the group consisting of:
2-carboxy-α-(phosphonomethyl)-cyclopropaneacetic acid (16);
2-carboxy-α-[[hydroxypropylphosphinyl]methyl]cyclopropaneacetic acid (17);
2-carboxy-α-[[butylhydroxyphosphinyl]methyl]cyclopropaneacetic acid (18);
2-carboxy-α-[[hydroxyphenylphosphinyl]methyl]cyclopropaneacetic acid (19);
2-carboxy-α-[[hydroxy(phenylmethyl)phosphinyl]methyl]-cyclopropaneacetic acid (20);
2-carboxy-α-[[hydroxy(2-phenylethyl)phosphinyl]methyl]-cyclopropaneac etic acid (21);
2-carboxy-α-(mercaptoethyl)-cyclopropaneacetic acid (23);
2-carboxy-α-(mercaptopropyl)-cyclopropaneacetic acid (24);
2-carboxy-α-[2-(hydroxyamino)-2-oxoethyl]cyclopropaneacetic acid (25);
2-carboxy-α-[3-(hydroxyamino)-3-oxopropyl]cyclopropaneacetic acid (26);
2-carboxy-α-[(carboxymethyl)amino]cyclopropaneacetic acid (27);
2-carboxy-α-[[(carboxymethyl)amino]methyl]cyclopropaneacetic acid (28).

In various embodiments, the PSMA ligands represented by variable A in Structural Formulas A1, A2, A3, Al1, A12, and A14 can be selected from the group consisting of:
2-(phosphonomethyl)-bicyclo[3.1.0]hexane-2,6-dicarboxylic acid (29);
2-[[hydroxy(ethylphosphinyl)methyl)-3-oxabicyclo03.1.0]hexane-2,6-dicarboxylic acid (30);
2-[[hydroxy(phenylmethyl)phosphinyl]methyl]; and
bicyclo[3.1.0]hexane-2,6-dicarboxylic acid (31).
Preferred compounds of formula III can be selected from the group consisting of:
2-oxa-4-(ethylhydroxyphosphoryl)aminobicyclo[3.1.0]hexane-4,6-dicarboxylate;
2-thia-4-(ethylhydroxyphosphoryl)aminobicyclo[3.1.0]hexane-4,6-dicarboxylate;
2-oxa-4-(hydroxyphosphoryl)bicyclo[3.1.0]hexane-4,6-dicarboxylate;
2-thia-4-(hydroxyphosphoryl)bicyclo[3.1.0]hexane-4,6-dicarboxylate;
2-oxa-4-(methylsulfanyl)bicyclo[3.1.0]hexane-4,6-dicarboxylate;
2-thia-4-(methylsulfanyl)bicyclo[3.1.0]hexane-4,6-dicarboxylate; and
4-[[hydroxy(phenylmethyl)phosphinyl]methyl]-2-oxabicyclo[3.1.0]hexane-4,6-dicarboxylic acid.

Tables I-IV(PSMA Ligands Section B.2)

TABLE I

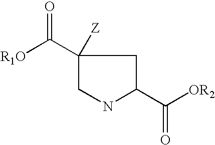

| Compound | R₁ | R₂ | Z |
|---|---|---|---|
| 1 | H | H | —CH₂P(O)(OH)₂ |
| 2 | H | H | 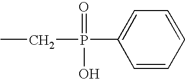 |
| 3 | H | H | 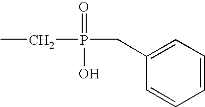 |
| 4 | H | H | 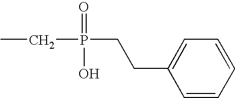 |
| 5 | H | H | —CH₂SH |
| 6 | H | H | 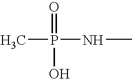 |
| 7 | H | H | 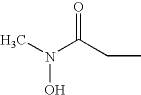 |
| 8 | H | H | 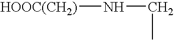 |
| 9 | phenyl | H | —CH₂P(O)(OH)₂ |
| 10 | —CH₃ | —CH₃ | —CH₂P(O)(OH)CH₂CH₃ |
| 11 | H | —CH₃ | 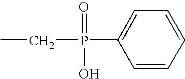 |
| 12 | 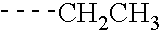 | phenyl | —CH₂CH₂SH |
| 13 | cyclohexyl | —CH₂SH | 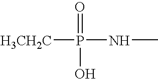 |
| 14 | trifluoromethyl | —NH₂ | 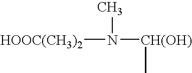 |
| 15 | pyridyl | benzyl | 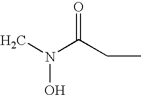 |

TABLE II

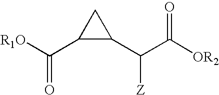

| Compound | R₁ | R₂ | Z |
|---|---|---|---|
| 16 | H | H | —CH₂P(O)(OH)₂ |
| 17 | H | H | —CH₂P(O)(OH)CH₂CH₂CH₃ |
| 18 | H | H | —CH₂P(O)(OH)CH₂CH₂CH₂CH₃ |
| 19 | H | H | 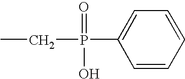 |
| 20 | H | H | 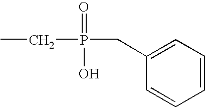 |
| 21 | H | H | 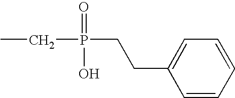 |
| 22 | H | H | 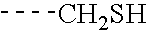 |
| 23 | H | H | 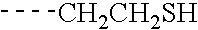 |
| 24 | H | H | 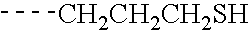 |
| 25 | H | H | 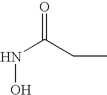 |
| 26 | H | H | 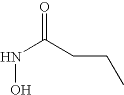 |
| 27 | H | H | —NHCH₂COOH |
| 28 | H | H | —CH₂NHCH₂COOH |

TABLE III

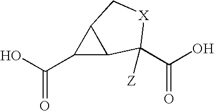

| Compound | X | Z |
|---|---|---|
| 29 | CH₂ | —CH₂P(O)(OH)₂ |
| 30 | O | —CH₂P(O)(OH)CH₂CH₃ |
| 31 | CH₂ | 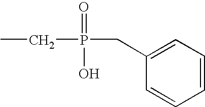 |

TABLE IV

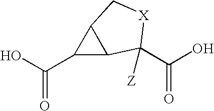

| Compound | Y | Z |
|---|---|---|
| 32 | O | 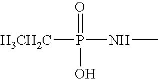 |
| 33 | S | 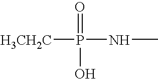 |
| 34 | O | —CH₂P(O)(OH)₂ |
| 35 | S | —CH₂P(O)(OH)₂ |
| 36 | O | —CH₂SH |
| 37 | S | —CH₂SH |
| 38 | O | —CH₂P(O)(OH)CH₂CH₃ |
| 39 | S | —CH₂P(O)(OH)CH₂CH₃ |
| 40 | O | 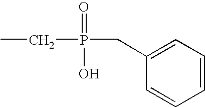 |
| 41 | S | 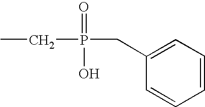 |

### PMSA Ligands (C.1)

In some embodiments, the PSMA ligand can be selected from those described in U.S. Published Patent Application No. US2003/0083374, or the patents and applications to which it claims priority, U.S. Pat. Nos. 6,395,718 and 6,265,609, and U.S. patent application Ser. No. 09/346,711, filed Jul. 2, 1999 and 09/110,186, filed Jul. 6, 1998 (now abandoned). The entire teachings of each of these documents are incorporated herein by reference. The structural variables, Structural Formulas, and terms in this section can be as described herein, more typically as described in this section, or in preferred embodiments can be as described in the documents incorporated by reference in this paragraph.

In various embodiments, the PSMA ligands represented by variable A in Structural Formulas A1, A2, A3, A11, A12, and A14 can be represented by the following Structural Formula:

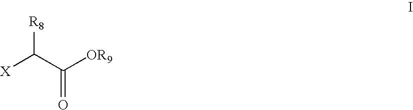

wherein:

X is a moiety of formula II, III or IV

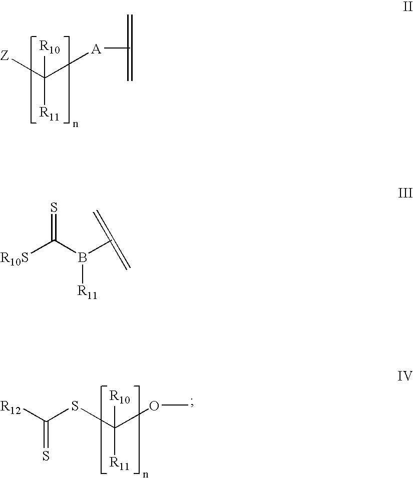

m and n are independently 0, 1, 2, 3 or 4;

Z is SR₁3, SO₃R₁3, SO₂R₁3, SOR₁3, SO(NR₁3)R₁4 or S(NR₁3)R₁4)₂R₁5;

B is N or CR₁6;

A is O, S, CR₁7R₁8 or (CR₁7R₁8)ₘS;

R₉ and R₁3 are hydrogen;

R₈, R₁₀, R₁₁, R₁₂, R₁4, R₁5, R₁6, R₁7 and R₁8 are independently hydrogen, C₁-C₉ straight or branched chain alkyl, C₂-C₉ straight or branched chain alkenyl, C₃-C₈ cycloalkyl, C₅-C₇ cycloalkenyl, Ar₁, hydroxy, carboxy, carbonyl, amino, amido, cyano, isocyano, nitro, sulfonyl, sulfoxy, thio, thiocarbonyl, thiocyano, formanilido, thioformamido, sulfhydryl, halo, haloalkyl, trifluoromethyl or oxy, wherein said alkyl, alkenyl, cycloalkyl and cycloalkenyl are independently unsubstituted or substituted with one or more substituent(s); and

Ar₁ is a carbocyclic or heterocyclic moiety, which is unsubstituted or substituted with one or more substituent(s);

provided that when X is a moiety of formula II and A is O, then n is 2, 3 or 4; when X is a moiety of formula II and A is S, then n is 2, 3 or 4; and when X is a moiety of formula II and A is (CR₁7R₁8)ₘS, then n is 0, 2, 3 or 4.

In various embodiments, the PSMA ligands represented by variable A in Structural Formulas A1, A2, A3, A11, A12, and A14 can be selected from the group consisting of:
2-(2-sulfanylethyl)pentanedioic acid;
3-(2-sulfanylethyl)-1,3,5-pentanetricarboxylic acid;
2-(2-sulfanylpropyl)pentanedioic acid;
2-(2-sulfanylbutyl)pentanedioic acid;
2-(2-sulfanyl-2-phenylethyl)pentanedioic acid;
2-(2-sulfanylhexyl)pentanedioic acid;
2-(2-sulfanyl-1-methylethyl)pentanedioic acid;
2-[1-(sulfanylmethyl)propyl]pentanedioic acid;
2-(3-sulfanylpentyl)pentanedioic acid;
2-(3-sulfanylpropyl)pentanedioic acid;
2-(3-sulfanyl-2-methylpropyl)pentanedioic acid;
2-(3-sulfanyl-2-phenylpropyl)pentanedioic acid;
2-(3-sulfanylbutyl)pentanedioic acid;
2-[3-sulfanyl-2-(phenylmethyl)propyl]pentanedioic acid;
2-[2-(sulfanylmethyl)butyl]pentanedioic acid;
2-[2-(sulfanylmethyl)pentyl]pentanedioic acid; and
2-(3-sulfanyl-4-methylpentyl)pentanedioic acid.

Representative compounds of formula I wherein X is a moiety of formula III, R₈ is —(CH₂)₂COOH, R₉ is hydrogen, and B is CR₁6, include without limitation: 2-(dithiocarboxymethyl)pentanedioic acid and 2-(1-dithiocarboxyethyl)pentanedioic acid.

Representative compounds of formula I wherein X is a moiety of formula III, R₈ is —(CH₂)₂COOH, R₉ is hydrogen, and B is N, include without limitation:
2-dithiocarboxyaminopentanedioic acid;
2-[(N-methyldithiocarboxy)amino]pentanedioic acid; and

pharmaceutically acceptable equivalents.

Representative compounds of formula I wherein X is a moiety of formula IV include without limitation:
2-benzyl-4-sulfanylbutanoic acid;
2-benzyl-4-sulfanylpentanoic acid;
2-(3-pyridylmethyl)-4-sulfanylpentanoic acid;
2-(3-pyridylmethyl)-4-sulfanylhexanoic acid;
2-benzyl-3-sulfanylpropanoic acid;
2-benzyl-3-sulfanylpentanoic acid; and
2-(4-pyridylmethyl)-3-sulfanylpentanoic acid.

### PMSA Ligands (C.2)

In various embodiments, the PSMA ligands represented by variable A in Structural Formulas A1, A2, A3, A11, A12, and A14 can be those described in U.S. Pat. No. 6,348,464. The entire teachings of each of this documents are incorporated herein by reference. The variables and terms in this section can be as described herein, more typically as described in this section, or in preferred embodiments can be as described in the documents incorporated by reference in this paragraph.

In various embodiments, the PSMA ligands represented by variable A in Structural Formulas A1, A2, A3, A11, A12, and A14 can be represented by the following Structural Formula:

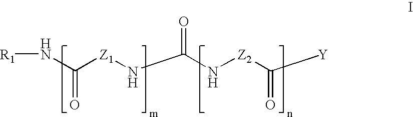

wherein:

m and n are independently 0, 1, 2 or 3;

Y is —R₂ or —NHR₂;

R₁ and R₂ are independently an aryl or heteroaryl group substituted with one or more substituent(s);

Z₁ and Z₂ are independently a moiety of formula II, III or IV

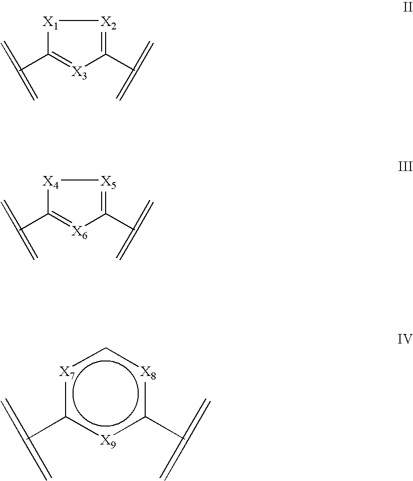

X₁ and X₅ are independently CHR₃ or NR₃;

X₂, X₃, X₄, X₆, X₇, X₈ and X₉ are independently CR₃ or N; and

R₃ is H or CH₃.

In various embodiments, the PSMA ligands represented by variable A in Structural Formulas A1, A2, A3, A11, A12, and A14 can be represented by the following Structural Formula:

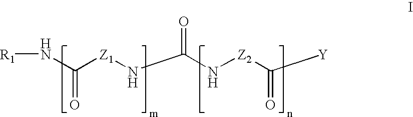

wherein:

m and n are independently 0, 1, 2 or 3;

Y is —R₂ or —NHR₂;

R₁ and R₂ are independently an aryl or heteroaryl group substituted with one or more substituent(s);

Z₁ and Z₂ are independently a moiety of formula II, III or

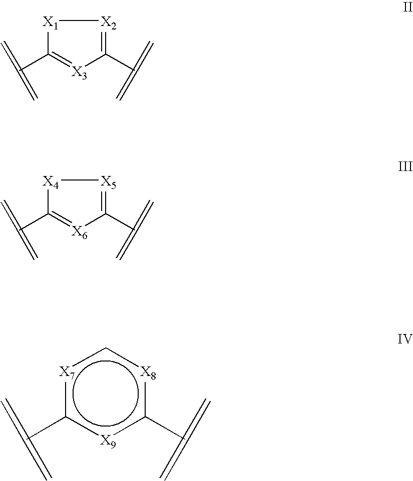

X₁ and X₅ are independently CHR₃ or NR₃;

X₂, X₃, X₄, X₆, X₇, X₈ and X₉ are independently CR₃ or N; and

R₃ is H or CH₃;

provided that when m and n are independently 1, 2 or 3, Y is —NHR₂, R₂ is naphthyl substituted with 1, 2 or 3 sulfonic acid(s), Z₁ and Z₂ are each a moiety of formula II, X₁ and X₂ are each CH, X₃ is NR₃, and R₃ is CH₃, then the glutamate abnormality is not a demyelinating disease; and when the compound of formula I is suramin, then the glutamate abnormality is not a CNS neurodegenerative disorder or a demyelinating disease.

In various embodiments, the PSMA ligands represented by variable A in Structural Formulas A1, A2, A3, A11, A12, and A14 can be selected from the group consisting of:

suramin;
2,2′-(carbonyl-bis(imino-N-methyl-4,2-pyrrolecarbonylimino(N-methyl-4,2-pyrrole)carbonylimino))bis(1,5-naphthalenedisulfonic acid);
8,8′-[carbonylbis(imino-3,1-phenylene)]bis-(1,3,5-naphthalenetrisulfonic acid);
5-[[[1-methyl-4-[[[[1-methyl-6-[[(4,5,7-trisulfo-1-naphthalenyl)amino]carbonyl]-1H-pyrrol-3-yl]amino]carbonyl]amino]-1H-pyrrol-2-yl]carbonyl]amino]-1,3,8-naphthalenetrisulfonic acid;
4-[[[1-methyl-4-[[[[1-methyl-5-[[(4-sulfo-1-naphthalenyl)amino]carbonyl]-1H-pyrrol-3-yl]amino]carbonyl]amino]-1H-pyrrol-2-yl]carbonyl]amino]-1-naphthalenesulfonic acid;
7,7′-(carbonyl-bis(imino-N-methyl-4,2-pyrrolecarbonylimino(N-methyl-4,2-pyrrole)carbonylimino))bis(3,5-naphthalenedisulfonic acid);
7,7′-(carbonyl-bis(imino-N-methyl-4,2-pyrrolecarbonylimino(N-methyl-4,2-pyrrole)carbonylimino))bis(3,6-naphthalenedisulfonic acid);
7,7′-(carbonyl-bis(imino-N-methyl-4,2-pyrrolecarbonylimino(N-methyl-4,2-pyrrole)carbonylimino))bis(1,3,5-naphthalenetrisulfonic acid);
8,8′-(carbonyl-bis(imino-N-methyl-4,2-pyrrolecarbonylimino(N-methyl-4,2-pyrrole)carbonylimino))bis(1,3,6-naphthalenetrisulfonic acid);
7,7′-(carbonyl-bis(imino-N-methyl-4,2-pyrrolecarbonylimino(N-methyl-4,2-pyrrole)carbonylimino))bis(1,3-naphthalenedisulfonic acid);
7,7′-(carbonyl-bis(imino-N-methyl-4,2-pyrrolecarbonylimino(N-methyl-4,2-pyrrole)carbonylimino))bis(2,4-naphthalenedisulfonic acid);
8,8′-(carbonyl-bis(imino-N-methyl-4,2-pyrrolecarbonylimino(N-methyl-4,2-pyrrole)carbonylimino))bis(2,4-naphthalenedisulfonic acid);
8,8′-(carbonyl-bis(imino-N-methyl-4,2-pyrrolecarbonylimino(N-methyl-4,2-pyrrole)carbonylimino))bis(1,3,5-naphthalenetrisulfonic acid);
8,8′-(carbonyl-bis(imino-N-methyl-4,2-pyrrolecarbonylimino(N-methyl-4,2-pyrrole)carbonylimino))bis(5-naphthalenesulfonic acid);
8,8′-(carbonyl-bis(imino-N-methyl-4,2-pyrrolecarbonylimino(N-methyl-4,2-pyrrole)carbonylimino))bis(1,3-naphthalenedisulfonic acid);
8,8′-(carbonyl-bis(imino-N-methyl-4,2-pyrrolecarbonylimino(N-methyl-4,2-pyrrole)carbonylimino))bis(3,5-naphthalenedisulfonic acid);
8,8′-(carbonyl-bis(imino-N-methyl-4,2-pyrrolecarbonylimino(N-methyl-4,2-pyrrole)carbonylimino))bis(1,5-naphthalenedisulfonic acid);
8,8′-(carbonyl-bis(imino-N-methyl-4,2-pyrrolecarbonylimino(N-methyl-4,2-pyrrole)carbonylimino))bis(3-naphthalenesulfonic acid);
8,8′-(carbonyl-bis(imino-N-methyl-4,2-pyrrolecarbonylimino(N-methyl-4,2-pyrrole)carbonylimino))bis(1-naphthalenesulfonic acid);
7,7′-(carbonyl-bis(imino-N-methyl-4,2-pyrrolecarbonylimino(N-methyl-4,2-pyrrole)carbonylimino))bis(1,6-naphthalenedisulfonic acid);
7,7′-(carbonyl-bis(imino-N-methyl-4,2-pyrrolecarbonylimino(N-methyl-4,2-pyrrole)carbonylimino))bis(2,6-naphthalenedisulfonic acid);
7,7′-(carbonyl-bis(imino-N-methyl-4,2-pyrrolecarbonylimino(N-methyl-4,2-pyrrole)carbonylimino))bis(1,5-naphthalenedisulfonic acid);
7,7′-(carbonyl-bis(imino-N-methyl-4,2-pyrrolecarbonylimino(N-methyl-4,2-pyrrole)carbonylimino))bis(2,5-naphthalenedisulfonic acid);
7,7′-(carbonyl-bis(imino-N-methyl-4,2-pyrrolecarbonylimino(N-methyl-4,2-pyrrole)carbonylimino))bis(2,3-naphthalenedisulfonic acid);
8,8′-(carbonyl-bis(imino-N-methyl-4,2-pyrrolecarbonylimino(N-methyl-4,2-pyrrole)carbonylimino))bis(1,6-naphthalenedisulfonic acid);
8,8′-(carbonyl-bis(imino-N-methyl-4,2-pyrrolecarbonylimino(N-methyl-4,2-pyrrole)carbonylimino))bis(2,6-naphthalenedisulfonic acid);
8,8′-(carbonyl-bis(imino-N-methyl-4,2-pyrrolecarbonylimino(N-methyl-4,2-pyrrole)carbonylimino))bis(2,5-naphthalenedisulfonic acid);
8,8′-(carbonyl-bis(imino-N-methyl-4,2-pyrrolecarbonylimino(N-methyl-4,2-pyrrole)carbonylimino))bis(3,6-naphthalenedisulfonic acid);
8,8′-(carbonyl-bis(imino-N-methyl-4,2-pyrrolecarbonylimino(N-methyl-4,2-pyrrole)carbonylimino))bis(2,3,5-naphthalenetrisulfonic acid);
8,8′-(carbonyl-bis(imino-N-methyl-4,2-pyrrolecarbonylimino(N-methyl-4,2-pyrrole)carbonylimino))bis(1,4,6-naphthalenetrisulfonic acid);
8,8′-(carbonyl-bis(imino-N-methyl-4,2-pyrrolecarbonylimino(N-methyl-4,2-pyrrole)carbonylimino))bis(2,4,6-naphthalenetrisulfonic acid);
7,7′-(carbonyl-bis(imino-N-methyl-4,2-pyrrolecarbonylimino(N-methyl-4,2-pyrrole)carbonylimino))bis(1-naphthalenesulfonic acid);
7,7′-(carbonyl-bis(imino-N-methyl-4,2-pyrrolecarbonylimino(N-methyl-4,2-pyrrole)carbonylimino))bis(2-naphthalenesulfonic acid);
7,7′-(carbonyl-bis(imino-N-methyl-4,2-pyrrolecarbonylimino(N-methyl-4,2-pyrrole)carbonylimino))bis(3-naphthalenesulfonic acid);
7,7′-(carbonyl-bis(imino-N-methyl-4,2-pyrrolecarbonylimino(N-methyl-4,2-pyrrole)carbonylimino))bis(4-naphthalenesulfonic acid);
7,7′-(carbonyl-bis(imino-N-methyl-4,2-pyrrolecarbonylimino(N-methyl-4,2-pyrrole)carbonylimino))bis(1,4,6-naphthalenetrisulfonic acid);
7,7′-(carbonyl-bis(imino-N-methyl-4,2-pyrrolecarbonylimino(N-methyl-4,2-pyrrole)carbonylimino))bis(1,3,6-naphthalenetrisulfonic acid);
7,7′-(carbonyl-bis(imino-N-methyl-4,2-pyrrolecarbonylimino))bis(1,3-naphtha lenedisulfonic acid);
7,7′-(carbonyl-bis(imino-N-methyl-4,2-pyrrolecarbonylimino(N-methyl-4,2-pyrrolecarbonylimino(N-methyl-4,2-pyrrole)carbonylimino))bis(1,3-naphthalenedisulfonicacid);
7,7′-(carbonyl-bis(imino-N-methyl-4,2-pyrrolecarbonylimino(N-methyl-4,2-pyrrole)carbonylimino))bis(2,4,6-naphthalenetrisulfonic acid); and
7,7′-(carbonyl-bis(imino-N-methyl-4,2-pyrrolecarbonylimino(N-methyl-4,2-pyrrole)carbonylimino))bis(2,3,5-naphthalenetrisulfonic acid).

In various embodiments, the PSMA ligands represented by variable A in Structural Formulas A1, A2, A3, A11, A12, and A14 preferred compounds of this embodiment can be selected from the group consisting of:

suramin;
8,8′-[carbonylbis(imino-3,1-phenylene)]bis-(1,3,5-naphthalenetrisulfonic acid);
5-[[[1-methyl-4-[[[[1-methyl-6-[[(4,5,7-trisulfo-1-naphthalenyl)amino]carbonyl]-1H-pyrrol-3-yl]amino]carbonyl]amino]-1H-pyrrol-2-yl]carbonyl]amino]1,3,8-naphthalenetrisulfonic acid; and
4-[[[1-methyl-4-[[[[1-methyl-5-[[(4-sulfo-1-naphthalenyl)amino]carbonyl]-1H-pyrrol-3-yl]amino]carbonyl]amino]-1H-pyrrol-2-yl]carbonyl]amino]-1-naphtha lenesulfonic acid.

### PMSA Ligands (D.1)

In various embodiments, the PSMA ligands represented by variable A in Structural Formulas A1, A2, A3, A11, A12, and A14 can be those described in U.S. Pat. No. 6,025,345, or the patents and applications to which it claims priority, U.S. Pat. Nos. 5,804,602, 5,672,592, and U.S. patent application Ser. No. 08/864,545, filed May 28, 1997 and 08/863,624, filed May 27, 1997. The entire teachings of each of these documents are incorporated herein by reference. The variables and terms in this section can be as described herein, more typically as described in this section, or in preferred embodiments can be as described in the documents incorporated by reference in this paragraph.

In various embodiments, the PSMA ligands represented by variable A in Structural Formulas A1, A2, A3, A11, A12, and A14 can be represented by the following Structural Formula:

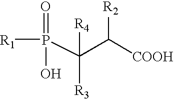

wherein:

R₁ is hydrogen, C₁-C₉ straight or branched chain alkyl, C₂-C₉ straight or branched chain alkenyl group, C₃-C₈ cycloalkyl, C₅-C₇ cycloalkenyl, or Ar₁;

R₂ is C₁-C₉ straight or branched chain alkyl, C₂-C₈ straight or branched chain alkenyl group, C₃-C₈ cycloalkyl, C₅-C₇ cycloalkenyl, or Ar₁, wherein said alkyl, alkenyl, cycloalkyl, cycloalkenyl or aryl groups may be optionally substituted with carboxylic acid;

R₃ and R₄ are independently hydrogen, C₁-C₆ straight or branched chain alkyl, C₂-C₆ straight or branched chain alkenyl, dialkyl, halogen, or Ar₁ provided that both R₃ and R₄ are not hydrogen.

Preferred compounds can be those wherein R₁ is either a straight or branched aliphatic group or a carbocyclic group, and R₂ is ethyl which is substituted with a carboxylic acid.

In various embodiments, the PSMA ligands represented by variable A in Structural Formulas A1, A2, A3, A11, A12, and A14 can be selected from the group consisting of:
2-[1-[methylhydroxyphosphinyl]ethyl]pentanedioic acid;
2-[1-[ethylhydroxyphosphinyl]propyl]pentanedioic acid;
2-[1-[propylhydroxyphosphinyl]butyl]pentanedioic acid;
2-[1-[butylhydroxyphosphinyl]but-2-enyl]pentanedioic acid;
2-[1-[cyclohexylhydroxyphosphinyl]pentyl]pentanedioic acid;
2-[1-[(cyclohexyl)methylhydroxyphosphinyl]hexyl]pentanedioic acid;
2-[1-[phenylhydroxyphosphinyl]heptyl]pentanedioic acid;
2-[1-[phenylhydroxyphosphinyl]-1-fluoromethyl]pentanedioic acid;
2-[2-[benzylhydroxyphosphinyl]propyl]pentanedioic acid;
2-[1-[benzylhydroxyphosphinyl]-1-phenylmethyl]pentanedioic acid;
2-[1-[phenylethylhydroxyphosphinyl]ethyl]pentanedioic acid;
2-[1-[phenylpropylhydroxyphosphinyl]propyl]pentanedioic acid;
2-[1-[phenylbutylhydroxyphosphinyl]butyl]pentanedioic acid;
2-[1-[(4-methylbenzyl)hydroxyphosphinyl]but-3-enyl]pentanedioic acid;
2-[1-[(4-fluorobenzyl)hydroxyphosphinyl]pentyl]pentanedioic acid;
2-[1-[(2-fluorobenzyl)hydroxyphosphinyl]hexyl]pentanedioic acid;
2-[1-[(pentafluorobenzyl)hydroxyphosphinyl]heptyl]pentanedioic acid;
2-[2-[(methoxybenzyl)hydroxyphosphinyl]butyl]pentanedioic acid;
2-[[(4-fluorophenyl)hydroxyphosphinyl]ethyl]pentanedioic acid;
2-[1-[((hydroxy)phenylmethyl)hydroxyphosphinyl]propyl]pentanedioic acid;
2-[1-[(3-methylbenzyl)hydroxyphosphinyl]butyl]pentanedioic acid;
2-(1-phosphonobut-2-enyl)pentanedioic acid;
2-[1-[(3-trifluoromethylbenzyl)hydroxyphosphinyl]pentyl]pentanedioic acid;
2-[1-[(2,3,4-trimethoxyphenyl)hydroxyphosphinyl]hexyl]pentanedioic acid;
2-[1-[(1-naphthyl)hydroxyphosphinyl]heptyl]pentanedioic acid;
2-[1-[(1-naphthyl)hydroxyphosphinyl]-1-fluoromethyl]pentanedioic acid;
2-[2-[(2-naphthyl)hydroxyphosphinyl]butyl]pentanedioic acid;
2-[1-[(2-naphthyl)hydroxyphosphinyl]-1-phenylmethyl]pentanedioic acid;
2-[1-[(1-naphthyl)methylhydroxyphosphinyl]ethyl]pentanedioic acid;
2-[1-[(2-naphthyl)methylhydroxyphosphinyl]propyl]pentanedioic acid;
2-[1-[(1-naphthyl)ethylhydroxyphosphinyl]butyl]pentanedioic acid;
2-[1-[(2-naphthyl)ethylhydroxyphosphinyl]but-3-enyl]pentanedioic acid;
2-[1-[(1-naphthyl)propylhydroxyphosphinyl]pentyl]pentanedioic acid;
2-[1-[(2-naphthyl)propylhydroxyphosphinyl]hexyl]pentanedioic acid;
2-[1-[(1-naphthyl)butylhydroxyphosphinyl]heptyl]pentanedioic acid;
2-[2-[(2-naphthyl)butylhydroxyphosphinyl]pentyl]pentanedioic acid; and
2-[1-[(phenylprop-2-enyl)hydroxyphosphinyl]ethyl]pentanedioic acid.

In various embodiments, the PSMA ligands represented by variable A in Structural Formulas A1, A2, A3, A11, A12, and A14 can be selected from the group consisting of:
2-[1-(benzylhydroxyphosphinyl)propyl]pentanedioic acid;
2-[1-(phenylhydroxyphosphinyl)butyl]pentanedioic acid;
2-[1-[((hydroxy)phenylmethyl)hydroxyphosphinyl]but-2-enyl]pentanedioic acid;
2-[1-(butylhydroxyphosphinyl)pentyl]pentanedioic acid;
2-[1-[(3-methylbenzyl)hydroxyphosphinyl]hexyl]pentanedioic acid;
2-[1-(3-phenylpropylhydroxyphosphinyl)heptyl]pentanedioic acid;
2-[1-(3-phenylpropylhydroxyphosphinyl)-1-fluoromethyl]pentanedioic acid;
2-[2-[(4-fluorophenyl)hydroxyphosphinyl]pent-3-enyl]pentanedioic acid;
2-[1-[(4-fluorophenyl)hydroxyphosphinyl]-1-phenylmethyl]pentanedioic acid;
2-[1-(methylhydroxyphosphinyl)ethyl]pentanedioic acid;
2-[1-(phenylethylhydroxyphosphinyl)propyl]pentanedioic acid;
2-[1-[(4-methylbenzyl)hydroxyphosphinyl]butyl]pentanedioic acid;
2-[1-[(4-fluorobenzyl)hydroxyphosphinyl]but-3-enyl]pentanedioic acid;
2-[1-[(4-methoxybenzyl)hydroxyphosphinyl]pentyl]pentanedioic acid;
2-[1-[(2-fluorobenzyl)hydroxyphosphinyl]hexyl]pentanedioic acid;
2-[1-[(pentafluorobenzyl)hydroxyphosphinyl]heptyl]pentanedioic acid;
2-(2-phosphonopent-4-enyl)pentanedioic acid; and
2-[1-[(3-trifluoromethylbenzyl)hydroxyphosphinyl]ethyl]pentanedioic acid.

In various embodiments, the PSMA ligands represented by variable A in Structural Formulas A1, A2, A3, A11, A12, and A14 can be selected from the group consisting of:
3-(methylhydroxyphosphinyl)-3-ethyl-2-phenylpropanoic acid;
3-(ethylhydroxyphosphinyl)-3-propyl-2-phenylpropanoic acid;
3-(propylhydroxyphosphinyl)-3-prop-2-enyl-2-phenylpropanoic acid;
3-(butylhydroxyphosphinyl)-3-t-butyl-2-phenylpropanoic acid;
3-(cyclohexylhydroxyphosphinyl)-3-pentyl-2-phenylpropanoic acid;
3-((cyclohexyl)methylhydroxyphosphinyl)-3-hexyl-2-phenylpropanoic acid;
3-((cyclohexyl)methylhydroxyphosphinyl)-3-fluoro-2-phenylpropanoic acid;
3-(phenylhydroxyphosphinyl)-3-methyl-3-butyl-2-phenylpropanoic acid;
3-(phenylhydroxyphosphinyl)-2,3-diphenylpropanoic acid;
3-(benzylhydroxyphosphinyl)-3-methyl-2-phenylpropanoic acid;
3-(phenylethylhydroxyphosphinyl)-3-ethyl-2-phenylpropanoic acid;
3-(phenylpropylhydroxyphosphinyl)-3-propyl-2-phenylpropanoic acid;
3-(phenylbutylhydroxyphosphinyl)-3-prop-1-enyl-2-phenylpropanoic acid;
3-[(2,3,4-trimethoxyphenyl)-3-hydroxyphosphinyl]-3-t-butyl-2-phenylpropanoic acid;
3-[(1-naphthyl)hydroxyphosphinyl]-3-pentyl-2-phenylpropanoic acid;
3-[(2-naphthyl)hydroxyphosphinyl]-3-hexyl-2-phenylpropanoic acid;
3-[(1-naphthyl)methylhydroxyphosphinyl]-3-methyl-3-pentyl-2-phenylpropanoic acid;
3-[(2-naphthyl)methylhydroxyphosphinyl]-3-methyl-2-phenylpropanoic acid;
3-[(1-naphthyl)ethylhydroxyphosphinyl]-3-ethyl-2-phenylpropanoic acid;
3-[(2-naphthyl)ethylhydroxyphosphinyl]-3-propyl-2-phenylpropanoic acid;
3-[(1-naphthyl)propylhydroxyphosphinyl]-3-prop-2-enyl-2-phenylpropanoic acid;
3-[(2-naphthyl)propylhydroxyphosphinyl]-3-t-butyl-2-phenylpropanoic acid;
3-[(1-naphthyl)butylhydroxyphosphinyl]-3-pentyl-2-phenylpropanoic acid;
3-[(2-naphthyl)butylhydroxyphosphinyl]-3-hexyl-2-phenylpropanoic acid; and
3-[phenylprop-2-enylhydroxyphosphinyl]-3-methyl-3-hexyl-2-phenylpropanoic acid.

In various embodiments, the PSMA ligand represented by variable A in Structural Formulas A1, A2, A3, A11, A12, and A14 is 2-[1-[benzylhydroxyphosphinyl]ethyl]pentanedioic acid.

Other preferred PSMA ligands of this section can be selected from the group consisting of: hydroxyphosphinyl derivatives wherein, R₁ is a straight or branched aliphatic group or a carbocyclic group and R₂ is an C₂-C₈ alkyl or alkenyl chain which is substituted with a carboxylic acid. Exemplary species include:
2-[1-(methylhydroxyphosphinyl)propyl]hexanedioic acid;
2-[1-(benzylhydroxyphosphinyl)butyl]hexanedioic acid;
2-[1-(methylhydroxyphosphinyl)but-2-enyl]heptanedioic acid;
2-[1-(benzylhydroxyphosphinyl)pentyl]heptanedioic acid;
2-[1-(methylhydroxyphosphinyl)hexyl]octanedioic acid;
2-[1-(benzylhydroxyphosphinyl)heptyl]octanedioic acid;
2-[1-(benzylhydroxyphosphinyl)-1-fluoromethyl]octanedioic acid;
2-[3-(methylhydroxyphosphinyl)pentyl]nonanedioic acid;
2-[1-(methylhydroxyphosphinyl)-1-phenylmethyl]nonanedioic acid;
2-[1-(benzylhydroxyphosphinyl)ethyl]nonanedioic acid;
2-[1-(methylhydroxyphosphinyl)propyl]decanedioic acid; and
2-[1-(benzylhydroxyphosphinyl)butyl]decanedioic acid.

Other preferred PSMA ligands can be selected from the group consisting of: hydroxyphosphinyl derivatives wherein R₁ is benzyl and R₂ is a straight or branched aliphatic group or a carbocyclic group. Exemplary species include:
3-(benzylhydroxyphosphinyl)-3-prop-2-enyl-2-methylpropanoic acid;
3-(benzylhydroxyphosphinyl)-3-t-butyl-2-ethylpropanoic acid;
3-(benzylhydroxyphosphinyl)-3-pentyl-2-propylpropanoic acid;
3-(benzylhydroxyphosphinyl)-3-hexyl-2-butylpropanoic acid;
3-(benzylhydroxyphosphinyl)-3-fluoro-2-butylpropanoic acid;
3-(benzylhydroxyphosphinyl)-3-ethyl-3-propyl-2-cyclohexylpropanoic acid;
3-(benzylhydroxyphosphinyl)-3-phenyl-2-cyclohexylpropanoic acid;
3-(benzylhydroxyphosphinyl)-3-methyl-2-(cyclohexyl)methylpropanoic acid;
3-(benzylhydroxyphosphinyl)-3-ethyl-2-phenylpropanoic acid;
3-(benzylhydroxyphosphinyl)-3-propyl-2-benzylpropanoic acid;
3-(benzylhydroxyphosphinyl)-3-propyl-enyl-2-phenylethylpropanoic acid;
3-(benzylhydroxyphosphinyl)-3-t-butyl-2-phenylpropylpropanoic acid;
3-(benzylhydroxyphosphinyl)-3-pentyl-2-phenylbutylpropanoic acid;
3-(benzylhydroxyphosphinyl)-3-hexyl-2-(2,3,4-trimethoxyphenyl)propanoic acid;
3-(benzylhydroxyphosphinyl)-3-ethyl-3-prop-1-enyl-2-(1-naphthyl)propanoic acid;
3-(benzylhydroxyphosphinyl)-3-methyl-2-(2-naphthyl)propanoic acid;
3-(benzylhydroxyphosphinyl)-3-ethyl-2-(1-naphthyl)methyl propanoic acid;
3-(benzylhydroxyphosphinyl)-3-propyl-2-(2-naphthyl)methyl propanoic acid;
3-(benzylhydroxyphosphinyl)-3-prop-2-enyl-2-(1-naphthyl)ethyl propanoic acid;
3-(benzylhydroxyphosphinyl)-3-t-butyl-2-(2-naphthyl)ethyl propanoic acid;
3-(benzylhydroxyphosphinyl)-3-pentyl-2-(1-naphthyl)propyl propanoic acid;
3-(benzylhydroxyphosphinyl)-3-hexyl-2-(2-naphthyl)propyl propanoic acid;
3-(benzylhydroxyphosphinyl)-3-fluoro-2-(2-naphthyl)propyl propanoic acid;
3-(benzylhydroxyphosphinyl)-3-ethyl-3-butyl-2-(1-naphthyl) butylpropanoic acid;
3-(benzylhydroxyphosphinyl)-3-phenyl-2-(1-naphthyl)butyl propanoic acid;
3-(benzylhydroxyphosphinyl)-3-methyl-2-(2-naphthyl)butyl propanoic acid; and
3-(benzylhydroxyphosphinyl)-3-ethyl-2-phenylprop-2-enyl propanoic acid.

Other preferred PSMA ligands of this section can be are those wherein R₁ is said alkyl, alkenyl, cycloalkyl, or aryl group which is substituted with a heterocyclic group and R₂ is ethyl which is substituted with a carboxylic acid can be selected from the group consisting of:
2-[1-[(2-pyridyl)methylhydroxyphosphinyl]butyl]pentanedioic acid;
2-[1-[(3-pyridyl)methylhydroxyphosphinyl]but-2-enyl]pentanedioic acid;
2-[1-[(4-pyridyl)methylhydroxyphosphinyl]pentyl]pentanedioic acid;
2-[1-[(3-pyridyl)ethylhydroxyphosphinyl]hexyl]pentanedioic acid;
2-[1-[(3-pyridyl)propylhydroxyphosphinyl]heptyl]pentanedioic acid;
2-[1-[(3-pyridyl)propylhydroxyphosphinyl]-1-fluoromethyl]pentanedioic acid;
2-[3-[(tetrahydrofuranyl)methylhydroxyphosphinyl]octyl]pentanedioic acid;
2-[1-[(tetrahydrofuranyl)methylhydroxyphosphinyl]-1-phenylmethyl]pentanedioic acid;
2-[1-[(tetrahydrofuranyl)ethylhydroxyphosphinyl]ethyl]pentanedioic acid;
2-[1-[(tetrahydrofuranyl)propylhydroxyphosphinyl]propyl]pentanedioic acid;
2-[1-[(2-indolyl)methylhydroxyphosphinyl]butyl]pentanedioic acid;
2-[1-[(3-indolyl)methylhydroxyphosphinyl]but-3-enyl]pentanedioic acid;
2-[1-[(4-indolyl)methylhydroxyphosphinyl]pentyl]pentanedioic acid;
2-[1-[(3-indolyl)ethylhydroxyphosphinyl]hexyl]pentanedioic acid;
2-[1-[(3-indolyl)propylhydroxyphosphinyl]heptyl]pentanedioic acid;
2-[3-[(2-thienyl)methylhydroxyphosphinyl]nonyl]pentanedioic acid;
2-[1-[(3-thienyl)methylhydroxyphosphinyl]ethyl]pentanedioic acid;
2-[1-[(4-thienyl)methylhydroxyphosphinyl]propyl]pentanedioic acid;
2-[1-[(3-thienyl)ethylhydroxyphosphinyl]butyl]pentanedioic acid; and
2-[1-[(3-thienyl)propylhydroxyphosphinyl]but-2-enyl]pentanedioic acid.

In various embodiments, the PSMA ligands represented by variable A in Structural Formulas A1, A2, A3, A11, A12, and A14 can be selected from the group consisting of:
3-[(2-pyridyl)methylhydroxyphosphinyl]-3-t-butyl-2-phenylpropanoic acid;
3-[(3-pyridyl)methylhydroxyphosphinyl]-3-pentyl-2-phenylpropanoic acid;
3-[(4-pyridyl)methylhydroxyphosphinyl]-3-hexyl-2-phenylpropanoic acid;
3-[(4-pyridyl)methylhydroxyphosphinyl]-3-fluoro-2-phenylpropanoic acid;
3-[(3-pyridyl)ethylhydroxyphosphinyl]-3-dipropyl-2-phenylpropanoic acid;
3-[(3-pyridyl)ethylhydroxyphosphinyl]-2,3-diphenylpropanoic acid;
3-[(3-pyridyl)propylhydroxyphosphinyl]-3-methyl-2-phenylpropanoic acid;
3-[(tetrahydrofuranyl)methylhydroxyphosphinyl]-3-ethyl-2-phenylpropanoic acid;
3-[(tetrahydrofuranyl)ethylhydroxyphosphinyl]-3-propyl-2-phenylpropanoic acid;
3-[(tetrahydrofuranyl)propylhydroxyphosphinyl]-3-prop-2-enyl-2-phenylpropan oic acid;
3-[(2-indolyl)methylhydroxyphosphinyl]-3-t-butyl-2-phenylpropanoic acid;
3-[(3-indolyl)methylhydroxyphosphinyl]-3-pentyl-2-phenylpropanoic acid;
3-[(4-indolyl)methylhydroxyphosphinyl]-3-hexyl-2-phenylpropanoic acid;
3-[(3-indolyl)ethylhydroxyphosphinyl]-3-propyl-3-t-butyl-2-phenylpropanoic acid;
3-[(3-indolyl)propylhydroxyphosphinyl]-3-methyl-2-phenylpropanoic acid;
3-[(2-thienyl)methylhydroxyphosphinyl]-3-ethyl-2-phenylpropanoic acid;
3-[(3-thienyl)methylhydroxyphosphinyl]-3-propyl-2-phenylpropanoic acid;
3-[(4-thienyl)methylhydroxyphosphinyl]-3-prop-1-enyl-2-phenylpropanoic acid;
3-[(3-thienyl)ethylhydroxyphosphinyl]-3-t-butyl-2-phenylpropanoic acid; and
3-[(3-thienyl)propylhydroxyphosphinyl]-3-pentyl-2-phenylpropanoic acid.

Other preferred PSMA ligands of this section can be wherein R₁ is benzyl and R₂ is said alkyl, alkenyl, cycloalkyl, or aryl group which is substituted with a heterocyclic group can be selected from the group consisting of:
3-(benzylhydroxyphosphinyl)-3-hexyl-2-(2-pyridyl)methyl propanoic acid;
3-(benzylhydroxyphosphinyl)-3-fluoro-2-(2-pyridyl)methyl propanoic acid;
3-(benzylhydroxyphosphinyl)-3-propyl-3-pentyl-2-(3-pyridyl)methylpropanoic acid;
3-(benzylhydroxyphosphinyl)-3-phenyl-2-(3-pyridyl)methyl propanoic acid;
3-(benzylhydroxyphosphinyl)-3-methyl-2-(4-pyridyl)methyl propanoic acid;
3-(benzylhydroxyphosphinyl)-3-ethyl-2-(3-pyridyl)ethylpropanoic acid;
3-(benzylhydroxyphosphinyl)-3-propyl-2-(3-pyridyl)propyl propanoic acid;
3-(benzylhydroxyphosphinyl)-3-prop-2-enyl-2-(tetrahydrofuranyl)methylpropanoic acid;
3-(benzylhydroxyphosphinyl)-3-t-butyl-2-(tetrahydrofuranyl)ethylpropanoic acid;
3-(benzylhydroxyphosphinyl)-3-pentyl-2-(tetrahydrofuranyl)propylpropanoic acid;
3-(benzylhydroxyphosphinyl)-3-hexyl-2-(2-indolyl)methyl propanoic acid;
3-(benzylhydroxyphosphinyl)-3-propyl-3-hexyl-2-(3-indolyl)methylpropanoic acid;
3-(benzylhydroxyphosphinyl)-3-methyl-2-(4-indolyl)methyl propanoic acid;
3-(benzylhydroxyphosphinyl)-3-ethyl-2-(3-indolyl)ethyl propanoic acid;
3-(benzylhydroxyphosphinyl)-3-propyl-2-(3-indolyl)propyl propanoic acid;
3-(benzylhydroxyphosphinyl)-3-prop-1-enyl-2-(2-thienyl)methyl propanoic acid;
3-(benzylhydroxyphosphinyl)-3-t-butyl-2-(3-thienyl)methyl propanoic acid;
3-(benzylhydroxyphosphinyl)-3-pentyl-2-(4-thienyl)methyl propanoic acid;
3-(benzylhydroxyphosphinyl)-3-hexyl-2-(3-thienyl)ethyl propanoic acid; and
3-(benzylhydroxyphosphinyl)-3-t-butyl-3-pentyl-2-(3-thienyl)propylpropanoic acid.

In various embodiments, the PSMA ligands represented by variable A in Structural Formulas A1, A2, A3, A11, A12, and A14 can be represented by the following Structural Formula:

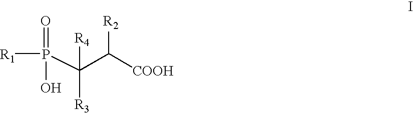

wherein:

R₁ is Ar₁;

R₂ is C₁-C₉ straight or branched chain alkyl, C₂-C₈ straight or branched chain alkenyl group, C₃-C₈ cycloalkyl, C₅-C₇ cycloalkenyl, or Ar₁, wherein said alkyl, alkenyl, cycloalkyl, cycloalkenyl or aryl group may be optionally substituted with carboxylic acid;

R₃ and R₄ are independently hydrogen, C₁-C₆ straight or branched chain alkyl, C₂-C₆ straight or branched chain alkenyl, dialkyl, halogen, or Ar₁, provided that both R₃ and R₄ are not hydrogen.

In various embodiments, the PSMA ligands represented by variable A in Structural Formulas A1, A2, A3, A11, A12, and A14 can be selected from the group consisting of:
2-[1-[(2-pyridyl)hydroxyphosphinyl]ethyl]pentanedioic acid;
2-[1-[(3-pyridyl)hydroxyphosphinyl]propyl]pentanedioic acid;
2-[1-[(4-pyridyl)hydroxyphosphinyl]butyl]pentanedioic acid;
2-[1-[(tetrahydrofuranyl)hydroxyphosphinyl]but-3-enyl]pentanedioic acid;
2-[1-[(2-indolyl)hydroxyphosphinyl]pentyl]pentanedioic acid;
2-[1-[(3-indolyl)hydroxyphosphinyl]hexyl]pentanedioic acid;
2-[1-[(4-indolyl)hydroxyphosphinyl]heptyl]pentanedioic acid;
2-[1-[(4-indolyl)hydroxyphosphinyl]-1-fluoromethyl]pentanedioic acid;
2-[2-[(2-thienyl)hydroxyphosphinyl]propyl]pentanedioic acid;
2-[1-[(2-thienyl)hydroxyphosphinyl]-1-phenylmethyl]pentanedioic acid;
2-[1-[(3-thienyl)hydroxyphosphinyl]ethyl]pentanedioic acid; and
2-[1-[(4-thienyl)hydroxyphosphinyl]propyl]pentanedioic acid.

PSMA ligands of this section wherein R₁ is a heterocyclic group and R₂ is phenyl can be selected from the group consisting of:
3-[(2-pyridyl)hydroxyphosphinyl]-3-prop-1-enyl-2-phenylpropanoic acid;
3-[(3-pyridyl)hydroxyphosphinyl]-3-t-butyl-2-phenylpropanoic acid;
3-[(4-pyridyl)hydroxyphosphinyl]-3-pentyl-2-phenylpropanoic acid;
3-[(tetrahydrofuranyl)hydroxyphosphinyl]-3-hexyl-2-phenylpropanoic acid;
3-[(tetrahydrofuranyl)hydroxyphosphinyl]-3-fluoro-2-phenylpropanoic acid;
3-[(2-indolyl)hydroxyphosphinyl]-3-t-butyl-3-hexyl-2-phenylpropanoic acid;
3-[(2-indolyl)hydroxyphosphinyl]-2,3-diphenylpropanoic acid;
3-[(3-indolyl)hydroxyphosphinyl]-3-methyl-2-phenylpropanoic acid;
3-[(4-indolyl)hydroxyphosphinyl]-3-ethyl-2-phenylpropanoic acid;
3-[(2-thienyl)hydroxyphosphinyl]-3-propyl-2-phenylpropanoic acid;
3-[(3-thienyl)hydroxyphosphinyl]-3-prop-2-enyl-2-phenylpropanoic acid; and
3-[(4-thienyl)hydroxyphosphinyl]-3-t-butyl-2-phenylpropanoic acid.

PSMA ligands of this section can also preferably selected from the group of formula I:

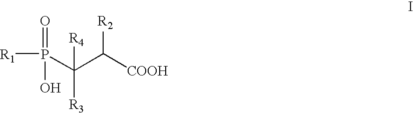

wherein R₁ is hydrogen, C₁-C₉ straight or branched chain alkyl, C₂-C₉ straight or branched chain alkenyl group, C₃-C₈ cycloalkyl, C₅-C₇ cycloalkenyl, or Ar₁;

R₂ is Ar₁, wherein said aryl group may be optionally substituted with carboxylic acid;

R₃ and R₄ are independently hydrogen, C₁-C₆ straight or branched chain alkyl, C₂-C₆ straight or branched chain alkenyl, dialkyl, halogen, or Ar₁, provided that both R₃ and R₄ are not hydrogen.

Particular embodiments can include species wherein R₂ is heterocyclic.

In various embodiments, the PSMA ligands represented by variable A in Structural Formulas A1, A2, A3, A11, A12, and A14 can be selected from the group consisting of:
3-(benzylhydroxyphosphinyl)-3-pentyl-2-(2-pyridyl)propanoic acid;
3-(benzylhydroxyphosphinyl)-3-hexyl-2-(3-pyridyl)propanoic acid;
3-(benzylhydroxyphosphinyl)-3-fluoro-2-(3-pyridyl)propanoic acid;
3-(benzylhydroxyphosphinyl)-3-pentyl-3-hexyl-2-(4-pyridyl) propanoic acid;
3-(benxylhydroxyphosphinyl)-3-phenyl-2-(4-pyridyl)propanoic acid;
3-(benzylhydroxyphosphinyl)-3-methyl-2-(tetrahydrofuranyl) propanoic acid;
3-(benzylhydroxyphosphinyl)-3-ethyl-2-(2-indolyl)propanoic acid;
3-(benzylhydroxyphosphinyl)-3-propyl-2-(3-indolyl)propanoic acid;
3-(benzylhydroxyphosphinyl)-3-prop-1-enyl-2-(4-indolyl) propanoic acid;
3-(benzylhydroxyphosphinyl)-3-t-butyl-2-(2-thienyl)propanoic acid;
3-(benzylhydroxyphosphinyl)-3-pentyl-2-(3-thienyl)propanoic acid; and
3-(benzylhydroxyphosphinyl)-3-hexyl-2-(4-thienyl)propanoic acid.

### PMSA Ligands (D.2)

In various embodiments, the PSMA ligands represented by variable A in Structural Formulas A1, A2, A3, A11, A12, and A14 can be those described in U.S. Pat. No. 6,054,444. The entire teachings of this documents are incorporated herein by reference. The variables and terms in this section can be as described herein, more typically as described in this section, or in preferred embodiments can be as described in the documents incorporated by reference in this paragraph.

In various embodiments, the PSMA ligands represented by variable A in Structural Formulas A1, A2, A3, A11, A12, and A14 can be represented by the following Structural Formula:

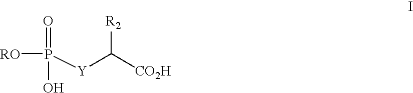

wherein:

Y is selected from the group consisting of CR₁R₂, NR₃ and O;

X is selected from the group consisting of hydrogen, C₁-C₉ straight or branched chain alkyl, C₂-C₉ straight or branched chain alkenyl, C₃-C₈ cycloalkyl, C₅-C₇ cycloalkenyl and Ar, wherein said X is unsubstituted or substituted with carboxy, C₃-C₈ cycloalkyl, C₅-C₇ cycloalkenyl, halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, amino, Ar or mixtures thereof;

R, R₁, R₂ and R₃ are independently selected from the group consisting of hydrogen, C₁-C₉ straight or branched chain alkyl, C₂-C₉ straight or branched chain alkenyl, C₃-C₈ cycloalkyl, C₅-C₇ cycloalkenyl and Ar, wherein said R, R₁, R₂ and R₃ are independently unsubstituted or substituted with C₃-C₈ cycloalkyl, C₅-C₇ cycloalkenyl, halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, amino, Ar or mixtures thereof, provided that R is not hydrogen when X is hydrogen, carboxymethyl or carboxyethyl; and

Ar is selected from the group consisting of 1-naphthyl, 2-naphthyl, 2-indolyl, 3-indolyl, 2-furyl, 3-furyl, 2-thienyl, 3-thienyl, 2-pyridyl, 3-pyridyl, 4-pyridyl, benzyl and phenyl, wherein said Ar has one to three substituent(s) independently selected from the group consisting of hydrogen, halo, hydroxy, nitro, trifluoromethyl, C₁-C₆ straight or branched chain alkyl, C₂-C₆, straight or branched chain alkenyl, C₁-C₄ alkoxy, C₂-C₄ alkenyloxy, phenoxy, benzyloxy, amino and mixtures thereof.

In various embodiments, the PSMA ligands represented by variable A in Structural Formulas A1, A2, A3, A11, A12, and A14 can be selected from the group consisting of:

phosphonopropanoic acid;
2-methyl-3-phosphonopropanoic acid;
2-ethyl-3-phosphonopropanoic acid;
2-propyl-3-phosphonopropanoic acid;
2-butyl-3-phosphonopropanoic acid;
2-phenyl-3-phosphonopropanoic acid;
2-(2-phenylethyl)-3-phosphonopropanoic acid;
2-(3-phenylpropyl)-3-phosphonopropanoic acid;
2-(4-pyridyl)-3-phosphonopropanoic acid;
2-benzyl-3-phosphonopropanoic acid;
2-O-(methylphosphonomethyl)pentanedioic acid;
2-O-(ethylphosphonomethyl)pentanedioic acid;
2-O-(propylphosphonomethyl)pentanedioic acid;
2-O-(butylphosphonomethyl)pentanedioic acid;
2-O-(phenylphosphonomethyl)pentanedioic acid;
2-O-[(2-phenylethyl)phosphonomethyl]pentanedioic acid;
2-O-[(3-phenylpropyl)phosphonomethyl]pentanedioic acid;
2-O-[(4-pyridyl)phosphonomethyl]pentanedioic acid; and
2-O-(benzylphosphonomethyl)pentanedioic acid.

### PMSA Ligands (D.3)

In various embodiments, the PSMA ligands represented by variable A in Structural Formulas A1, A2, A3, A11, A12, and A14 can be those described in U.S. Pat. No. 6,071,965, or the patents and applications to which it claims priority, U.S. Pat. Nos. 5,804,602; 5,824,662; 5,672,592; 5,795,877; 5,863,536, and U.S. patent application Ser. No. 08/864,545, filed May 28, 1997; 08/884,479, filed Jun. 27, 1997; 08/718,703, filed Sep. 27, 1996; 08/842,360, filed Apr. 24, 1997; 08/863,624, filed May 27, 1997; 08/858,985, filed May 27, 1997; 08/899,319, filed Jul. 23, 1997; 08/835,572, filed Apr. 9, 1997; 08/900,194, filed Jul. 29, 1997; 08/665,775, filed Jun. 17, 1996; and 08/665,776, filed Jun. 17, 1996. The entire teachings of each of these documents are incorporated herein by reference. The variables and terms in this section can be as described herein, more typically as described in this section, or in preferred embodiments can be as described in the documents incorporated by reference in this paragraph.

In various embodiments, the PSMA ligands represented by variable A in Structural Formulas A1, A2, A3, A11, A12, and A14 can be represented by the following Structural Formula:

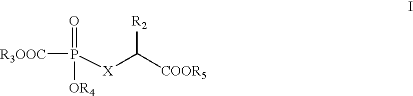

wherein:

X is CR₆R₇, O or NR₈;

R₁ is selected from the group consisting of C₁-C₉ straight or branched chain alkyl, C₂-C₉ straight or branched chain alkenyl, C₃-C₈ cycloalkyl, C₅-C₇ cycloalkenyl and Ar, wherein said R₁ is unsubstituted or substituted with one or more substituent(s) independently selected from the group consisting of carboxy, carbonyl, C₃-C₈ cycloalkyl, C₅-C₇ cycloalkenyl, halo, hydroxy, nitro, trifluoromethyl, C₁-C₆ straight or branched chain alkyl, C₂-C₆ straight or branched chain alkenyl, C₁-C₉ alkoxy, C₂-C₈ alkenyloxy, phenoxy, benzyloxy, amino, and Ar;

R₂, R₃, R₄, R₅, R₆, R₇, and R₈ are independently selected from the group consisting of hydrogen, C₁-C₉ straight or branched chain alkyl, C₂-C₉ straight or branched chain alkenyl, C₃-C₈ cycloalkyl, C₅-C₇ cycloalkenyl and Ar, wherein said R₂, R₃, R₄, R₅, 6, R₇, and R₈ are independently unsubstituted or substituted with one or more substituent(s) independently selected from the group consisting of carboxy, carbonyl, C₃-C₇ cycloalkyl, C₅-C₇ cycloalkenyl, halo, hydroxy, nitro, trifluoromethyl, C₁-C₆ straight or branched chain alkyl, C₂-C₆ straight or branched chain alkenyl, C₁-C₉ alkoxy, C₂-C₉ alkenyloxy, phenoxy, benzyloxy, amino, and Ar; and

Ar is selected from the group consisting of 1-naphthyl, 2-naphthyl, 2-indolyl, 3-indolyl, 4-indolyl, 2-furyl, 3-furyl, tetrahydrofuranyl, tetrahydropyranyl, 2-thienyl, 3-thienyl, 2-pyridyl, 3-pyridyl, 4-pyridyl, benzyl and phenyl, wherein said Ar is unsubstituted or substituted with one or more substituent (s) independently selected from the group consisting of carboxy, carbonyl, halo, hydroxy, nitro, trifluoromethyl, C₁-C₆ straight or branched chain alkyl, C₂-C₆ straight or branched chain alkenyl, C₁-C₆ alkoxy, C₂-C₆ alkenyloxy, phenoxy, benzyloxy, and amino.

In various embodiments, the PSMA ligands represented by variable A in Structural Formulas A1, A2, A3, A11, A12, and A14 can be selected from the group consisting of:
2-[[(2-carboxypropyl)hydroxyphosphinyl]methyl]pentanedioic acid;
2-[[(2-carboxybutyl)hydroxyphosphinyl]methyl]pentanedioic acid;
2-[[(2-carboxypentyl)hydroxyphosphinyl]methyl]pentanedioic acid;
2-[[(2-carboxy-3-phenylpropyl)hydroxyphosphinyl]methyl]pentanedioic acid;
2-[[(2-carboxy-3-naphthylpropyl)hydroxyphosphinyl]methyl]pentanedioic acid;
2-[[(2-carboxy-3-pyridylpropyl)hydroxyphosphinyl]methyl]pentanedioic acid;
2-[[(2-benzyloxycarbonyl)-3-phenylpropyl)hydroxyphosphinyl]methyl]pentanedioic acid;
2-[[(2-methoxycarbonyl)-3-phenylpropyl)hydroxyphosphinyl]methyl]pentanedioic acid;
2-[[(3-carboxy-2-methoxycarbonyl)propyl)hydroxyphosphinyl]methyl]pentanedioic acid; and
2-[[(4-carboxy-2-methoxycarbonyl)butyl)hydroxyphosphinyl]methyl]pentanedioic acid.

### PMSA Ligands (D.4)

In various embodiments, the PSMA ligands represented by variable A in Structural Formulas A1, A2, A3, A11, A12, and A14 can be those described in U.S. Pat. No. 6,121,252. The entire teachings of this document are incorporated herein by reference. The variables and terms in this section can be as described herein, more typically as described in this section, or in preferred embodiments can be as described in the documents incorporated by reference in this paragraph.

In various embodiments, the PSMA ligands represented by variable A in Structural Formulas A1, A2, A3, A11, A12, and A14 can be represented by the following Structural Formula:

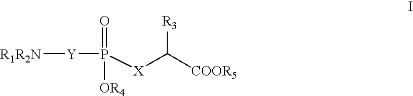

wherein:

X is CR₆R₇, O, or NR₈;

Y is C₁-C₉ straight or branched chain alkyl, C₂-C₉ straight or branched chain alkenyl, C₃-C₈ cycloalkyl, C₅-C₇ cycloalkenyl, or Ar₁, wherein Y is unsubstituted or substituted with one or more substituent(s);

R₁ and R₂ are independently selected from the group consisting of hydrogen, C₁-C₉ straight or branched chain alkyl, C₂-C₉ straight or branched chain alkenyl, C₃-C₈ cycloalkyl, C₅-C₇ cycloalkenyl, Ar₂, carboxy, carbonyl, sulfonyl, formanilido, and thioformamido, wherein R₁ and R₂ are independently unsubstituted or substituted with one or more substituent(s); or

R₁ and R₂ are taken together, with the nitrogen atom to which they are attached, to form a 5-7 membered heterocyclic ring, wherein said heterocyclic ring optionally contains one or more additional heteroatom(s) independently selected from the group consisting of N, O, and S, and said heterocyclic ring is unsubstituted or substituted with one or more substituent(s);

R₃, R₄, R₅, R₆, R₇, and R₈ are independently selected from the group consisting of hydrogen, C₁-C₉ straight or branched chain alkyl, C₂-C₉ straight or branched chain alkenyl, C₃-C₈ cycloalkyl, C₅-C₇ cycloalkenyl, and Ar₃, wherein R₃, R₄, R₅, R₆, R₇, and R₈ are independently unsubstituted or substituted with one or more substituent(s); and

Ar₁, Ar₂, and Ar₃ are independently a carbocyclic or heterocyclic moiety, which is unsubstituted or substituted with one or more substituent(s).

In a preferred embodiment, when X is CH₂ and Y is an unsubstituted or a monosubstituted CH₂, R₁C₁-C₉ straight or branched chain alkyl, C₂-C₉ straight or branched chain alkenyl, C₃-C₈ cycloalkyl, C₅-C₇ cycloalkenyl, or Ar₂, wherein R₁ is unsubstituted or substituted with one or more substituent(s).

In various embodiments, the PSMA ligands represented by variable A in Structural Formulas A1, A2, A3, A11, A12, and A14 can be selected from the group consisting of:
2-[({[Benzylamino]benzyl}(hydroxyphosphinyl))methyl]pentanedioic acid;
2-[({[Carboxyamino]benzyl}(hydroxyphosphinyl))methyl]pentanedioic acid;
2-[({[Acetylamino]benzyl}(hydroxyphosphinyl))methyl]pentanedioic acid;
2-[({[Dibenzylamino]benzyl}(hydroxyphosphinyl))methyl]pentanedioic acid;
2-[({[Phenylamino]benzyl}(hydroxyphosphinyl))methyl]pentanedioic acid;
2-({[(Phenylcarboxamido)benzyl](hydroxyphosphinyl)}methyl)pentanedioic acid;
2-({[(Phenylsulfonamido)benzyl](hydroxyphosphinyl)}methyl)pentanedioic acid;
2-[({[(2-Fluorophenyl)amino]benzyl}(hydroxyphosphinyl))methyl]pentanedioic acid;
2-[({[(3-Fluorophenyl)amino]benzyl}(hydroxyphosphinyl))methyl]pentanedioic acid;
2-[({[(4-Fluorophenyl)amino]benzyl}(hydroxyphosphinyl))methyl]pentanedioic acid;
2-[({[(2-Chlorophenyl)amino]benzyl}(hydroxyphosphinyl))methyl]pentanedioic acid;
2-[({[(3-Chlorophenyl)amino]benzyl}(hydroxyphosphinyl))methyl]pentanedioic acid;
2-[({[(4-Chlorophenyl)amino]benzyl}(hydroxyphosphinyl))methyl]pentanedioic acid;
2-[({[(2-Methoxyphenyl)amino]benzyl}(hydroxyphosphinyl))methyl]pentanedioic acid;
2-[({[(3-Methoxyphenyl)amino]benzyl}(hydroxyphosphinyl))methyl]pentanedioic acid;
2-[({[(4-Methoxyphenyl)amino]benzyl}(hydroxyphosphinyl))methyl]pentanedioic acid;
2-[({[(2-Hydroxyphenyl)amino]benzyl}(hydroxyphosphinyl))methyl]pentanedioic acid;
2-[({[(3-Hydroxyphenyl)amino]benzyl}(hydroxyphosphinyl))methyl]pentanedioic acid;
2-[({[(4-Hydroxyphenyl)amino]benzyl}(hydroxyphosphinyl))methyl]pentanedioic acid;
2-[({[(2-Carboxyphenyl)amino]benzyl}(hydroxyphosphinyl))methyl]pentanedioic acid;
2-[({[(3-Carboxyphenyl)amino]benzyl}(hydroxyphosphinyl))methyl]pentanedioic acid;
2-[({[(4-Carboxyphenyl)amino]benzyl}(hydroxyphosphinyl))methyl]pentanedioic acid;
2-[({[(2-Nitrophenyl)amino]benzyl}(hydroxyphosphinyl))methyl]pentanedioic acid;
2-[({[(3-Nitrophenyl)amino]benzyl}(hydroxyphosphinyl))methyl]pentanedioic acid;
2-[({[(4-Nitrophenyl)amino]benzyl}(hydroxyphosphinyl))methyl]pentanedioic acid;
2-[({[(2-Sulfonylphenyl)amino]benzyl}(hydroxyphosphinyl))methyl]pentanedioic acid;
2-[({[(3-Sulfonylphenyl)amino]benzyl}(hydroxyphosphinyl))methyl]pentanedioic acid;
2-[({[(4-Sulfonylphenyl)amino]benzyl}(hydroxyphosphinyl))methyl]pentanedioic acid;
2-[({[(2-Methylphenyl)amino]benzyl}(hydroxyphosphinyl))methyl]pentanedioic acid;
2-[({[(3-Methylphenyl)amino]benzyl}(hydroxyphosphinyl))methyl]pentanedioic acid;
2-[({[(4-Methylphenyl)amino]benzyl}(hydroxyphosphinyl))methyl]pentanedioic acid;
2-[({[(2-Tert-butylphenyl)amino]benzyl}(hydroxyphosphinyl))methyl]pentanedioic acid;
2-[({[(3-Tert-butylphenyl)amino]benzyl}(hydroxyphosphinyl))methyl]pentanedioic acid;
2-[({[(4-Tert-butylphenyl)amino]benzyl}(hydroxyphosphinyl))methyl]pentanedioic acid;
2-[({[(2-Trifluoromethylphenyl)amino]benzyl}(hydroxyphosphinyl))methyl]pent anedioic acid;
2-[({(3-Trifluoromethylphenyl)amino]benzyl}(hydroxyphosphinyl))methyl]pentanedioic acid;
2-[({[(4-Trifluoromethylphenyl)amino]benzyl}(hydroxyphosphinyl))methyl]pent anedioic acid;
2-[({[(Thioformanilido)amino]benzyl}(hydroxyphosphinyl))methyl]pentanedioic acid;
2-[({[1,3-Dioxo-2,3-dihydro-1H-2-isoindolyl]benzyl}hydroxyphosphinyl)methyl]pentanedioic acid;
2-[({[Benzylamino]methyl}(hydroxyphosphinyl))methyl]pentanedioic acid;
2-[({[Carboxyamino]methyl}(hydroxyphosphinyl))methyl]pentanedioic acid;
2-[({[Acetylamino]methyl}(hydroxyphosphinyl))methyl]pentanedioic acid;
2-[({[Diphenylamino]methyl}(hydroxyphosphinyl))methyl]pentanedioic acid;
2-[({[Phenylamino]methyl}(hydroxyphosphinyl))methyl]pentanedioic acid;
2-({[(Phenylcarboxamido)methyl](hydroxyphosphinyl)}methyl)pentanedioic acid;
2-({[(Phenylsulfonamido)methyl](hydroxyphosphinyl)}methyl)pentanedioic acid;
2-[({[(2-Fluorophenyl)amino]methyl}(hydroxyphosphinyl))methyl]pentanedioic acid;
2-[({[(3-Fluorophenyl)amino]methyl}(hydroxyphosphinyl))methyl]pentanedioic acid;
2-[({[(4-Fluorophenyl)amino]methyl}(hydroxyphosphinyl))methyl]pentanedioic acid;
2-[({[(2-Chlorophenyl)amino]methyl}(hydroxyphosphinyl))methyl]pentanedioic acid;
2-[({[(3-Chlorophenyl)amino]methyl}(hydroxyphosphinyl))methyl]pentanedioic acid;
2-[({[(4-Chlorophenyl)amino]methyl}(hydroxyphosphinyl))methyl]pentanedioic acid;
2-[({[(2-Methoxyphenyl)amino]methyl}(hydroxyphosphinyl))methyl]pentanedioic acid;
2-[({[(3-Methoxyphenyl)amino]methyl}(hydroxyphosphinyl))methyl]pentanedioic acid;
2-[({[(4-Methoxyphenyl)amino]methyl}(hydroxyphosphinyl))methyl]pentanedioic acid;
2-[({[(2-Hydroxyphenyl)amino]methyl}(hydroxyphosphinyl))methyl]pentanedioic acid;
2-[({[(3-Hydroxyphenyl)amino]methyl}(hydroxyphosphinyl))methyl]pentanedioic acid;
2-[({[(4-Hydroxyphenyl)amino]methyl}(hydroxyphosphinyl))methyl]pentanedioic acid;
2-[({[(2-Carboxyphenyl)amino]methyl}(hydroxyphosphinyl))methyl]pentanedioic acid;
2-[({[(3-Carboxyphenyl)amino]methyl)}(hydroxyphosphinyl))methyl]pentanedioic acid;
2-[({[(4-Carboxyphenyl)amino]methyl}(hydroxyphosphinyl))methyl]pentanedioic acid;
2-[({[(2-Nitrophenyl)amino]methyl)(hydroxyphosphinyl))methyl]pentanedioic acid;
2-[({[(3-Nitrophenyl)amino]methyl)(hydroxyphosphinyl))methyl]pentanedioic acid;
2-[({[(4-Nitrophenyl)amino]methyl}(hydroxyphosphinyl))methyl]pentanedioic acid;
2-[({[(2-Sulfonylphenyl)amino]methyl}(hydroxyphosphinyl))methyl]pentanedioic acid;
2-[({[(3-Sulfonylphenyl)amino]methyl}(hydroxyphosphinyl))methyl]pentanedioic acid;
2-[({[(4-Sulfonylphenyl)amino]methyl}(hydroxyphosphinyl))methyl]pentanedioic acid;
2-[({[(2-Methylphenyl)amino]methyl)(hydroxyphosphinyl))methyl]pentanedioic acid;
2-[({[(3-Methyphenyl)amino]methyl}(hydroxyphosphinyl))methyl]pentanedioic acid;
2-[({[(4-Methylphenyl)amino]methyl}(hydroxyphosphinyl))methyl]pentanedioic acid;
2-[({[(2-Tert-butylphenyl)amino]methyl}(hydroxyphosphinyl))methyl]pentanedioic acid;
2-[({[(3-Tert-butylphenyl)amino]methyl}(hydroxyphosphinyl))methyl]pentanedioic acid;
2-[({[(4-Tert-butylphenyl)amino]methyl}(hydroxyphosphinyl))methyl]pentanedioic acid;
2-[({[(2-Trifluoromethylphenyl)amino]methyl}(hydroxyphosphinyl))methyl]pent anedioic acid;
2-[({[(3-Trifluoromethylphenyl)amino]methyl}(hydroxyphosphinyl))methyl]pent anedioic acid;
2-[({[(4-Trifluoromethylphenyl)amino]methyl}(hydroxyphosphinyl))methyl]pent anedioic acid;
2-[({[(Thioformanilido)amino]benzyl}(hydroxyphosphinyl))methyl]pentanedioic acid;
2-[({[1,3-Dioxo-2,3-dihydro-1H-2-isoindolyl]methyl}hydroxyphosphinyl)methyl]pentanedioic acid.

Preferred PSMA ligands of this section can be selected from the group consisting of:
2-[({[Benzylamino]benzyl}(hydroxyphosphinyl))methyl]pentanedioic acid (1);
2-[({[Carboxyamino]benzyl}(hydroxyphosphinyl))methyl]pentanedioic acid (2);
2-[({[Benzylamino]methyl}(hydroxyphosphinyl))methyl]pentanedioic acid (15);
2-[({[Acetylamino]methyl}(hydroxyphosphinyl))methyl]pentanedioic acid (4);
2-[({[Diphenylamino]methyl}(hydroxyphosphinyl))methyl]pentanedioic acid (5);
2-[({[1,3-Dioxo-2,3-dihydro-1H-2-isoindolyl]methyl}hydroxyphosphinyl)methyl]pen tanedioic acid (6);
2-[({[Phenylamino]methyl}(hydroxyphosphinyl))methyl]pentanedioic acid (7);
2-({[(Phenylcarboxamido)methyl](hydroxyphosphinyl)}methyl)pentanedioic acid (8);
2-({[(Phenylsulfonamido)methyl](hydroxyphosphinyl)}methyl)pentanedioic acid (9);
2-[({[(4-Fluorophenyl)amino]methyl}(hydroxyphosphinyl))methyl]pentanedioic acid (10);
2-[({[(4-M ethoxyphenyl)amino]methyl)}(hydroxyphosphinyl))methyl]pentanedioic acid (11);
2-[({[(4-Methylphenyl)amino]methyl}(hydroxyphosphinyl))methyl]pentanedioic acid (12);
2-[({[(4-Tert-butylphenyl)amino]methyl}(hydroxyphosphinyl))methyl]pentanedioic acid (13); and
2-[({[(Thioformanilido)amino]benzyl}(hydroxyphosphinyl))methyl]pentanedioic acid (14).

### PMSA Ligands (D.5)

In various embodiments, the PSMA ligands represented by variable A in Structural Formulas A1, A2, A3, A11, A12, and A14 can be those described in U.S. Pat. No. 6,384,022, or the patents and applications to which it claims priority, U.S. Pat. Nos. 6,046,180; 6,025,344; 5,795,877; 5,863,536; and 5,672,592. The entire teachings of each of these documents are incorporated herein by reference. The variables and terms in this section can be as described herein, more typically as described in this section, or in preferred embodiments can be as described in the documents incorporated by reference in this paragraph.

In various embodiments, the PSMA ligands represented by variable A in Structural Formulas A1, A2, A3, A11, A12, and A14 can be represented by the following Structural Formula:

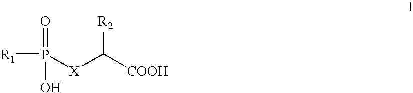

wherein:

X is CR₃R₄, O or NR₅;

R₁ and R₅ are independently selected from the group consisting of hydrogen, C₁-C₉ straight or branched chain alkyl, C₂-C₉ straight or branched chain alkenyl, C₃-C₈ cycloalkyl, C₅-C₇ cycloalkenyl and Ar, wherein said R₁ and R₅ are independently unsubstituted or substituted with one or more substituent(s) independently selected from the group consisting of carboxy, C₃-C₈ cycloalkyl, C₅-C₇ cycloalkenyl, halo, hydroxy, nitro, trifluoromethyl, C₁-C₆ straight or branched chain alkyl, C₂-C₆ straight or branched chain alkenyl, C₁-C₉ alkoxy, C₂-C₉ alkenyloxy, phenoxy, benzyloxy, amino, and Ar;

R₃ and R₄ are independently selected from the group consisting of hydrogen, C₁-C₆ straight or branched chain alkyl, C₂-C₆ straight or branched chain alkenyl, C₃-C₈ cycloalkyl, C₅-C₇ cycloalkenyl, Ar, and halo;

R₂ is selected from the group consisting of hydrogen, C₁-C₉ straight or branched chain alkyl, C₂-C₉ straight or branched chain alkenyl, C₃-C₈ cycloalkyl, C₅-C₇ cycloalkenyl and Ar, wherein said R₂ is unsubstituted or substituted with one or more substituent(s) independently selected from the group consisting of carboxy, C₃-C₈ cycloalkyl, C₅-C₇ cycloalkenyl, halo, hydroxy, nitro, trifluoromethyl, C₁-C₆ straight or branched chain alkyl, C₂-C₆ straight or branched chain alkenyl, C₁-C₆ alkoxy, C₂-C₆ alkenyloxy, phenoxy, benzyloxy, amino, and Ar;

Ar is selected from the group consisting of 1-naphthyl, 2-naphthyl, 2-indolyl, 3-indolyl, 4-indolyl, 2-furyl, 3-furyl, tetrahydrofuranyl, tetrahydropyranyl, 2-thienyl, 3-thienyl, 2-pyridyl, 3-pyridyl, 4-pyridyl, benzyl and phenyl, wherein said Ar is unsubstituted or substituted with one or more substituent(s) independently selected from the group consisting of carboxy, halo, hydroxy, nitro, trifluoromethyl, C₁-C₆ straight or branched chain alkyl, C₂-C₆ straight or branched chain alkenyl, C₁-C₆ alkoxy, C₂-C₆ alkenyloxy, phenoxy, benzyloxy, and amino.

PSMA ligands of this section can be represented by formula II

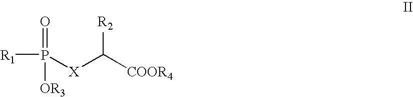

wherein:

X is CR₅R₆,NR₇ or O;

R₁ and R₇ are independently selected from the group consisting of hydrogen, C₁-C₉ straight or branched chain alkyl, C₂-C₉ straight or branched chain alkenyl, C₃-C₈ cycloalkyl, C₅-C₇ cycloalkenyl and Ar₁, wherein said R₁ and R₇ are independently unsubstituted or substituted with one or more substituent(s) independently selected from the group consisting of carboxy, C₃-C₈ cycloalkyl, C₅-C₇ cycloalkenyl, halo, hydroxy, nitro, trifluoromethyl, C₁-C₆ straight or branched chain alkyl, C₂-C₆ straight or branched chain alkenyl, C₁-C₉ alkoxy, C₂-C₉ alkenyloxy, phenoxy, benzyloxy, amino, and Ar₂;

R₂ is selected from the group consisting of hydrogen, C₁-C₉ straight or branched chain alkyl, C₂-C₉ straight or branched chain alkenyl, C₃-C₈ cycloalkyl, C₅-C₇ cycloalkenyl and Ar₁, wherein said R₂ is unsubstituted or substituted with one or more substituent(s) independently selected from the group consisting of carboxy, C₃-C₈ cycloalkyl, C₅-C₇ cycloalkenyl, halo, hydroxy, nitro, trifluoromethyl, C₁-C₆ straight or branched chain alkyl, C₂-C₆ straight or branched chain alkenyl, C₁-C₆ alkoxy, C₂-C₆ alkenyloxy, phenoxy, benzyloxy, amino, and

Ar₂;

R₃ and R₄ are independently selected from the group consisting of hydrogen, carboxy, C₁-C₉ straight or branched chain alkyl, C₂-C₉ straight or branched chain alkenyl, C₃-C₈ cycloalkyl, C₅-C₇ cycloalkenyl, and Ar₁, provided that both R₃ and R₄ are not hydrogen; wherein said R₃ and R₄ are independently unsubstituted or substituted with one or more substituent(s) independently selected from the group consisting of carboxy, C₃-C₈ cycloalkyl, C₅-C₇ cycloalkenyl, halo, hydroxy, nitro, trifluoromethyl, C₁-C₆ straight or branched chain alkyl, C₂-C₆ straight or branched chain alkenyl, C₁-C₆ alkoxy, C₂-C₆ alkenyloxy, phenoxy, benzyloxy, amino, and Ar₂;

R₅ and R₆ are independently selected from the group consisting of hydrogen, C₁-C₆ straight or branched chain alkyl, C₂-C₆ straight or branched chain alkenyl, C₃-C₈ cycloalkyl, C₅-C₇ cycloalkenyl, Ar₁, and halo;

Ar₁ and Ar₂ are independently selected from the group consisting of 1-naphthyl, 2-naphthyl, 2-indolyl, 3-indolyl, 4-indolyl, 2-furyl, 3-furyl, tetrahydrofuranyl, tetrahydropyranyl, 2-thienyl, 3-thienyl, 2-pyridyl, 3-pyridyl, 4-pyridyl, benzyl and phenyl, wherein said Ar₁ and Ar₂ are independently unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₆ alkoxy, C₂-C₆ alkenyloxy, phenoxy, benzyloxy, and amino.

In various embodiments, the PSMA ligands represented by variable A in Structural Formulas A1, A2, A3, A11, A12, and A14 can be selected from the group consisting of:
2-(phosphonomethyl)pentanedioic acid;
2-(phosphonomethyl)succinic acid;
2-[[(2-carboxyethyl)hydroxyphosphinyl]methyl]pentanedioic acid;
2-[[methylhydroxyphosphinyl]methyl]pentanedioic acid;
2-[[ethylhydroxyphosphinyl]methyl]pentanedioic acid;
2-[[propylhydroxyphosphinyl]methyl]pentanedioic acid;
2-[[butylhydroxyphosphinyl]methyl]pentanedioic acid;
2-[[cyclohexylhydroxyphosphinyl]methyl]pentanedioic acid;
2-[[(cyclohexyl)methylhydroxyphosphinyl]methyl]pentanedioic acid;
2-[[phenylhydroxyphosphinyl]methyl]pentanedioic acid;
2-[[(benzylhydroxyphosphinyl)methyl]pentanedioic acid;
2-[[(phenylmethyl)hydroxyphosphinyl]methyl]pentanedioic acid;
2-[[(phenylethyl)hydroxyphosphinyl]methyl]pentanedioic acid;
2-[[(phenylpropyl)hydroxyphosphinyl]methyl]pentanedioic acid;
2-[[(phenylbutyl)hydroxyphosphinyl]methyl]pentanedioic acid;
2-[[(4-methylbenzyl)hydroxyphosphinyl]methyl]pentanedioic acid;
2-[[(4-fluorobenzyl)hydroxyphosphinyl]methyl]pentanedioic acid;
2-[[(2-fluorobenzyl)hydroxyphosphinyl]methyl]pentanedioic acid;
2-[[(pentafluorobenzyl)hydroxyphosphinyl]methyl]pentanedioic acid;
2-[[(methoxybenzyl)hydroxyphosphinyl]methyl]pentanedioic acid;
2-[[(2,3,4-trimethoxyphenyl)hydroxyphosphinyl]methyl]pentanedioic acid;
2-[[(phenylprop-2-enyl)hydroxyphosphinyl]methyl]pentanedioic acid;
2-[[(2-fluorobenzyl)hydroxyphosphinyl]methyl]pentanedioic acid;
2-[[((hydroxy)phenylmethyl)hydroxyphosphinyl]methyl]pentanedioic acid;
2-[[(3-methylbenzyl)hydroxyphosphinyl]methyl]pentanedioic acid;
2-[[(4-fluorophenyl)hydroxyphosphinyl]methyl]pentanedioic acid;
2-[[(3-trifluoromethylbenzyl)hydroxyphosphinyl]methyl]pentanedioic acid.

In another preferred embodiment of formula I, R₂ is C₃-C₉ alkyl; R₁ is 2-indolyl, 3-indolyl, 4-indolyl, 2-furyl, 3-furyl, tetrahydrofuranyl, tetrahydropyranyl, 2-thienyl, 3-thienyl, 2-pyridyl, 3-pyridyl, 4-pyridyl or C₁-C₄ straight or branched chain alkyl substituted with 2-indolyl, 3-indolyl, 4-indolyl, 2-furyl, 3-furyl, tetrahydrofuranyl, 2-thienyl, 3-thienyl, 2-pyridyl, 3-pyridyl or 4-pyridyl; or R₁ is 1-naphthyl, 2-naphthyl, or C₁-C₄ straight or branched chain alkyl substituted with 1-naphthyl or 2-naphthyl.

In various embodiments, the PSMA ligands represented by variable A in Structural Formulas A1, A2, A3, A11, A12, and A14 can be selected from the group consisting of:
2-[(methylhydroxyphosphinyl)methyl]hexanedioic acid;
2-[(benzylhydroxyphosphinyl)methyl]hexanedioic acid;
2-[(methylhydroxyphosphinyl)methyl]heptanedioic acid;
2-[(benzylhydroxyphosphinyl)methyl]heptanedioic acid;
2-[(methylhydroxyphosphinyl)methyl]octanedioic acid;
2-[(benzylhydroxyphosphinyl)methyl]octanedioic acid;
2-[(methylhydroxyphosphinyl)methyl]nonanedioic acid;
2-[(benzylhydroxyphosphinyl)methyl]nonanedioic acid;
2-[(methylhydroxyphosphinyl)methyl]decanedioic acid;
2-[(benzylhydroxyphosphinyl)methyl]decanedioic acid;
2-[[(2-pyridyl)methylhydroxyphosphinyl]methyl]pentanedioic acid;
2-[[(3-pyridyl)methylhydroxyphosphinyl]methyl]pentanedioic acid;
2-[[(4-pyridyl)methylhydroxyphosphinyl]methyl]pentanedioic acid;
2-[[(3-pyridyl)ethylhydroxyphosphinyl]methyl]pentanedioic acid;
2-[[(3-pyridyl)propylhydroxyphosphinyl]methyl]pentanedioic acid;
2-[[(tetrahydrofuranyl)methylhydroxyphosphinyl]methyl]pentanedioic acid;
2-[[(tetrahydrofuranyl)ethylhydroxyphosphinyl]methyl]pentanedioic acid;
2-[[(tetrahydrofuranyl)propylhydroxyphosphinyl]methyl]pentanedioic acid;
2-[[(2-tetrahydropyranyl)hydroxyphosphinyl]methyl]pentanedioic acid;
2-[[(3-tetrahydropyranyl)hydroxyphosphinyl]methyl]pentanedioic acid;
2-[[(4-tetrahydropyranyl)hydroxyphosphinyl]methyl]pentanedioic acid;
2-[[(2-indolyl)methylhydroxyphosphinyl]methyl]pentanedioic acid;
2-[[(3-indolyl)methylhydroxyphosphinyl]methyl]pentanedioic acid;
2-[[(4-indolyl)methylhydroxyphosphinyl]methyl]pentanedioic acid;
2-[[(3-indolyl)ethylhydroxyphosphinyl]methyl]pentanedioic acid;
2-[[(3-indolyl)propylhydroxyphosphinyl]methyl]pentanedioic acid;
2-[[(2-thienyl)methylhydroxyphosphinyl]methyl]pentanedioic acid;
2-[[(3-thienyl)methylhydroxyphosphinyl]methyl]pentanedioic acid;
2-[[(4-thienyl)methylhydroxyphosphinyl]methyl]pentanedioic acid;
2-[[(3-thienyl)ethylhydroxyphosphinyl]methyl]pentanedioic acid;
2-[[(3-thienyl)propylhydroxyphosphinyl]methyl]pentanedioic acid;
2-[[(2-pyridyl)hydroxyphosphinyl]methyl]pentanedioic acid;
2-[[(3-pyridyl)hydroxyphosphinyl]methyl]pentanedioic acid;
2-[[(4-pyridyl)hydroxyphosphinyl]methyl]pentanedioic acid;
2-[[(tetrahydrofuranyl)hydroxyphosphinyl]methyl]pentanedioic acid;
2-[[(2-indolyl)hydroxyphosphinyl]methyl]pentanedioic acid;
2-[[(3-indolyl)hydroxyphosphinyl]methyl]pentanedioic acid;
2-[[(4-indolyl)hydroxyphosphinyl]methyl]pentanedioic acid;
2-[[2-thienyl)hydroxyphosphinyl]methyl]pentanedioic acid;
2-[[(3-thienyl)hydroxyphosphinyl]methyl]pentanedioic acid;
2-[[(4-thienyl)hydroxyphosphinyl]methyl]pentanedioic acid;
2-[[(1-naphthyl)hydroxyphosphinyl]methyl]pentanedioic acid;
2-[[(2-naphthyl)hydroxyphosphinyl]methyl]pentanedioic acid;
2-[[(1-naphthyl)methylhydroxyphosphinyl]methyl]pentanedioic acid;
2-[[(2-naphthyl)methylhydroxyphosphinyl]methyl]pentanedioic acid;
2-[[(1-naphthyl)ethylhydroxyphosphinyl]methyl]pentanedioic acid;
2-[[(2-naphthyl)ethylhydroxyphosphinyl]methyl]pentanedioic acid;
2-[[(1-naphthyl)propylhydroxyphosphinyl]methyl]pentanedioic acid;
2-[[(2-naphthyl)propylhydroxyphosphinyl]methyl]pentanedioic acid;
2-[[(1-naphthyl)butylhydroxyphosphinyl]methyl]pentanedioic acid;
2-[[(2-naphthyl)butylhydroxyphosphinyl]methyl]pentanedioic acid.

In another preferred embodiment of formula I, X is CH₂ and R₂ is selected from the group consisting of hydrogen, C₁-C₉ straight or branched chain alkyl, C₂-C₉ straight or branched chain alkenyl, C₃-C₈ cycloalkyl, C₅-C₇ cycloalkenyl, benzyl and phenyl, wherein said R₂ is unsubstituted or substituted with one or more substituent(s) independently selected from the group consisting of C₃-C₈ cycloalkyl, C₅-C₇ cycloalkenyl, C₁-C₆ straight or branched chain alkyl, C₂-C₆ straight or branched chain alkenyl, C₁-C₄ alkoxy, and phenyl.

More preferably, R₁ is hydrogen, C₁-C₄ straight or branched chain alkyl, C₂-C₄ straight or branched chain alkenyl, C₃-C₈ cycloalkyl, C₅-C₇ cycloalkenyl, benzyl or phenyl, wherein said R₁ is unsubstituted or substituted with one or more substituent(s) independently selected from the group consisting of carboxy, C₃-C₈ cycloalkyl, C₅-C₇ cycloalkenyl, halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, amino, benzyl, and phenyl.

In various embodiments, the PSMA ligands represented by variable A in Structural Formulas A1, A2, A3, A11, A12, and A14 can be selected from the group consisting of:
3-(methylhydroxyphosphinyl)-2-phenylpropanoic acid;
3-(ethylhydroxyphosphinyl)-2-phenylpropanoic acid;
3-(propylhydroxyphosphinyl)-2-phenylpropanoic acid;
3-(butylhydroxyphosphinyl)-2-phenylpropanoic acid;
3-(cyclohexylhydroxyphosphinyl)-2-phenylpropanoic acid;
3-((cyclohexyl)methylhydroxyphosphinyl)-2-phenylpropanoic acid;
3-(phenylhydroxyphosphinyl)-2-phenylpropanoic acid;
3-(benzylhydroxyphosphinyl)-2-phenylpropanoic acid;
3-(phenylethylhydroxyphosphinyl)-2-phenylpropanoic acid;
3-(phenylpropylhydroxyphosphinyl)-2-phenylpropanoic acid;
3-(phenylbutylhydroxyphosphinyl)-2-phenylpropanoic acid;
3-((2,3,4-trimethoxyphenyl)-3-hydroxyphosphinyl)-2-phenylpropanoic acid;
3-(phenylprop-2-enylhydroxyphosphinyl)-2-phenylpropanoic acid;
3-(benzylhydroxyphosphinyl)-2-ethylpropanoic acid;
3-(benzylhydroxyphosphinyl)-2-propylpropanoic acid;
3-(benzylhydroxyphosphinyl)-2-butylpropanoic acid;
3-(benzylhydroxyphosphinyl)-2-cyclohexylpropanoic acid;
3-(benzylhydroxyphosphinyl)-2-(cyclohexyl)methylpropanoic acid;
3-(benzylhydroxyphosphinyl)-2-phenylpropanoic acid;
3-(benzylhydroxyphosphinyl)-2-benzylpropanoic acid;
3-(benzylhydroxyphosphinyl)-2-phenylethylpropanaic acid;
3-(benzylhydroxyphosphinyl)-2-phenylpropylpropanoic acid;
3-(benzylhydroxyphosphinyl)-2-phenylbutylpropanoic acid;
3-(benzylhydroxyphosphinyl)-2-(2,3,4-trimethoxyphenyl)propanoic acid; and
3-(benzylhydroxyphosphinyl)-2-phenylprop-2-enylpropanoic acid.

In a further embodiment of formula I, at least one of R₁ and R₂ is 2-indolyl, 3-indolyl, 4-indolyl, 2-furyl, 3-furyl, tetrahydrofuranyl, tetrahydropyranyl, 2-thienyl, 3-thienyl, 2-pyridyl, 3-pyridyl, 4-pyridyl, or C₁-C₄ straight or branched chain alkyl substituted with 2-indolyl 3-indolyl, 4-indolyl, 2-furyl, 3-furyl, tetrahydrofuranyl, 2-thienyl, 3-thienyl, 2-pyridyl, 3-pyridyl or 4-pyridyl; or R₁ is 1-naphthyl, 2-naphthyl, or C₁-C₄ straight or branched chain alkyl substituted with 1-naphthyl or 2-naphthyl.

In various embodiments, the PSMA ligands represented by variable A in Structural Formulas A1, A2, A3, A11, A12, and A14 can be selected from the group consisting of:
3-[(2-pyridyl)methylhydroxyphosphinyl]-2-phenylpropanoic acid;
3-[(3-pyridyl)methylhydroxyphosphinyl]-2-phenylpropanoic acid;
3-[(4-pyridyl)methylhydroxyphosphinyl]-2-phenylpropanoic acid;
3-[(3-pyridyl)ethylhydroxyphosphinyl]-2-phenylpropanoic acid;
3-[(3-pyridyl)propylhydroxyphosphinyl]-2-phenylpropanoic acid;
3-[(tetrahydrofuranyl)methylhydroxyphosphinyl]-2-phenylpropanoic acid;
3-[(tetrahydrofuranyl)ethylhydroxyphosphinyl]-2-phenylpropanoic acid;
3-[(tetrahydrofuranyl)propylhydroxyphosphinyl]-2-phenylpropanoic acid;
3-[(2-indolyl)methylhydroxyphosphinyl]-2-phenylpropanoic acid;
3-[(3-indolyl)methylhydroxyphosphinyl]-2-phenylpropanoic acid;
3-[(4-indolyl)methylhydroxyphosphinyl]-2-phenylpropanoic acid;
3-[(3-indolyl)ethylhydroxyphosphinyl]-2-phenylpropanoic acid;
3-[(3-indolyl)propylhydroxyphosphinyl]-2-phenylpropanoic acid;
3-[(2-thienyl)methylhydroxyphosphinyl]-2-phenylpropanoic acid;
3-[(3-thienyl)methylhydroxyphosphinyl]-2-phenylpropanoic acid;
3-[(4-thienyl)methylhydroxyphosphinyl]-2-phenylpropanoic acid;
3-[(3-thienyl)ethylhydroxyphosphinyl]-2-phenylpropanoic acid;
3-[(3-thienyl)propylhydroxyphosphinyl]-2-phenylpropanoic acid;
3-(benzylhydroxyphosphinyl)-2-(2-pyridyl)methylpropanoic acid;
3-(benzylhydroxyphosphinyl)-2-(3-pyridyl)methylpropanoic acid;
3-(benzylhydroxyphosphinyl)-2-(4-pyridyl)methylpropanoic acid;
3-(benzylhydroxyphosphinyl)-2-(3-pyridyl)ethylpropanoic acid;
3-(benzylhydroxyphosphinyl)-2-(3-pyridyl)propylpropanoic acid;
3-(benzylhydroxyphosphinyl)-2-(tetrahydrofuranyl)methylpropanoic acid;
3-(benzylhydroxyphosphinyl)-2-(tetrahydrofuranyl)ethylpropanoic acid;
3-(benzylhydroxyphosphinyl)-2-(tetrahydrofuranyl)propylpropanoic acid;
3-(benzylhydroxyphosphinyl)-2-(2-indolyl)methylpropanoic acid;
3-(benzylhydroxyphosphinyl)-2-(3-indolyl)methylpropanoic acid;
3-(benzylhydroxyphosphinyl)-2-(4-indolyl)methylpropanoic acid;
3-(benzylhydroxyphosphinyl)-2-(3-indolyl)ethylpropanoic acid;
3-(benzylhydroxyphosphinyl)-2-(3-indolyl)propylpropanoic acid;
3-(benzylhydroxyphosphinyl)-2-(2-thienyl)methylpropanoic acid;
3-(benzylhydroxyphosphinyl)-2-(3-thienyl)methylpropanoic acid;
3-(benzylhydroxyphosphinyl)-2-(4-thienyl)methylpropanoic acid;
3-(benzylhydroxyphosphinyl)-2-(3-thienyl)ethylpropanoic acid;
3-(benzylhydroxyphosphinyl)-2-(3-thienyl)propylpropanoic acid;
3-((1-naphthyl)hydroxyphosphinyl)-2-phenylpropanoic acid;
3-((2-naphthyl)hydroxyphosphinyl)-2-phenylpropanoic acid;
3-((1-naphthyl)methylhydroxyphosphinyl)-2-phenylpropanoic acid;
3-((2-naphthyl)methylhydroxyphosphinyl)-2-phenylpropanoic acid;
3-((1-naphthyl)ethylhydroxyphosphinyl)-2-phenylpropanoic acid;
3-((2-naphthyl)ethylhydroxyphosphinyl)-2-phenylpropanoic acid;
3-((1-naphthyl)propylhydroxyphosphinyl)-2-phenylpropanoic acid;
3-((2-naphthyl)propylhydroxyphosphinyl)-2-phenylpropanoic acid;
3-((1-naphthyl)butylhydroxyphosphinyl)-2-phenylpropanoic acid; nd
3-((2-naphthyl)butylhydroxyphosphinyl)-2-phenylpropanoic acid.

In various embodiments, the PSMA ligands represented by variable A in Structural Formulas A1, A2, A3, A11, A12, and A14 can be selected from the group consisting of:
2-[[methylhydroxyphosphinyl]oxy]pentanedioic acid;
2-[[ethylhydroxyphosphinyl]oxy]pentanedioic acid;
2-[[propylhydroxyphosphinyl]oxy]pentanedioic acid;
2-[[butylhydroxyphosphinyl]oxy]pentanedioic acid;
2-[[cyclohexylhydroxyphosphinyl]oxy]pentanedioic acid;
2-[[(cyclohexyl)methylhydroxyphosphinyl]oxy]pentanedioic acid;
2-[[phenylhydroxyphosphinyl]oxy]pentanedioic acid;
2-[[benzylhydroxyphosphinyl]oxy]pentanedioic acid;
2-[[phenylethylhydroxyphosphinyl]oxy]pentanedioic acid;
2-[[phenylpropylhydroxyphosphinyl]oxy]pentanedioic acid;
2-[[phenylbutylhydroxyphosphinyl]oxy]pentanedioic acid;
2-[[(4-methylbenzyl)hydroxyphosphinyl]oxy]pentanedioic acid;
2-[[(4-fluorobenzyl)hydroxyphosphinyl]oxy]pentanedioic acid;
2-[[(2-fluorobenzyl)hydroxyphosphinyl]oxy]pentanedioic acid;
2-[[(pentafluorobenzyl)hydroxyphosphinyl]oxy]pentanedioic acid;
2-[[(methoxybenzyl)hydroxyphosphinyl]oxy]pentanedioic acid;
2-[[(2,3,4-trimethoxyphenyl)hydroxyphosphinyl]oxy]pentanedioic acid;
2-[[(1-naphthyl)hydroxyphosphinyl]oxy]pentanedioic acid;
2-[[(2-naphthyl)hydroxyphosphinyl]oxy]pentanedioic acid;
2-[[(1-naphthyl)methylhydroxyphosphinyl]oxy]pentanedioic acid;
2-[[(2-naphthyl)methylhydroxyphosphinyl]oxy]pentanedioic acid;
2-[[(1-naphthyl)ethylhydroxyphosphinyl]oxy]pentanedioic acid;
2-[[(2-naphthyl)ethylhydroxyphosphinyl]oxy]pentanedioic acid;
2-[[(1-naphthyl)propylhydroxyphosphinyl]oxy]pentanedioic acid;
2-[[(2-naphthyl)propylhydroxyphosphinyl]oxy]pentanedioic acid;
2-[[(1-naphthyl)butylhydroxyphosphinyl]oxy]pentanedioic acid;
2-[[(2-naphthyl)butylhydroxyphosphinyl]oxy]pentanedioic acid;
2-[[(phenylprop-2-enyl)hydroxyphosphinyl]oxy]pentanedioic acid;
2-[[benzylhydroxyphosphinyl]oxy]pentanedioic acid;
2-[[((hydroxy)phenylmethyl)hydroxyphosphinyl]oxy]pentanedioic acid;
2-[[(3-methylbenzyl)hydroxyphosphinyl]oxy]pentanedioic acid;
2-[[(4-fluorophenyl)hydroxyphosphinyl]oxy]pentanedioic acid;
2-[[(2-fluorobenzyl)hydroxyphosphinyl]oxy]pentanedioic acid;
2-[(phosphono)oxy]pentanedioic acid;
2-[[(3-trifluoromethylbenzyl)hydroxyphosphinyl]oxy]pentanedioic acid;
2-[[methylhydroxyphosphinyl]oxy]-2-phenylethanoic acid;
2-[[ethylhydroxyphosphinyl]oxy]-2-phenylethanoic acid;
2-[[propylhydroxyphosphinyl]oxy]-2-phenylethanoic acid;
2-[[butylhydroxyphosphinyl]oxy]-2-phenylethanoic acid;
2-[[cyclohexylhydroxyphosphinyl]oxy]-2-phenylethanoic acid;
2-[[(cyclohexyl)methylhydroxyphosphinyl]oxy]-2-phenylethanoic acid;
2-[[phenylhydroxyphosphinyl]oxy]-2-phenylethanoic acid;
2-[[benzylhydroxyphosphinyl]oxy]-2-phenylethanoic acid;
2-[[phenylethylhydroxyphosphinyl]oxy]-2-phenylethanoic acid;
2-[[phenylpropylhydroxyphosphinyl]oxy]-2-phenylethanoic acid;
2-[[phenylbutylhydroxyphosphinyl]oxy]-2-phenylethanoic acid;
2-[[(2,3,4-trimethoxyphenyl)-3-hydroxyphosphinyl]oxy]-2-phenylethanoic acid;
2-[[(1-naphthyl)hydroxyphosphinyl]oxy]-2-phenylethanoic acid;
2-[[(2-naphthyl)hydroxyphosphinyl]oxy]-2-phenylethanoic acid;
2-[[(1-naphthyl)methylhydroxyphosphinyl]oxy]-2-phenylethanoic acid;
2-[[(2-naphthyl)methylhydroxyphosphinyl]oxy]-2-phenylethanoic acid;
2-[[(2-naphthyl)ethylhydroxyphosphinyl]oxy]-2-phenylethanoic acid;
2-[[(1-naphthyl)ethylhydroxyphosphinyl]oxy]-2-phenylethanoic acid;
2-[[(2-naphthyl)propylhydroxyphosphinyl]oxy]-2-phenylethanoic acid;
2-[[(2-naphthyl)propylhydroxyphosphinyl]oxy]-2-phenylethanoic acid;
2-[[(1-naphthyl)butylhydroxyphosphinyl]oxy]-2-phenylethanoic acid;
2-[[(2-naphthyl)propylhydroxyphosphinyl]oxy]-2-phenylethanoic acid;
2-[[phenylprop-2-enylhydroxyphosphinyl]oxy]-2-phenylethanoic acid;
2-[(methylhydroxyphosphinyl)oxy]hexanedioic acid;
2-[(benzylhydroxyphosphinyl)oxy]hexanedioic acid;
2-[(methylhydroxyphosphinyl)oxy]heptanedioic acid;
2-[(benzylhydroxyphosphinyl)oxy]heptanedioic acid;
2-[(methylhydroxyphosphinyl)oxy]octanedioic acid;
2-[(benzylhydroxyphosphinyl)oxy]octanedioic acid;
2-[(methylhydroxyphosphinyl)oxy]nonanedioic acid;
2-[(benzylhydroxyphosphinyl)oxy]nonanedioic acid;
2-[(methylhydroxyphosphinyl)oxy]decanedioic acid;
2-[(benzylhydroxyphosphinyl)oxy]decanedioic acid;
2-[[benzylhydroxyphosphinyl]oxy]-2-methylethanoic acid;
2-[[benzylhydroxyphosphinyl]oxy]-2-ethylethanoic acid;
2-[[benzylhydroxyphosphinyl]oxy]-2-propylethanoic acid;
2-[[benzylhydroxyphosphinyl]oxy]-2-butylethanoic acid;
2-[[benzylhydroxyphosphinyl]oxy]-2-cyclohexylethanoic acid;
2-[[benzylhydroxyphosphinyl]oxy]-2-(cyclohexyl)methylethanoic acid;
2-[[benzylhydroxyphosphinyl]oxy]-2-phenylethanoic acid;
2-[[benzylhydroxyphosphinyl]oxy]-2-benzylethanoic acid;
2-[[benzylhydroxyphosphinyl]oxy]-2-phenylethylethanoic acid;
2-[[benzylhydroxyphosphinyl]oxy]-2-phenylpropylethanoic acid;
2-[[benzylhydroxyphosphinyl]oxy]-2-phenylbutylethanoic acid;
2-[[benzylhydroxyphosphinyl]oxy]-2-(2,3,4-trimethoxyphenyl)ethanoic acid;
2-[[benzylhydroxyphosphinyl]oxy]-2-(1-naphthyl)ethanoic acid;
2-[[benzylhydroxyphosphinyl]oxy]-2-(2-naphthyl)ethanoic acid;
2-[[benzylhydroxyphosphinyl]oxy]-2-(1-naphthyl)methylethanoic acid;
2-[[benzylhydroxyphosphinyl]oxy]-2-(2-naphthyl)methylethanoic acid;
2-[[benzylhydroxyphosphinyl]oxy]-2-(1-naphthyl)ethylethanoic acid;
2-[[benzylhydroxyphosphinyl]oxy]-2-(2-naphthyl)ethylethanoic acid;
2-[[benzylhydroxyphosphinyl]oxy]-2-(1-naphthyl)propylethanoic acid;
2-[[benzylhydroxyphosphinyl]oxy]-2-(2-naphthyl)propylethanoic acid;
2-[[benzylhydroxyphosphinyl]oxy]-2-(1-naphthyl)butylethanoic acid;
2-[[benzylhydroxyphosphinyl]oxy]-2-(2-naphthyl)butylethanoic acid;
2-[[benzylhydroxyphosphinyl]oxy]-2-phenylprop-2-enylethanoic acid;
2-[[(2-pyridyl)methylhydroxyphosphinyl]oxy]pentanedioic acid;
2-[[(3-pyridyl)methylhydroxyphosphinyl]oxy]pentanedioic acid;
2-[[(4-pyridyl)methylhydroxyphosphinyl]oxy]pentanedioic acid;
2-[[(3-pyridyl)ethylhydroxyphosphinyl]oxy]pentanedioic acid;
2-[[(3-pyridyl)propylhydroxyphosphinyl]oxy]pentanedioic acid;
2-[[(tetrahydrofuranyl)methylhydroxyphosphinyl]oxy]pentanedioic acid;
2-[[(tetrahydrofuranyl)ethylhydroxyphosphinyl]oxy]pentanedioic acid;
2-[[(tetrahydrofuranyl)propylhydroxyphosphinyl]oxy]pentanedioic acid;
2-[[(2-indolyl)methylhydroxyphosphinyl]oxy]pentanedioic acid;
2-[[(3-indolyl)methylhydroxyphosphinyl]oxy]pentanedioic acid;
2-[[(4-indolyl)methylhydroxyphosphinyl]oxy]pentanedioic acid;
2-[[(3-indolyl)ethylhydroxyphosphinyl]oxy]pentanedioic acid;
2-[[(3-indolyl)propylhydroxyphosphinyl]oxy]pentanedioic acid;
2-[[(2-thienyl)methylhydroxyphosphinyl]oxy]pentanedioic acid;
2-[[(3-thienyl)methylhydroxyphosphinyl]oxy]pentanedioic acid;
2-[[(4-thienyl)methylhydroxyphosphinyl]oxy]pentanedioic acid;
2-[[(3-thienyl)ethylhydroxyphosphinyl]oxy]pentanedioic acid; and
2-[[(3-thienyl)propylhydroxyphosphinyl]oxy]pentanedioic acid.

In another preferred embodiment of the PSMA ligands of this section, R₂ is selected from the group consisting of hydrogen, C₁-C₉ straight or branched chain alkyl, C₂-C₉ straight or branched chain alkenyl, C₃-C₈ cycloalkyl, C₅-C₇ cycloalkenyl, benzyl and phenyl, wherein said R₂ is unsubstituted or substituted with one or more substituent(s) independently selected from the group consisting of C₃-C₈ cycloalkyl, C₅-C₇ cycloalkenyl, C₁-C₆ straight or branched chain alkyl, C₂-C₆ straight or branched chain alkenyl, C₁-C₄ alkoxy, and phenyl.

In various embodiments, the PSMA ligands represented by variable A in Structural Formulas A1, A2, A3, A11, A12, and A14 can be selected from the group consisting of:
2-[[(2-pyridyl)methylhydroxyphosphinyl]oxy]-2-phenylethanoic acid;
2-[[(3-pyridyl)methylhydroxyphosphinyl]oxy]-2-phenylethanoic acid;
2-[[(4-pyridyl)methylhydroxyphosphinyl]oxy]-2-phenylethanoic acid;
2-[[(3-pyridyl)ethylhydroxyphosphinyl]oxy]-2-phenylethanoic acid;
2-[[(3-pyridyl)propylhydroxyphosphinyl]oxy]-2-phenylethanoic acid;
2-[[(tetrahydrofuranyl)methylhydroxyphosphinyl]oxy]-2-phenylethanoic acid;
2-[[(tetrahydrofuranyl)ethylhydroxyphosphinyl]oxy]-2-phenylethanoic acid;
2-[[(tetrahydrofuranyl)propylhydroxyphosphinyl]oxy]-2-phenylethanoic acid;
2-[[(2-indolyl)methylhydroxyphosphinyl]oxy]-2-phenylethanoic acid;
2-[[(3-indolyl)methylhydroxyphosphinyl]oxy]-2-phenylethanoic acid;
2-[[(4-indolyl)methylhydroxyphosphinyl]oxy]-2-phenylethanoic acid;
2-[[(3-indolyl)ethylhydroxyphosphinyl]oxy]-2-phenylethanoic acid;
2-[[(3-indolyl)propylhydroxyphosphinyl]oxy]-2-phenylethanoic acid;
2-[[(2-thienyl)methylhydroxyphosphinyl]oxy]-2-phenylethanoic acid;
2-[[(3-thienyl)methylhydroxyphosphinyl]oxy]-2-phenylethanoic acid;
2-[[(4-thienyl)methylhydroxyphosphinyl]oxy]-2-phenylethanoic acid;
2-[[(3-thienyl)ethylhydroxyphosphinyl]oxy]-2-phenylethanoic acid;
2-[[(3-thienyl)propylhydroxyphosphinyl]oxy]-2-phenylethanoic acid;
2-[[benzylhydroxyphosphinyl]oxy]-2-(2-pyridyl)methylethanoic acid;
2-[[benzylhydroxyphosphinyl]oxy]-2-(3-pyridyl)methylethanoic acid;
2-[[benzylhydroxyphosphinyl]oxy]-2-(4-pyridyl)methylethanoic acid;
2-[[benzylhydroxyphosphinyl]oxy]-2-(3-pyridyl)ethylethanoic acid;
2-[[benzylhydroxyphosphinyl]oxy]-2-(3-pyridyl)propylethanoic acid;
2-[[benzylhydroxyphosphinyl]oxy]-2-(tetrahydrofuranyl)methylethanoic acid;
2-[[benzylhydroxyphosphinyl]oxy]-2-(tetrahydrofuranyl)ethylethanoic acid;
2-[[benzylhydroxyphosphinyl]oxy]-2-(tetrahydrofuranyl)propylethanoic acid;
2-[[benzylhydroxyphosphinyl]oxy]-2-(2-indolyl)methylethanoic acid;
2-[[benzylhydroxyphosphinyl]oxy]-2-(3-indolyl)methylethanoic acid;
2-[[benzylhydroxyphosphinyl]oxy]-2-(4-indolyl)methylethanoic acid;
2-[[benzylhydroxyphosphinyl]oxy]-2-(3-indolyl)ethylethanoic acid;
2-[[benzylhydroxyphosphinyl]oxy]-2-(3-indolyl)propylethanoic acid;
2-[[benzylhydroxyphosphinyl]oxy]-2-(2-thienyl)methylethanoic acid;
2-[[benzylhydroxyphosphinyl]oxy]-2-(3-thienyl)methylethanoic acid;
2-[[benzylhydroxyphosphinyl]oxy]-2-(4-thienyl)methylethanoic acid;
2-[[benzylhydroxyphosphinyl]oxy]-2-(3-thienyl)ethylethanoic acid; and
2-[[benzylhydroxyphosphinyl]oxy]-2-(3-thienyl)propylethanoic acid.

When X is NR₅, R₂ is preferably substituted with carboxy.

In various embodiments, the PSMA ligands represented by variable A in Structural Formulas A1, A2, A3, A11, A12, and A14 can be selected from the group consisting of:
2-[[methylhydroxyphosphinyl]amino]pentanedioic acid;
2-[[ethylhydroxyphosphinyl]amino]pentanedioic acid;
2-[[propylhydroxyphosphinyl]amino]pentanedioic acid;
2-[[butylhydroxyphosphinyl]amino]pentanedioic acid;
2-[[cyclohexylhydroxyphosphinyl]amino]pentanedioic acid;
2-[[(cyclohexyl)methylhydroxyphosphinyl]amino]pentanedioic acid;
2-[[phenylhydroxyphosphinyl]amino]pentanedioic acid;
2-[[benzylhydroxyphosphinyl]amino]pentanedioic acid;
2-[[phenylethylhydroxyphosphinyl]amino]pentanedioic acid;
2-[[phenylpropylhydroxyphosphinyl]amino]pentanedioic acid;
2-[[phenylbutylhydroxyphosphinyl]amino]pentanedioic acid;
2-[[(4-methylbenzyl)hydroxyphosphinyl]amino]pentanedioic acid;
2-[[(4-fluorobenzyl)hydroxyphosphinyl]amino]pentanedioic acid;
2-[[(2-fluorobenzyl)hydroxyphosphinyl]amino]pentanedioic acid;
2-[[(pentafluorobenzyl)hydroxyphosphinyl]amino]pentanedioic acid;
2-[[(methoxybenzyl)hydroxyphosphinyl]amino]pentanedioic acid;
2-[[(2,3,4-trimethoxyphenyl)hydroxyphosphinyl]amino]pentanedioic acid;
2-[[(1-naphthyl)hydroxyphosphinyl]amino]pentanedioic acid;
2-[[(2-naphthyl)hydroxyphosphinyl]amino]pentanedioic acid;
2-[[(1-naphthyl)methylhydroxyphosphinyl]amino]pentanedioic acid;
2-[[(2-naphthyl)methylhydroxyphosphinyl]amino]pentanedioic acid;
2-[[(1-naphthyl)ethylhydroxyphosphinyl]amino]pentanedioic acid;
2-[[(2-naphthyl)ethylhydroxyphosphinyl]amino]pentanedioic acid;
2-[[(1-naphthyl)propylhydroxyphosphinyl]amino]pentanedioic acid;
2-[[(2-naphthyl)propylhydroxyphosphinyl]amino]pentanedioic acid;
2-[[(1-naphthyl)butylhydroxyphosphinyl]amino]pentanedioic acid;
2-[[(2-naphthyl)butylhydroxyphosphinyl]amino]pentanedioic acid;
2-[[(phenylprop-2-enyl)hydroxyphosphinyl]amino]pentanedioic acid;
2-[[benzylhydroxyphosphinyl]amino]pentanedioic acid;
2-[[(2-fluorobenzyl)hydroxyphosphinyl]amino]-2-pentanedioic acid;
2-[[((hydroxy)phenylmethyl)hydroxyphosphinyl]amino]pentanedioic acid;
2-[[(3-methylbenzyl)hydroxyphosphinyl]amino]pentanedioic acid;
2-[[(4-fluorophenyl)hydroxyphosphinyl]amino]pentanedioic acid;
2-[(phosphono)amino]pentanedioic acid;
2-[[(3-trifluoromethylbenzyl)hydroxyphosphinyl]amino]pentanedioic acid;
2-[(methylhydroxyphosphinyl)amino]hexanedioic acid;
2-[(benzylhydroxyphosphinyl)amino]hexanedioic acid;
2-[(methylhydroxyphosphinyl)amino]heptanedioic acid;
2-[(benzylhydroxyphosphinyl)amino]heptanedioic acid;
2-[(methylhydroxyphosphinyl)amino]octanedioic acid;
2-[(benzylhydroxyphosphinyl)amino]octanedioic acid;
2-[(methylhydroxyphosphinyl)amino]nonanedioic acid;
2-[(benzylhydroxyphosphinyl)amino]nonanedioic acid;
2-[(methylhydroxyphosphinyl)amino]decanedioic acid;
2-[(benzylhydroxyphosphinyl)amino]decanedioic acid;
3-[[(2-pyridyl)methylhydroxyphosphinyl]amino]pentanedioic acid;
3-[[(3-pyridyl)methylhydroxyphosphinyl]amino]pentanedioic acid;
3-[[(4-pyridyl)methylhydroxyphosphinyl]amino]pentanedioic acid;
3-[[(3-pyridyl)ethylhydroxyphosphinyl]amino]pentanedioic acid;
3-[[(3-pyridyl)propylhydroxyphosphinyl]amino]pentanedioic acid;
3-[[(tetrahydrofuranyl)methylhydroxyphosphinyl]amino]pentanedioic acid;
3-[[(tetrahydrofuranyl)ethylhydroxyphosphinyl]amino]pentanedioic acid;
3-[[(tetrahydrofuranyl)propylhydroxyphosphinyl]amino]pentanedioic acid;
3-[[(2-indolyl)methylhydroxyphosphinyl]amino]pentanedioic acid;
3-[[(3-indolyl)methylhydroxyphosphinyl]amino]pentanedioic acid;
3-[[(4-indolyl)methylhydroxyphosphinyl]amino]pentanedioic acid;
3-[[(3-indolyl)ethylhydroxyphosphinyl]amino]pentanedioic acid;
3-[[(3-indolyl)propylhydroxyphosphinyl]amino]pentanedioic acid;
3-[[(2-thienyl)methylhydroxyphosphinyl]amino]pentanedioic acid;
3-[[(3-thienyl)methylhydroxyphosphinyl]amino]pentanedioic acid;
3-[[(4-thienyl)methylhydroxyphosphinyl]amino]pentanedioic acid;
3-[[(3-thienyl)ethylhydroxyphosphinyl]amino]pentanedioic acid; and
3-[[(3-thienyl)propylhydroxyphosphinyl]amino]pentanedioic acid.

In another preferred embodiment, R₂ is selected from the group consisting of hydrogen, C₁-C₉ straight or branched chain alkyl, C₂-C₉ straight or branched chain alkenyl, C₃-C₈ cycloalkyl, C₅-C₇ cycloalkenyl, benzyl and phenyl, wherein said R₂ is unsubstituted or substituted with one or more substituent(s) independently selected from the group consisting of C₃-C₈ cycloalkyl, C₅-C₇ cycloalkenyl, C₁-C₆ straight or branched chain alkyl, C₂-C₆ straight or branched chain alkenyl, C₁-C₄ alkoxy, and phenyl.

In various embodiments, the PSMA ligands represented by variable A in Structural Formulas A1, A2, A3, A11, A12, and A14 can be selected from the group consisting of:
2-[[methylhydroxyphosphinyl]amino]-2-phenylethanoic acid;
2-[[ethylhydroxyphosphinyl]amino]-2-phenylethanoic acid;
2-[[propylhydroxyphosphinyl]amino]-2-phenylethanoic acid;
2-[[butylhydroxyphosphinyl]amino]-2-phenylethanoic acid;
2-[[cyclohexylhydroxyphosphinyl]amino]-2-phenylethanoic acid;
2-[[(cyclohexyl)methylhydroxyphosphinyl]amino]-2-phenylethanoic acid;
2-[[phenylhydroxyphosphinyl]amino]-2-phenylethanoic acid;
2-[[benzylhydroxyphosphinyl]amino]-2-phenylethanoic acid;
2-[[phenylethylhydroxyphosphinyl]amino]-2-phenylethanoic acid;
2-[[phenylpropylhydroxyphosphinyl]amino]-2-phenylethanoic acid;
2-[[phenylbutylhydroxyphosphinyl]amino]-2-phenylethanoic acid;
2-[[(2,3,4-trimethoxyphenyl)-3-hydroxyphosphinyl]amino]2-phenylethanoic acid;
2-[[(1-naphthyl)hydroxyphosphinyl]amino]-2-phenylethanoic acid;
2-[[(2-naphthyl)hydroxyphosphinyl]amino]-2-phenylethanoic acid;
2-[[(1-naphthyl)methylhydroxyphosphinyl]amino]-2-phenylethanoic acid;
2-[[(2-naphthyl)methylhydroxyphosphinyl]amino]-2-phenylethanoic acid;
2-[[(1-naphthyl)ethylhydroxyphosphinyl]amino]-2-phenylethanoic acid;
2-[[(2-naphthyl)ethylhydroxyphosphinyl]amino]-2-phenylethanoic acid;
2-[[(1-naphthyl)propylhydroxyphosphinyl]amino]-2-phenylethanoic acid;
2-[[(2-naphthyl)propylhydroxyphosphinyl]amino]-2-phenylethanoic acid;
2-[[(1-naphthyl)butylhydroxyphosphinyl]amino]-2-phenylethanoic acid;
2-[[(2-naphthyl)butylhydroxyphosphinyl]amino]-2-phenylethanoic acid;
2-[[phenylprop-2-enylhydroxyphosphinyl]amino]-2-phenylethanoic acid;
2-[[benzylhydroxyphosphinyl]amino]-2-methylethanoic acid;
2-[[benzylhydroxyphosphinyl]amino]-2-ethylethanoic acid;
2-[[benzylhydroxyphosphinyl]amino]-2-propylethanoic acid;
2-[[benzylhydroxyphosphinyl]amino]-2-butylethanoic acid;
2-[[benzylhydroxyphosphinyl]amino]-2-cyclohexylethanoic acid;
2-[[benzylhydroxyphosphinyl]amino]-2-(cyclohexyl)methylethanoic acid;
2-[[benzylhydroxyphosphinyl]amino]-2-phenylethanoic acid;
2-[[benzylhydroxyphosphinyl]amino]-2-benzylethanoic acid;
2-[[benzylhydroxyphosphinyl]amino]-2-phenylethylethanoic acid;
2-[[benzylhydroxyphosphinyl]amino]-2-phenylpropylethanoic acid;
2-[[benzylhydroxyphosphinyl]amino]-2-phenylbutylethanoic acid;
2-[[benzylhydroxyphosphinyl]amino]-2-(2,3,4-trimethoxyphenyl)ethanoic acid;
2-[[benzylhydroxyphosphinyl]amino]-2-(1-naphthyl)ethanoic acid;
2-[[benzylhydroxyphosphinyl]amino]-2-(2-naphthyl)ethanoic acid;
2-[[benzylhydroxyphosphinyl]amino]-2-(1-naphthyl)methylethanoic acid;
2-[[benzylhydroxyphosphinyl]amino]-2-(2-naphthyl)methylethanoic acid;
2-[[benzylhydroxyphosphinyl]amino]-2-(1-naphthyl)ethylethanoic acid;
2-[[benzylhydroxyphosphinyl]amino]-2-(2-naphthyl)ethylethanoic acid;
2-[[benzylhydroxyphosphinyl]amino]-2-(1-naphthyl)propylethanoic acid;
2-[[benzylhydroxyphosphinyl]amino]-2-(2-naphthyl)propylethanoic acid;
2-[[benzylhydroxyphosphinyl]amino]-2-(1-naphthyl)butylethanoic acid;
2-[[benzylhydroxyphosphinyl]amino]-2-(2-naphthyl)butylethanoic acid;
2-[[benzylhydroxyphosphinyl]amino]-2-phenolprop-2-enylethanoic acid;
2-[[(2-pyridyl)methylhydroxyphosphinyl]amino]-2-phenylethanoic acid;
2-[[(3-pyridyl)methylhydroxyphosphinyl]amino]-2-phenylethanoic acid;
2-[[(4-pyridyl)methylhydroxyphosphinyl]amino]-2-phenylethanoic acid;
2-[[(3-pyridyl)ethylhydroxyphosphinyl]amino]-2-phenylethanoic acid;
2-[[(3-pyridyl)propylhydroxyphosphinyl]amino]-2-phenylethanoic acid;
2-[[(tetrahydrofuranyl)methylhydroxyphosphinyl]amino]-2-phenylethanoic acid;
2-[[(tetrahydrofuranyl)ethylhydroxyphosphinyl]amino]-2-phenylethanoic acid;
2-[[(tetrahydrofuranyl)propylhydroxyphosphinyl]amino]-2-phenylethanoic acid;
2-[[(2-indolyl)methylhydroxyphosphinyl]amino]-2-phenylethanoic acid;
2-[[(3-indolyl)methylhydroxyphosphinyl]amino]-2-phenylethanoic acid;
2-[[(4-indolyl)methylhydroxyphosphinyl]amino]-2-phenylethanoic acid;
2-[[(3-indolyl)ethylhydroxyphosphinyl]amino]-2-phenylethanoic acid;
2-[[(3-indolyl)propylhydroxyphosphinyl]amino]-2-phenylethanoic acid;
2-[[(2-thienyl)methylhydroxyphosphinyl]amino]-2-phenylethanoic acid;
2-[[(3-thienyl)methylhydroxyphosphinyl]amino]-2-phenylethanoic acid;
2-[[(4-thienyl)methylhydroxyphosphinyl]amino]-2-phenylethanoic acid;
2-[[(3-thienyl)ethylhydroxyphosphinyl]amino]-2-phenylethanoic acid;
2-[[(3-thienyl)propylhydroxyphosphinyl]amino]-2-phenylethanoic acid;
2-[[benzylhydroxyphosphinyl]amino]-2-(2-pyridyl)methylethanoic acid;
2-[[benzylhydroxyphosphinyl]amino]-2-(3-pyridyl)methylethanoic acid;
2-[[benzylhydroxyphosphinyl]amino]-2-(4-pyridyl)methylethanoic acid;
2-[[benzylhydroxyphosphinyl]amino]-2-(3-pyridyl)ethylethanoic acid;
2-[[benzylhydroxyphosphinyl]amino]-2-(3-pyridyl)propylethanoic acid;
2-[[benzylhydroxyphosphinyl]amino]-2-(tetrahydrofuranyl)methylethanoic acid;
2-[[benzylhydroxyphosphinyl]amino]-2-(tetrahydrofuranyl)ethylethanoic acid;
2-[[benzylhydroxyphosphinyl]amino]-2-(tetrahydrofuranyl)propylethanoic acid;
2-[[benzylhydroxyphosphinyl]amino]-2-(2-indolyl)methylethanoic acid;
2-[[benzylhydroxyphosphinyl]amino]-2-(3-indolyl)methylethanoic acid;
2-[[benzylhydroxyphosphinyl]amino]-2-(4-indolyl)methylethanoic acid;
2-[[benzylhydroxyphosphinyl]amino]-2-(3-indolyl)ethylethanoic acid;
2-[[benzylhydroxyphosphinyl]amino]-2-(3-indolyl)propylethanoic acid;
2-[[benzylhydroxyphosphinyl]amino]-2-(2-thienyl)methylethanoic acid;
2-[[benzylhydroxyphosphinyl]amino]-2-(3-thienyl)methylethanoic acid;
2-[[benzylhydroxyphosphinyl]amino]-2-(4-thienyl)methylethanoic acid;
2-[[benzylhydroxyphosphinyl]amino]-2-(3-thienyl)ethylethanoic acid; and
2-[[benzylhydroxyphosphinyl]amino]-2-(3-thienyl)propylethanoic acid.

### PMSA Ligands (E.1)

In various embodiments, the PSMA ligands represented by variable A in Structural Formulas A1, A2, A3, A11, A12, and A14 can be those described in U.S. Published Patent Application No. US2001/0044459, or the patents and applications to which it claims priority, U.S. Pat. Nos. 6,271,245 and 5,962,521. The entire teachings of each of these documents are incorporated herein by reference. The variables and terms in this section can be as described herein, more typically as described in this section, or in preferred embodiments can be as described in the documents incorporated by reference in this paragraph.

In various embodiments, the PSMA ligands represented by variable A in Structural Formulas A1, A2, A3, A11, A12, and A14 can be represented by the following Structural Formula:

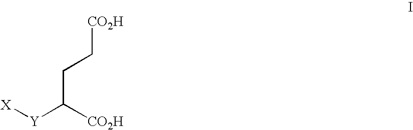

wherein:

X is

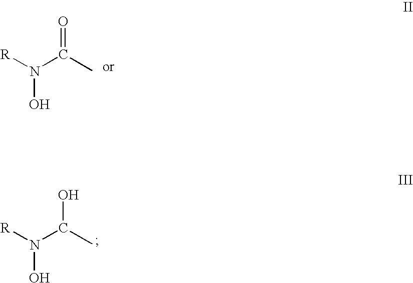

Y is CR₁R₂, NR₃ or O;

R, R₁, R₂ and R₃ are independently selected from the group consisting of hydrogen, C₁-C₉ straight or branched chain alkyl, C₂-C₉ straight or branched chain alkenyl, C₃-C₈ cycloalkyl, C₅-C₇ cycloalkenyl and Ar; and

Ar is selected from the group consisting of 1-naphthyl, 2-naphthyl, 2-indolyl, 3-indolyl, 2-furyl, 3-furyl, 2-thienyl, 3-thienyl, 2-pyridyl, 3-pyridyl, 4-pyridyl, benzyl and phenyl, said Ar having one to three substituent(s) independently selected from the group consisting of hydrogen, halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy and amino.

In various embodiments, the PSMA ligands represented by variable A in Structural Formulas A1, A2, A3, A11, A12, and A14 can be selected from the group consisting of:
2-[[(N-hydroxy)carbamoyl]methyl]pentanedioic acid;
2-[[(N-hydroxy-N-methyl)carbamoyl]methyl]pentanedioic acid;
2-[E(N-butyl-N-hydroxy)carbamoyl]methyl]pentanedioic acid;
2-[((N-benzyl-N-hydroxy)carbamoyl]methyl]pentanedioic acid;
2-[[(N-hydroxy-N-phenyl)carbamoyl]methyl]pentanedioic acid;
2-[[(N-hydroxy-N-2-phenylethyl)carbamoyl]methyl]pentanedioic acid;
2-[[(N-ethyl-N-hydroxy)carbamoyl]methyl]pentanedioic acid;
2-[[(N-hydroxy-N-propyl)carbamoyl]methyl]pentanedioic acid;
2-[[(N-hydroxy-N-3-phenylpropyl)carbamoyl]methyl]pentanedioic acid;
2-([(N-hydroxy-N-4-pyridyl)carbamoyl]methyl]pentanedioic acid;
2-[[(N-hydroxy)carboxamido]methyl]pentanedioic acid;
2-[[N-hydroxy(methyl)carboxamido]methyl]pentanedioic acid;
2-([N-hydroxy(benzyl)carboxamido]methyl]pentanedioic acid;
2-[[N-hydroxy(phenyl)carboxamido]methyl]pentanedioic acid;
2-[[N-hydroxy(2-phenylethyl)carboxamido]methyl]pentanedioic acid;
2-([N-hydroxy(ethyl)carboxamido]methyl]pentanedioic acid;
2-[[N-hydroxy(propyl)carboxamido]methyl]pentanedioic acid;
2-[[N-hydroxy(3-phenylpropyl)carboxamido]methyl]pentanedioic acid; and
2-[[N-hydroxy(4-pyridyl)carboxamido]methyl]pentanedioic acid.

### PMSA Ligands (E.2)

In various embodiments, the PSMA ligands represented by variable A in Structural Formulas A1, A2, A3, A11, A12, and A14 can be those described in U.S. Pat. No. 5,902,817. The entire teachings of this documents are incorporated herein by reference. The variables and terms in this section can be as described herein, more typically as described in this section, or in preferred embodiments can be as described in the documents incorporated by reference in this paragraph.

In various embodiments, the PSMA ligands represented by variable A in Structural Formulas A1, A2, A3, A11, A12, and A14 can be represented by the following Structural Formula:

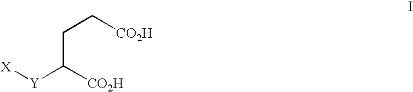

wherein:

X is selected from the group consisting of

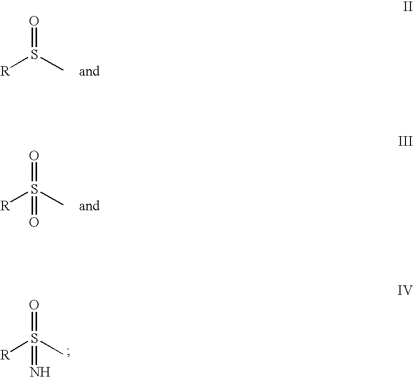

Y is CR₁R₂, NR₃ or O;

R, R₁, R₂ and R₃ are independently selected from the group consisting of hydrogen, C₁-C₉ straight or branched chain alkyl, C₂-C₉ straight or branched chain alkenyl, C₃-C₈ cycloalkyl, C₅-C₇ cycloalkenyl and Ar, wherein said R, R₁, R₂ and R₃ are independently unsubstituted or substituted with C₃-C₈ cycloalkyl, C₅-C₇ cycloalkenyl, halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, amino, Ar or mixtures thereof; and

Ar is selected from the group consisting of 1-naphthyl, 2-naphthyl, 2-indolyl, 3-indolyl, 2-furyl, 3-furyl, 2-thienyl, 3-thienyl, 2-pyridyl, 3-pyridyl, 4-pyridyl, benzyl and phenyl, said Ar having one to three substituent(s) independently selected from the group consisting of hydrogen, halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, amino and mixtures thereof.

In various embodiments, the PSMA ligands represented by variable A in Structural Formulas A1, A2, A3, A11, A12, and A14 can selected from the group consisting of:
2-[(sulfinyl)methyl]pentanedioic acid;
2-[(methylsulfinyl)methyl]pentanedioic acid;
2-[(ethylsulfinyl)methyl]pentanedioic acid;
2-[(propylsulfinyl)methyl]pentanedioic acid;
2-[(butylsulfinyl)methyl]pentanedioic acid;
2-[(phenylsulfinyl)methyl]pentanedioic acid;
2-[[(2-phenylethyl)sulfinyl]methyl]pentanedioic acid;
2-[[(3-phenylpropyl)sulfinyl]methyl]pentanedioic acid;
2-[[(4-pyridyl)sulfinyl]methyl]pentanedioic acid;
2-[(benzylsulfinyl)methyl]pentanedioic acid;
2-[(sulfonyl)methyl]pentanedioic acid;
2-[(methylsulfonyl)methyl]pentanedioic acid;
2-[(ethylsulfonyl)methyl]pentanedioic acid;
2-[(propylsulfonyl)methyl]pentanedioic acid;
2-[(butylsulfonyl)methyl]pentanedioic acid;
2-[(phenylsulfonyl)methyl]pentanedioic acid;
2-[[(2-phenylethyl)sulfonyl]methyl]pentanedioic acid;
2-[[(3-phenylpropyl)sulfonyl]methyl]pentanedioic acid;
2-[[(4-pyridyl)sulfonyl]methyl]pentanedioic acid;
2-[(benzylsulfonyl)methyl]pentanedioic acid;
2-[(sulfoximinyl)methyl]pentanedioic acid;
2-[(methylsulfoximinyl)methyl]pentanedioic acid;
2-[(ethylsulfoximinyl)methyl]pentanedioic acid;
2-[(propylsulfoximinyl)methyl]pentanedioic acid;
2-[(butylsulfoximinyl)methyl]pentanedioic acid;
2-[(phenylsulfoximinyl)methyl]pentanedioic acid;
2-[[(2-phenylethyl)sulfoximinyl]methyl]pentanedioic acid;
2-[[(3-phenylpropyl)sulfoximinyl]methyl]pentanedioic acid;
2-[[(4-pyridyl)sulfoximinyl]methyl]pentanedioic acid; and
2-[(benzylsulfoximinyl)methyl]pentanedioic acid.

### PMSA Ligands (F.1)

In various embodiments, the PSMA ligands represented by variable A in Structural Formulas A1, A2, A3, A11, A12, and A14 can be those described in U.S. Published Patent Application No. US2003/0083505, or the patents and applications to which it claims priority, U.S. Pat. No. 6,452,044 and U.S. Provisional Patent Application No. 60/207,402. The entire teachings of each of these documents are incorporated herein by reference. The variables and terms in this section can be as described herein, more typically as described in this section, or in preferred embodiments can be as described in the documents incorporated by reference in this paragraph.

In various embodiments, the PSMA ligands represented by variable A in Structural Formulas A1, A2, A3, A11, A12, and A14 can be represented by the following Structural Formula:

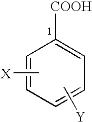

wherein:

X is —W-Z;

W is a bond or a linking group;

Z is a terminal group; and

Y is —COOH oriented meta or para relative to C-1.

Linking groups include without limitation divalent hydrocarbon chains, ethers, sulfides and amines, wherein the hydrocarbon chain, whether alone or part of the ether, sulfide or amine, may be saturated or unsaturated, straight or branched, open or closed, unsubstituted or substituted with one or more substituent(s), preferably, independently selected from the group consisting of C₁-C₆ alkoxy, C₂-C₆ alkenyloxy, phenoxy, benzyloxy, hydroxy, carboxy, carbamido, carbamoyl, carbamyl, carbonyl, carbozoyl, amino, hydroxyamino, formamido, formyl, guanyl, cyano, cyanoamino, isocyano, isocyanato, diazo, azido, hydrazino, triazano, nitrilo, isonitrilo, nitro, nitroso, isonitroso, nitrosamino, imino, nitrosimino, oxo, C₁-C₆ alkylthio, sulfamino, sulfamoyl, sulfeno, sulfhydryl, sulfinyl, sulfo, sulfonyl, sulfoxy, thiocarboxy, thiocyano, isothiocyano, thioformamido, halo, haloalkyl, chlorosyl, chloryl, perchloryl, trifluoromethyl, iodosyl, iodyl, phosphino, phosphinyl, phospho, phosphono, arsino, selanyl, diselanyl, siloxy, silyl and silylene.

Preferably, W is a bond, —(CR₁R₂)ₙ—, —(CR₁R₂)ₙO(CR₃R₄)ₘ—, —(CR₁R₂)ₙS(CR₃R₄)ₘ— or —(CR₁R₂)ₙNR(CR₃R₄)ₘ—, wherein m and n are independently 0-9, and R, R₁, R₂, R₃ and R₄ are independently hydrogen, C₁-C₆ alkyl, C₂-C₆ alkenyl, C₂-C₆ alkynyl, C₆-C₁4 aryl, heteroaryl, C₆-C₁4 carbocycle, heterocycle, halo, hydroxy, sulfhydryl, nitro, amino or C₁-C₆ alkoxy, and said alkyl, alkenyl, alkynyl, aryl, heteroaryl, carbocycle, heterocycle or alkoxy is independently unsubstituted or substituted with one or more substituent(s). More preferably, R, R₁, R₂, R₃ and R₄ are each hydrogen and the total number of carbon atoms in W is 2-6.

Preferably, Z is a metal binding group. More preferably, Z is —COOH, —COR₅, —OR₅, —CF₃, —CN, —F, —Cl, —Br, —I, —NO, —NO₂, —C(O)(NR₅OR₆), —C(O)(NR₅PO₃H₂), —C(O)(NR₅R₆), ═NOH, —NR₅(P(O)(R₆)OH), ═NR₅, —N═NR₅, —N(R₅)CN, —NR(CR₆R₇)ₚCOOH, —NR(CO)NR₆R₇, —NR(COOR₆), —NR₅(CO)R₆, —NR₅(OR₆), —NR₅R₆, —NR₅(SO₂R₆), —O(CO)R₅, —OR₅, —SO₂(OR₅), —SO₂(NR₅R₆), —SO₂R₅, —SO₃R₅, —SNR₅(OR₆), —S(NR₅R₆), —SR₅, —SSR₅, —P(O)(OH)OR₅, —P(O)(OH)R₅ or —PR₅R₆, wherein p is 0-6, and R₅, R₆ and R₇ are independently hydrogen, C₁-C₉ alkyl, C₂-C₉ alkenyl, C₂-C₉ alkynyl, C₆-C₁4 aryl, heteroaryl, C₆-C₁4 carbocycle, heterocycle, halo, hydroxy, sulfhydryl, nitro, amino or C₁-C₉ alkoxy, and said alkyl, alkenyl, alkynyl, aryl, heteroaryl, carbocycle, heterocycle or alkoxy is independently unsubstituted or substituted with one or more substituent(s). More preferably, Z is —NH(CR₆R₇)ₚCOOH, —PO(OH)OR₅, —PO(OH)R₅, —NR₅(P(O)(OH)R₆), —CON(R₅)(OH) or —SH.

In a preferred embodiment of formula I, X is —SO₂-aryl, carboxy, S-aryl, nitro, halo, amino, —SO₃H, —(CR₁R₂)ₙCO₂R₃, —NR₅(CR₁R₂)ₙCO₂R₃, —(C═O)-aryl, —(C═O)-phenyoxy-aryl, —(C═O)N₅-aryl, —O(CR₁R₂)ₙ—S—S—(CR₃R₄)ₘO-aryl, hydroxy, —(CR₁R₂)ₙNR₅(CR₃R₄)ₘ-heteroaryl, —NR₅—(C═O)alkyl, and NR₅—(C═O)-aryl; wherein m and n are independently 0-9; R₁, R₂, R₃, R₄, and R₅ are independently hydrogen C₁-C₆ alkyl, C₂-C₆ alkenyl, C₂-C₆ alkynyl, C₆-C₁4 aryl, heteoaryl, C₆-C₁4 carbocycle, heterocycle, halo, hydroxy, sulfhydryl, nitro, amino or C₁-C₆ alkoxy, wherein said alkyl, alkenyl, alkynyl, aryl, heteroaryl, carbocycle, heterocycle or alkoxy is independently unsubstituted or substituted with one or more substituent(s).

In various embodiments, the PSMA ligands represented by variable A in Structural Formulas A1, A2, A3, A11, A12, and A14 can be selected from the group consisting of:
2-[(4-carboxyphenyl)-sulfonyl]-1,4-benzenedicarboxylic acid (1)
2-[(2,5-dicarboxyphenyl)-sulfonyl]-1,4-benzenedicarboxylic acid (2)
1,2,4-benzenetricarboxylic acid (3)
2-[(2-carboxyphenyl)thio]-1,4-benzenedicarboxylic acid (4)
2-nitro-1,4-benzenedicarboxylic acid (5)
2-bromo-1,4-benzenedicarboxylic acid (6)
2-amino-1,4-benzenedicarboxylic acid (7)
2-sulfoterephthalic acid, monosodium salt (8)
2-carboxymethyl-14-benzenedicarboxylic acid (9)
2-[(2-furanylmethyl)-amino]-1,4-benzenedicarboxylic acid (10)
2-[(carboxymethyl)amino]-1,4-benzenedicarboxylic acid (11)

In various embodiments, the PSMA ligands represented by variable A in Structural Formulas A1, A2, A3, A11, A12, and A14 can be selected from the group consisting of:
4-(4-nitrobenzoyl)-1,3-benzenedicarboxylic acid (12)
4-[4-(2,4-dicarboxybenzoyl)phenoxy]-1,2-benzenedicarboxylic acid (13)
4-[[(2,4,6-trimethylphenyl)aimno carbonyl]-1,3-benzenedicarboxylic acid (14)
4-nitro-1,3-benzenedicarboxylic acid (15)
4-[(1-naphthalenylamino)-carbonyl]-1,3-benzenedicarboxylic acid (16)
1,2,4-benzenetricarboxylic acid (17)
4-[(2-carboxyphenyl)thio]-1,3-benzenedicarboxylic acid (18)
4-[3-[[3-(2,4-dicarboxyphenoxy)propyl]-dithio]propoxy]-1,3-benzenedicarboxylic acid (19)
4-hydroxy-1,3-benzenedicarboxylic acid (20)
4-[(2-furanylmethyl)amino]-1,3-benzenedicarboxylic acid (21)
4-(2-mercaptoethyl)-1,3-benzenedicarboxylic acid (22)

In various embodiments, the PSMA ligands represented by variable A in Structural Formulas A1, A2, A3, A11, A12, and A14 can be selected from the group consisting of:
5-[4,5-dihydro-5-(4-hydroxyphenyl)-3-phenyl-1H-pyrazol-1-yl]-1,3-benzenedicarboxylic acid (23);
5-(4,5-dihydro-3-methyl-5-phenyl-1H-pyrazol-1-yl)-1,3-benzenedicarboxylic acid (24);
5-[[(4-chloro-3-nitrophenyl)amino]sulfonyl]-1,3-benzenedicarboxylic acid (25);
5-[[[4-chloro-3-[[3-(2-methoxyphenyl)-1,3-dioxopropyl]amino]phenyl]amino]sulfon yl-1,3-benzenedicarboxylic acid (26);
5-[[3-[4-(acetylamino)phenyl]-1,3-dioxopropyl]amino]-1,3-benzenedicarboxylic acid (27);
5-acetylamino-1,3-benzenedicarboxylic acid (28);
5-[[(1-hydroxy-2-naphthalenyl)carbonyl]-methylamino]-1,3-benzenedicarboxylic acid (29);
5-(4-carboxy-2-nitrophenoxy)-1,3-benzenedicarboxylic acid (30);
5-sulfo-1,3-benzenedicarboxylic acid (31);
5-nitro-1,3-benzenedicarboxylic acid (32);
5-amino-1,3-benzenedicarboxylic acid (33);
1,3,5-benzenetricarboxylic acid (34);
5-[[3-amino-4-chlorophenyl)amino]sulfonyl]-1,3-benzenedicarboxylic acid (35);
5-(3-mercaptopropoxy)-1,3-benzenedicarboxylic acid (36);
5-hydroxy-1,3-benzenedicarboxylic acid (37);
5-(2-mercaptoethoxy)-1,3-benzenedicarboxylic acid (38);
5-[(hydroxyamino)-carbonyl]-1,3-benzenedicarboxylic acid (39);
5-phosphono-1,3-benzenedicarboxylic acid (40);
5-mercaptomethyl-1,3-benzenedicarboxylic acid (41);
5-phosphonomethyl-1,3-benzenedicarboxylic acid (42);
5-[[(carboxymethyl)amino]-methyl]-1,3-benzenedicarboxylic acid (43);
5-[(carboxymethyl)amino]-1,3-benzenedicarboxylic acid (44);
5-[[(2-furanylmethyl)amino]-methyl]-1,3-benzenedicarboxylic acid (45);
5-[2-(hydroxyamino)-2-oxoethyl]-1,3-benzenedicarboxylic acid (46); and
5-(2-mercaptoethyl)-1,3-benzenedicarboxylic acid (47).

### PMSA Ligands (F.2)

In various embodiments, the PSMA ligands represented by variable A in Structural Formulas A1, A2, A3, A11, A12, and A14 can be those described in U.S. Published Patent Application No. US2003/0087897, or the patents and applications to which it claims priority, U.S. Provisional Patent Application Nos. 60/290,015 and 60/342,741. The entire teachings of each of these documents are incorporated herein by reference. The variables and terms in this section (F.2) can be as described herein, more typically as described in this section, or in preferred embodiments can be as described in the documents incorporated by reference in this paragraph.

In various embodiments, the PSMA ligands represented by variable A in Structural Formulas A1, A2, A3, A11, A12, and A14 can be represented by the following Structural Formula:

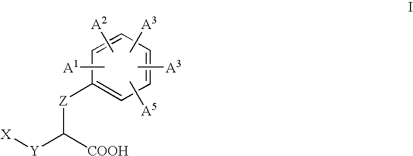

wherein:

X is —(CO)NHOH or —N(OH)CHO;

Y is a bond or a divalent linking group having from 1 to 9 carbon atom(s) and from 0 to 5 heteroatom(s) independently selected from the group consisting of oxygen, sulfur and nitrogen;

Z is —CR₁R₂—, —NR₁—, —O— or —S—;

A₁, A₂, A₃, A₄ and A₅ are independently hydrogen, C₁-C₉ alkyl, C₂-C₉ alkenyl, C₂-C₉ alkynyl, aryl, heteroaryl, carbocycle, heterocycle, C₁-C₉ alkoxy, C₂-C₉ alkenyloxy, phenoxy, benzyloxy, hydroxy, halo, nitro, cyano, isocyano, —COOR₃, —COR₃, —NR₃R₄, —SR₃, —SOR₃, —SO₂R₃, —SO₂(OR₃), —(CO)NR₃R₄, —(CO)NR₃(CH₂)ₙCOOH, —NR₃(CO)R₄ or —(CH₂)ₙCOOH, or any adjacent two of A₁, A₂, A₃ and A₄ form with the benzene ring a fused ring that is saturated or unsaturated, aromatic or non-aromatic, and carbocyclic or heterocyclic, said heterocyclic ring containing 1 or 2 oxygen, nitrogen and/or sulfur heteroatom(s);

n is 1-3;

R₁, R₂, R₃ and R₄ are independently hydrogen, C₁-C₉ alkyl, C₂-C₉ alkenyl, C₂-C₉ alkynyl, aryl, heteroaryl, carbocycle or heterocycle; and

said alkyl, alkenyl, alkynyl, aryl, heteroaryl, carbocycle, heterocycle, alkoxy, alkenyloxy, phenoxy, benzyloxy, and fused ring are independently unsubstituted or substituted with one or more substituent(s).

In various embodiments, the PSMA ligands represented by variable A in Structural Formulas A1, A2, A3, A11, A12, and A14 can be represented by the following Structural Formula:

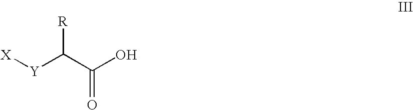

wherein:

X is —(CO)NHOH or —N(OH)CHO;

Y is a bond or a divalent linking group having from 1 to 9 carbon atom(s) and from 0 to 5 heteroatom(s) independently selected from the group consisting of oxygen, sulfur and nitrogen; and

R is hydrogen, C₁-C₉ alkyl, C₂-C₉ alkenyl, C₂-C₉ alkynyl, C₁-C₉ alkoxy or C₂-C₉ alkenoxy, wherein said alkyl, alkenyl, alkynyl, alkoxy and alkenoxy are independently unsubstituted or substituted with one or more substituent(s); provided that when Y is methylene, amine or oxygen, then R is not carboxyethyl.

In various embodiments, the PSMA ligands represented by variable A in Structural Formulas A1, A2, A3, A11, A12, and A14 can be selected from the group consisting of:
3-tert-Butyl-5-(2-carboxy-3-hydroxycarbamoyl-propyl)-benzoic acid;
3-tert-Butyl-5-(2-carboxy-4-hydroxycarbamoyl-butyl)-benzoic acid;
3-(2-Carboxy-4-hydroxycarbamoyl-butyl)-benzoic acid;
3-(2-Carboxy-5-hydroxycarbamoyl-pentyl)-benzoic acid;
3-(2-Carboxy-3-hydroxycarbamoyl-propyl)-benzoic acid;
3-(2-Carboxy-2-hydroxycarbamoyl-ethyl)-benzoic acid;
3-tert-Butyl-5-(2-carboxy-2-hydroxycarbamoyl-ethyl)-benzoic acid;

### 3-tert-Butyl-5-(2-carboxy-2-hydroxycarbamoyl-ethyl)-benzoic acid methyl ester;

3-(2-Carboxy-3-hydroxyamino-propyl)-benzoic acid;
3-(2-Carboxy-2-hydroxycarbamoy-1-ethyl)-benzoic acid methyl ester;
3-(2-Carboxy-5-hydroxycarbamoylmethyl-sulfanylpentyl)-benzoic acid;
3-[2-Carboxy-5-(2-hydroxycarbamoyl-ethylsulfanyl)-pentyl]-benzoic acid;
3-[2-Carboxy-5-(1-hydroxycarbamoyl-propylsulfanyl)-pentyl]-benzoic acid;
3-tert-Butyl-5-(2-carboxy-4-hydroxycarbamoylmethyl-sulfanylbutyl)-benzoic acid;
3-[2-Carboxy-5-(hydroxy-carbamoyl-phenyl-methylsulfanyl)-pentyl]-benzoic acid;
3-[2-Carboxy-5-(1-hydroxycarbamoyl-butylsulfanyl)-pentyl]-benzoic acid;
5-(2-Carboxy-5-hydroxycarbamoylmethyl-sulfanylpentyl)-biphenyl-3-carboxylic acid;
3-Bromo-5-(2-carboxy-5-hydroxycarbamoylmethyl-sulfanylpentyl)-benzoic acid;
3-Benzyloxy-5-(2-carboxy-5-hydroxycarbamoylmethyl-sulfanylpentyl)-benzoic acid;
3-[2-Carboxy-5-(1-hydroxycarbamoyl-2-methyl-propylsulfanyl)-pentyl]-benzoic acid;
3-(2-Carboxy-3-hydroxycarbamoylmethyl-sulfanylpropyl)-benzoic acid;
3-(2-Carboxy-5-hydroxycarbamoylmethyl-sulfanylpentyl)-5-phenoxy-benzoic acid;
3-(2-Carboxy-6-hydroxycarbamoylmethyl-sulfanylhexyl)-benzoic acid;
3-(2-Carboxy-4-hydroxycarbamoylmethyl-sulfanylbutyl)-benzoic acid;
3-[2-Carboxy-3-(3-hydroxycarbamoyl-propylsulfanyl)-propyl]-benzoic acid;
3-[2-Carboxy-5-(4-hydroxycarbamoyl-butylsulfanyl)-pentyl]-benzoic acid;
3-{2-Carboxy-5-[(hydroxy-methyl-carbamoyl)-methylsulfanyl]-pentyl}-benzoic acid;
3-tert-Butyl-5-[2-carboxy-4-(1-hydroxycarbamoyl-propylsulfanyl)-butyl]-benzoic acid;
3-(2-Carboxy-5-hydroxycarbamoylmethyl-sulfanylpentyl)-4-chloro-benzoic acid;
3-[2-Carboxy-4-(1-hydroxycarbamoyl-propylsulfanyl)-butyl]-benzoic acid;
3-[2-Carboxy-3-(1-hydroxycarbamoyl-propylsulfanyl)-propyl]-benzoic acid;
2-Biphenyl-3-ylmethyl-5-hydroxycarbamoylmethylsulfanyl-pentanoic acid;
3′-(2-Carboxy-5-hydroxycarbamoylmethylsulfanyl-pentyl)-biphenyl-3-carboxylic acid;
2-(3-Hydroxycarbamoyl-methylsulfanylpropyl)-pentanedioic acid;
3-(2-Carboxy-5-{[(hydroxy-amino)carbonyl]amino}-pentyl)-5-tert-butylbenzoic acid;
2-Bromo-4-(2-carboxy-5-hydroxycarbamoylmethyl-sulfanylpentyl)-benzoic acid;

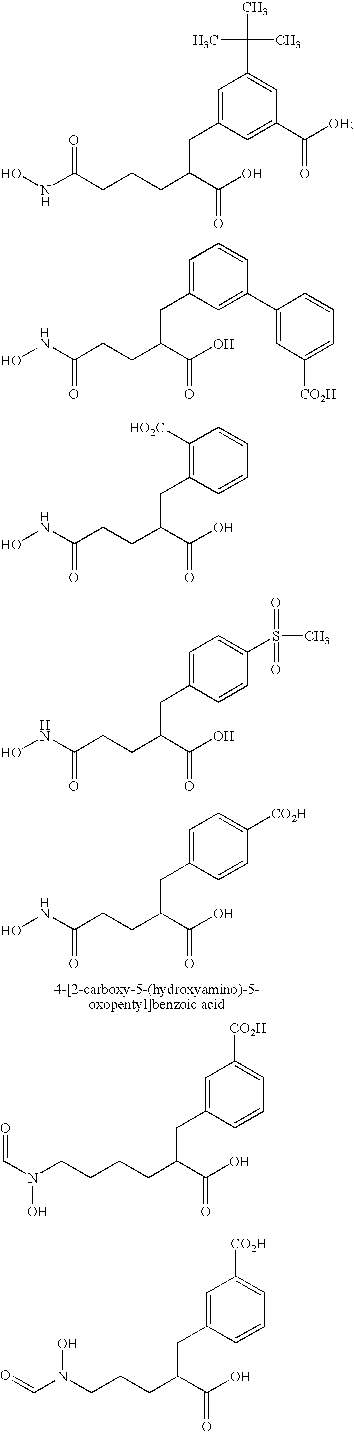

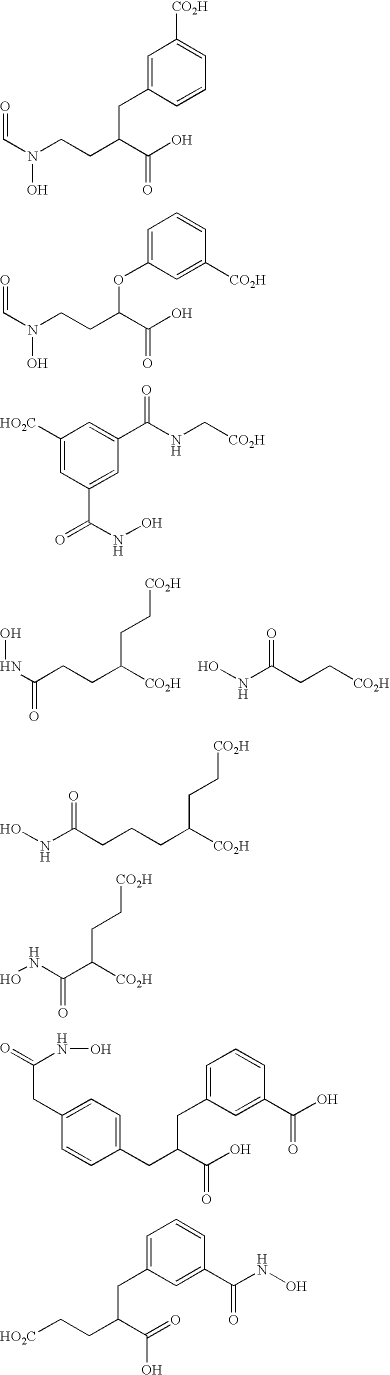

### PMSA Ligands (F.3)

In various embodiments, the PSMA ligands represented by variable A in Structural Formulas A1, A2, A3, A11, A12, and A14 can be those described in U.S. Published Patent Application No. US2004/0198824, or the patents and applications to which it claims priority, U.S. Pat. No. 6,740,777, and U.S. Provisional Patent Application Nos. 60/294,036 and 60/342,746. The entire teachings of each of these documents are incorporated herein by reference. The variables and terms in this section can be as described herein, more typically as described in this section, or in preferred embodiments can be as described in the documents incorporated by reference in this paragraph.

In various embodiments, the PSMA ligands represented by variable A in Structural Formulas A1, A2, A3, A11, A12, and A14 can be represented by the following Structural Formula:

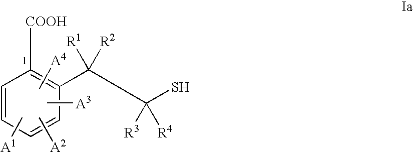

wherein:

R₁, R₂, R₃, and R₄ are independently hydrogen or C₁-C₃ alkyl; and

A₁, A₂, A₃, and A₄ are independently hydrogen, C₁-C₉ alkyl, C₂-C₉ alkenyl, C₂-C₉ alkynyl, aryl, heteroaryl, carbocycle, heterocycle, halo, hydroxy, sulffiydryl, nitro, amino, cyano, isocyano, thiocyano, isothiocyano, formamido, thioformamido, sulfo, sulfino, C₁-C₉ alkylsulfonyl, C₁-C₉ alkoxy, C₂-C₉ alkenoxy, phenoxy, or benzyloxy,

wherein said alkyl, alkenyl, alkynyl, aryl, heteroaryl, carbocycle, heterocycle, alkoxy, alkenoxy, phenoxy, and benzyloxy are independently unsubstituted or substituted with one or more substituent(s).

In one embodiment, R₁, R₂, R₃, R₄, A₂, A₃, and A₄ are hydrogen; and A1 is hydrogen, —(CH₂)ₙ—W, or —Y—(CH₂)ₙ—W, wherein: n is 0-3; Y is O, S, or NR wherein R is hydrogen or C1-C4 alkyl; and W is C1-C6 alkyl or phenyl, wherein W is unsubstituted or substituted with C1-C4 alkyl, C1-C4 alkoxy, carboxy, or halo.

In various embodiments, the PSMA ligands represented by variable A in Structural Formulas A1, A2, A3, A11, A12, and A14 can be represented by the following Structural Formula:

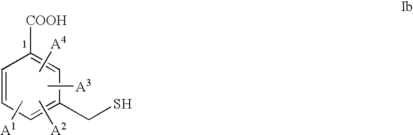

wherein:

A₁, A₂, A₃ and A₄ are independently hydrogen, C₁-C₉ alkyl, C₂-C₉ alkenyl, C₂-C₉ alkynyl, aryl, heteroaryl, carbocycle, heterocycle, halo, hydroxy, sulfhydryl, nitro, amino, cyano, isocyano, thiocyano, isothiocyano, formamido, thioformamido, sulfo, sulfino, C₁-C₉ alkylsulfonyl, C₁-C₉ alkoxy, C₂-C₉ alkenoxy, phenoxy, or benzyloxy,

wherein said alkyl, alkenyl, alkynyl, aryl, heteroaryl, carbocycle, heterocycle, alkoxy, alkenoxy, phenoxy, and benzyloxy are independently unsubstituted or substituted with one or more substituent(s),

wherein if A₁ is chloro, fluoro, amino, or thiomethyl then A₂, A₃, and A₄ may not all be hydrogen,

and wherein at least one of A₁, A₂, A₃, and A₄ is not hydrogen.

In one embodiment, A₂, A₃, and A₄ are hydrogen; and A₁ is —(CH₂)ₙ—Ar or —Y—(CH₂)ₙ—Ar, wherein n is 0-3, Y is O, S, or NR wherein R is hydrogen or C1-C4 alkyl, and Ar is phenyl, unsubstituted or substituted with C1-C4 alkyl, carboxy, or halo.

In various embodiments, the PSMA ligands represented by variable A in Structural Formulas A1, A2, A3, A11, A12, and A14 can be represented by the following Structural Formula:

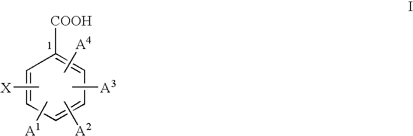

wherein:

X is —(CR₁R₂)ₙSH, —O(CR₁R₂)₂SH, —S(CR₁R₂)₂SH, or —NR(CR₁R₂)₂SH;

n is 1-3; and

R, R₁, R₂, A₁, A₂, A₃ and A₄ are independently hydrogen, C₁-C₉ alkyl, C₂-C₉ alkenyl, C₂-C₈ alkynyl, aryl, heteroaryl, carbocycle, heterocycle, halo, hydroxy, sulfhydryl, nitro, amino, cyano, isocyano, thiocyano, isothiocyano, formamido, thioformamido, sulfo, sulfino, C₁-C₉ alkylsulfonyl, C₁-C₉ alkoxy, C₂-C₉ alkenoxy, phenoxy, or benzyloxy,

wherein said alkyl, alkenyl, alkynyl, aryl, heteroaryl, carbocycle, heterocycle, alkoxy, alkenoxy, phenoxy, and benzyloxy are independently unsubstituted or substituted with one or more substituent(s).

In various embodiments, the PSMA ligands represented by variable A in Structural Formulas A1, A2, A3, A11, A12, and A14 can be selected from the group consisting of:
2-(2-mercaptoethyl)-benzoic acid;

### 5-hydroxy-2-(2-mercaptoethyl)-benzoic acid;

5-[(4-carboxyphenyl)methoxy]-2-(2-mercaptoethyl)-benzoic acid;
2-(2-mercaptoethyl)-5-(phenylmethoxy)-benzoic acid;
2-(carboxymethoxy)-6-(2-mercaptoethyl)-benzoic acid;
5-[(3-carboxyphenyl)methoxy]-2-(2-mercaptoethyl)-benzoic acid;
2-(2-mercaptoethyl)-6-(phenylmethoxy)-benzoic acid;
2-[(2-carboxyphenyl)methoxy]-6-(2-mercaptoethyl)-benzoic acid;
2-[(4-carboxyphenyl)methoxy]-6-(2-mercaptoethyl)-benzoic acid;
3-(2-mercaptoethyl)-[1,1′-biphenyl]-2,3′-dicarboxylic acid;
5-(mercaptomethyl)-2-(2-phenylethoxy)-benzoic acid;
2-(3,3-dimethylbutoxy)-6-(2-mercaptoethyl)-benzoic acid;
2-(2-mercaptoethyl)-6-(2-phenylethoxy)-benzoic acid;
2-[(2-chlorophenyl)methoxy]-6-(2-mercaptoethyl)-benzoic acid;
2-[[3-carboxy-5-(1,1-dimethylethyl)phenyl]methoxy]-6-(2-mercaptoethyl)-benzoic acid;
3-(2-mercaptoethyl)-[1,1′-biphenyl]-2,4′-dicarboxylic acid;
2-[(4-carboxy-2-methoxyphenyl)methoxy]-6-(2-mercaptoethyl)-benzoic acid;
2-[(4-carboxy-3-methoxyphenyl)methoxy]-6-(2-mercaptoethyl)-benzoic acid;
2-[(2-bromo-4-carboxyphenyl)methoxy]-6-(2-mercaptoethyl)-benzoic acid;
2-[(3-bromo-4-carboxyphenyl)methoxy]-6-(2-mercaptoethyl)-benzoic acid 2-(2-mercaptoethyl)-6-phenoxy-benzoic acid;
2-(2-mercaptoethyl)-6-phenylaminobenzoic acid;
2-(2-mercaptoethyl)-6-(phenylthio)-benzoic acid;
5′-(1,1-dimethylethyl)-3-(2-mercaptoethyl)-[1,1′-biphenyl]-2,3′-dicarboxylic acid;
2-bromo-5-(mercaptomethyl)-benzoic acid;
4-(mercaptomethyl)-[1,1′-biphenyl]-2,3′-dicarboxylic acid;
5-(mercaptomethyl)-2-(phenylmethoxy)-benzoic acid;
4-bromo-3-(mercaptomethyl)-benzoic acid;
3-(2-mercaptoethyl)-benzoic acid;
3-(mercaptomethyl)-benzoic acid-2-(mercaptomethyl)-benzoic acid;
2-[(4-chlorophenyl)methoxy]-6-(2-mercaptoethyl)-benzoic acid;
2-(biphenyl-2-ylmethoxy)-6-(2-mercaptoethyl)-benzoic acid;
2-[(3-bromo-5-carboxyphenyl)methoxy]-6-(2-mercaptoethyl)-benzoic acid;
2-[(2-bromo-5-carboxyphenyl)methoxy]-6-(2-mercaptoethyl)-benzoic acid;
2-(2-mercaptoethyl)-6-[(4-methoxyphenyl)methoxy]-benzoic acid;
2-(2-mercaptoethyl)-6-[(4-methylphenyl)methoxy]-benzoic acid;
2-[(4-bromo-3-carboxyphenyl)methoxy]-6-(2-mercaptoethyl)-benzoic acid;
2-[(2-carboxy-5-methoxyphenyl)methoxy]-6-(2-mercaptoethyl)-benzoic acid;
2-(3-carboxy-benzyloxy)-6-(2-mercapto-ethyl)-benzoic acid;
2-(4-bromo-benzyloxy)-6-(2-mercapto-ethyl)-benzoic acid;
2-(4-tert-butyl-benzyloxy)-6-(2-mercaptoethyl)-benzoic acid;
2-(3-bromo-benzyloxy)-6-(2-mercaptoethyl)-benzoic acid;
2-(2-mercapto-ethyl)-6-methoxy-benzoic acid;
2-benzhydryloxy-6-(2-mercapto-ethyl)-benzoic acid;
2-(3-chloro-benzyloxy)-6-(2-mercaptoethyl)-benzoic acid;
3-(2-mercapto-ethyl)-biphenyl-2-carboxylic acid; and
2-carboxymethyl-6-(2-mercapto-ethyl)-benzoic acid.

### PMSA Ligands (F.4)

In various embodiments, the PSMA ligands represented by variable A in Structural Formulas A1, A2, A3, A11, A12, and A14 can be those described in U.S. Published Patent Application No. US2005/0004203, or the patents and applications to which it claims priority, U.S. Provisional Patent Application No. 60/450,648. The entire teachings of each of these documents are incorporated herein by reference The variables and terms in this section can be as described herein, more typically as described in this section, or in preferred embodiments can be as described in the documents incorporated by reference in this paragraph.

In various embodiments, the PSMA ligands represented by variable A in Structural Formulas A1, A2, A3, A11, A12, and A14 can be represented by the following Structural Formula:

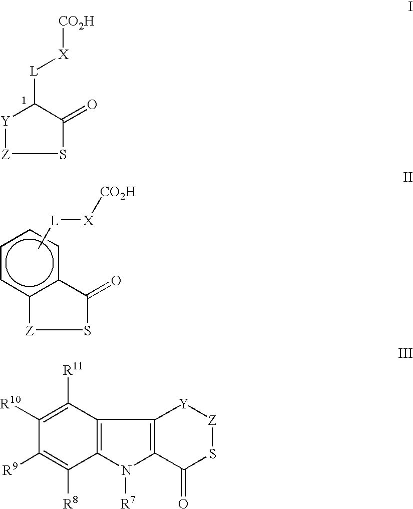

wherein:

X is C₁-C₄ alkylene, C₂-C₄ alkenylene, C₂-C₄ alkynylene, C₃-C₈ cycloalkylene, C₅-C₇ cycloalkenylene or Ar, wherein the alkylene, alkenylene, alkynylene, cycloalkylene or cycloalkenylene is unsubstituted or substituted with one or more substituent(s);

L is a bond, —CR₁R₂—, —O—, —S—, —SO₂— or —NR₁—;

Y is —O—, —S—, —CR₃R₄— or ′NR₃—;

Z is —(CR₅R₆)ₙ—;

n is 1, 2, 3 or 4;

Ar is a bivalent aryl or heteroaryl radical that is unsubstituted or substituted with one or more substituent(s);

R₁, R₂, R₃, R₄, R₅ and R₆ are independently hydrogen, C₁-C₄ alkyl or C₂-C₄ alkenyl, wherein the alkyl or alkenyl is unsubstituted or substituted with one or more substituent(s);

R₇ is hydrogen, phenyl, phenylethyl or benzyl wherein the phenyl, phenylethyl or benzyl is unsubstituted or substituted with one or more substituent(s); and

R₈, R₉, R₁₀ and R₁₁ are independently hydrogen, carboxy, hydroxy, halo, nitro, cyano, C₁-C₄ alkyl or C₁-C₄ alkoxy.

In various embodiments, the PSMA ligands represented by variable A in Structural Formulas A1, A2, A3, A11, A12, and A14 can be selected from the group consisting of:
3-(2-Oxo-tetrahydrothiopyran-3-yl)-propionic acid;
3-[(2-oxotetrahydro-2H-thiopyran-3-yl)methyl]benzoic acid;
3-(1-Oxoisothiochroman-8-yl)-benzoic acid;
3-(1-Oxoisothiochroman-8-yloxymethyl)-benzoic acid; and
3-(1-Oxo-3,4-dihydro-1H-2-thia-9-aza-fluoren-9-yl)-benzoic acid.

### PMSA Ligands (F.5)

In various embodiments, the PSMA ligands represented by variable A in Structural Formulas A1, A2, A3, A11, A12, and A14 can be those described in U.S. Pat. No. 6,812,364, or the patents and applications to which it claims priority, U.S. Pat. No. 6,586,623 and U.S. Provisional Patent Application Nos. 60/261,754 and 60/342,772. The entire teachings of each of these documents are incorporated herein by reference. The variables and terms in this section can be as described herein, more typically as described in this section, or in preferred embodiments can be as described in the documents incorporated by reference in this paragraph.

In various embodiments, the PSMA ligands represented by variable A in Structural Formulas A1, A2, A3, A11, A12, and A14 can be represented by the following Structural Formula:

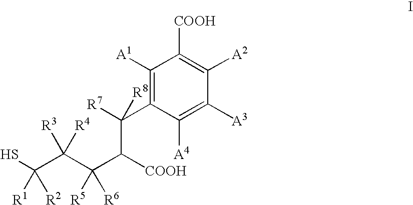

wherein:

R₁, R₂, R₃, R₄, R₅, R₆, R₇ and R₈ are independently hydrogen or C₁-C₃ alkyl;

A₁, A₂, A₃ and A₄ are independently hydrogen, C₁-C₆ alkyl, C₁-C₆ alkoxy, halo, nitro, phenyl, phenoxy, benzyl, benzyloxy or —COOH, or any adjacent two of A₂, A₃ and A₄ form with the benzene ring a fused 5- or 6-membered carbocyclic or heterocyclic aromatic ring, said heterocyclic aromatic ring containing 1 or 2 oxygen, nitrogen and/or sulfur heteroatom(s).

In various embodiments, the PSMA ligands represented by variable A in Structural Formulas A1, A2, A3, A11, A12, and A14 can be represented by the following Structural Formula:

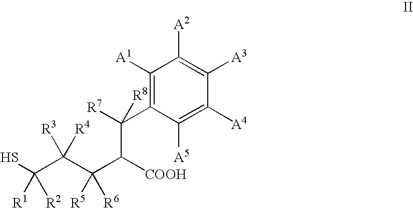

wherein:

R₁, R₂, R₃, R₄, R₅, R₆, R₇ and R₈ are independently hydrogen or C₁-C₃ alkyl; and

A₁, A₂, A₃, A₄ and A₅ are independently hydrogen, C₁-C₆ alkyl, C₁-C₆ alkoxy, C₁-C₃ perhaloalkyl, phenyl, phenoxy, benzyl, benzyloxy, hydroxy, halo, cyano, nitro, —SO₂R₉, —(C═O)NR₉R₁₀, —(C═O)NR₉ (CH₂)ₙCOOH, —NR₉(C═O)R₁₀, —(CH₂)ₙCOOH or —COOH, or any adjacent two of A₁, A₂, A₃, A₄ and A₅ form with the benzene ring a fused 5- or 6-membered carbocyclic or heterocyclic aromatic ring, said heterocyclic aromatic ring containing 1 or 2 oxygen, nitrogen and/or sulfur heteroatom(s);

R₉ and R₁₀ are independently hydrogen, C₁-C₆ alkyl, phenyl or benzyl; and

n is 1-3;

provided that if A₁, A₃ and A₅ are independently hydrogen, C₁-C₆ alkyl, C₁-C₆ alkoxy, halo, nitro, phenyl, phenoxy, benzyl, benzyloxy or —COOH, then neither A₂ nor A4 are —COOH; and provided that if any adjacent two of A₃, A₄ and A₅ form with the benzene ring a fused 5- or 6-membered carbocyclic or heterocyclic aromatic ring, said heterocyclic aromatic ring containing 1 or 2 oxygen, nitrogen and/or sulfur heteroatom(s), then A₂ is not —COOH.

In various embodiments, the PSMA ligands represented by variable A in Structural Formulas A1, A2, A3, A11, A12, and A14 can be represented by the following Structural Formula:

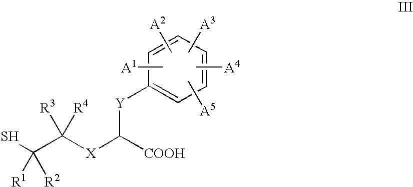

wherein:

X and Y are independently —CR₅R₆—, —O—, —S— or —NR—, provided that at least one of X and Y is/are —CR₅R₆—;

A₁, A₂, A₃, A₄ and A₅ are independently hydrogen, C₁-C₉ alkyl, C₂-C₉ alkenyl, C₂-C₉ alkynyl, aryl, heteroaryl, carbocycle, heterocycle, C₁-C₉ alkoxy, C₂-C₉ alkenyloxy, phenoxy, benzyloxy, hydroxy, halo, nitro, cyano, isocyano, —COOR₇, —COR₇, —NR₇, R₈, —SR₇, —SOR₇, —SO₂R₇, —SO₂ (OR₇), —(C═O)NR₇R₈, —(C═O)NR₇ (CH₂)ₙCOOH, —NR₇ (C═O)R₈ or —(CH₂)ₙCOOH, or any adjacent two of A₁, A₂, A₃, A₄ and A₅ form with the benzene ring a fused ring that is saturated or unsaturated, aromatic or non-aromatic, and carbocyclic or heterocyclic, said heterocyclic ring containing 1 or 2 oxygen, nitrogen and/or sulfur heteroatom(s);

n is 1-3;

R, R₁, R₂, R₃, R₄, R₅, R₆, R₇ and R₈ are independently hydrogen, C₁-C₉ alkyl, C₂-C₉ alkenyl, C₂-C₉ alkynyl, aryl, heteroaryl, carbocycle or heterocycle; and

said alkyl, alkenyl, alkynyl, aryl, heteroaryl, carbocycle, heterocycle, alkoxy, alkenyloxy, phenoxy, benzyloxy, and fused ring are independently unsubstituted or substituted with one or more substituent(s);

provided that if A₁, A₂ and A₃ are each hydrogen, and A₄ and A₅ are each —COOH, then A₄ is ortho to A₅; and provided that if Y is —CR₅R₆—, then at least one of A₁, A₂, A₃, A₄ and A₅ is/are independently phenoxy, benzyloxy, aryl, heteroaryl, carbocycle or heterocycle that is substituted with one or more substituent(s).

In various embodiments, the PSMA ligands represented by variable A in Structural Formulas A1, A2, A3, A11, A12, and A14 can be selected from the group consisting of:
alpha-(3-mercaptopropyl)-3-(trifluoromethyl)-benzenepropanoic acid;
alpha-(3-mercaptopropyl)-benzenepropanoic acid;
4-hydroxy-alpha-(3-mercaptopropyl)-benzenepropanoic acid;
2,3,4,5,6-pentafluoro-alpha-(3-mercaptopropyl)-benzenepropanoic acid;
3-carboxy-alpha-(3-mercaptopropyl)-benzenepropanoic acid;
4-carboxy-alpha-(3-mercaptopropyl)-benzenepropanoic acid;
alpha-(3-mercaptopropyl)-4-(methylsulfonyl)-benzenepropanoic acid;
2-cyano-alpha-(3-mercaptopropyl)-benzenepropanoic acid;
5-(2-carboxy-5-mercaptopentyl)-1,3-benzenedicarboxylic acid;
5-carboxy-2-chloro-alpha-(3-mercaptopropyl)-benzenepropanoic acid;
3-carboxy-4-fluoro-alpha-(3-mercaptopropyl)-benzenepropanoic acid;
4-(2-cyanophenyl)-alpha-(3-mercaptopropyl)-benzenepropanoic acid;
2-(aminocarbonyl)-alpha-(3-mercaptopropyl)-benzenepropanoic acid;
3-(1-carboxy-4-mercaptobutoxy)-benzoic acid;
5-mercapto-2-phenoxy-pentanoic acid;
2-(3,5-dimethoxyphenoxy)-5-mercapto-pentanoic acid;
alpha-(3-mercaptopropyl)-2,5-dimethoxy-benzenepropanoic acid;
alpha-(3-mercaptopropyl)-3-phenoxy-benzenepropanoic acid;
2-(3-hydroxyphenoxy)-5-mercapto-pentanoic acid;
3-(1-carboxy-4-mercaptobutoxy)-benzeneacetic acid;
4-(1-carboxy-4-mercaptobutoxy)-benzeneacetic acid;
5 alpha-(3-mercaptopropyl)-4-phenyl-benzenepropanoic acid;
2-(3-acetylphenoxy)-5-mercapto-pentanoic acid;
2-[3-(acetylamino)phenoxy]-5-mercapto-pentanoic acid;
2-(4-acetylphenoxy)-5-mercaptopentanoic acid;
4-(acetylamino)-alpha-(3-mercaptopropyl)-benzenepropanoic acid;
3-(1-carboxy-4-mercaptobutoxy)-4-methoxy-benzoic acid;
4-(carboxymethyl)-alpha-(3-mercaptopropyl)-benzenepropanoic acid;
2-(1-carboxy-4-mercaptobutoxy)-benzoic acid;
4-(1-carboxy-4-mercaptobutoxy)-benzoic acid;
3-carboxy-2-chloro-alpha-(3-mercaptopropyl)-benzenepropanoic acid;
3-carboxy-4-chloro-alpha-(3-mercaptopropyl)-benzenepropanoic acid;
3-(1-carboxy-4-mercaptobutoxy)-4-chloro-benzoic acid;
3-(1-carboxy-4-mercaptobutoxy)-4-fluoro-benzoic acid;
5-carboxy-2-fluoro-alpha-(3-mercaptopropyl)-benzenepropanoic acid;
5-carboxy-alpha-(3-mercaptopropyl)-2-methoxy-benzenepropanoic acid;
4-carboxy-alpha-(3-mercaptopropyl)-1-naphthalenepropanoic acid;
2-carboxy-alpha-(3-mercaptopropyl)-benzenepropanoic acid;
4-carboxy-2,3,5,6-tetrafluoro-alpha-(3-mercaptopropyl)-benzenepropanoic acid;
5-mercapto-2-(phenylthio)-pentanoic acid;
3-[1-carboxy-4-mercaptobutyl)thio]-benzoic acid;
alpha-(3-mercaptopropyl)-2-naphthalenepropanoic acid;
2-chloro-alpha-(3-mercaptopropyl)-benzenepropanoic acid;
alpha-(3-mercaptopropyl)-3-[[(phenylmethyl)amino]carbonyl]-benzenepropanoic acid;
3-bromo-5-carboxy-alpha-(3-mercaptopropyl)-benzenepropanoic acid;
3-[[(carboxymethyl)amino]carbonyl]-alpha-(3-mercaptopropyl)-benzenepropanoic acid;
3-bromo-4-carboxy-alpha-(3-mercaptopropyl)-benzenepropanoic acid;
3-carboxy-alpha-(3-mercaptopropyl)-5-nitro-benzenepropanoic acid;
3-carboxy-5-(1,1-dimethylethyl)-alpha-(3-mercaptopropyl)-benzenepropanoic acid;
5-carboxy-alpha-(3-mercaptopropyl)-2-nitro-benzenepropanoic acid;
3′-(2-carboxy-5-mercaptopentyl)-[1,1′-biphenyl]-3-carboxylic acid;
2-bromo-5-carboxy-alpha-(3-mercaptopropyl)-benzenepropanoic acid;
(+)-3-carboxy-alpha-(3-mercaptopropyl)-benzenepropanoic acid;
(−)-3-carboxy-alpha-(3-mercaptopropyl)-benzenepropanoic acid;
5-(2-carboxy-5-mercaptopentyl)-[1,1′-biphenyl]-3-carboxylic acid;
2-(2-carboxy-5-mercaptopentyl)-[1,1′-biphenyl]-4-carboxylic acid;
6-(2-carboxy-5-mercaptopentyl)-[1,1′-biphenyl]-2-carboxylic acid;
4-(2-carboxy-5-mercaptopentyl)-[1,1′-biphenyl]-2-carboxylic acid;
3-carboxy-alpha-(3-mercaptopropyl)-5-methoxy-benzenepropanoic acid;
3′-(2-carboxy-5-mercaptopentyl)-[1,1′-biphenyl]-2-carboxylic acid;
3-(2-carboxyphenoxy)-alpha-(3-mercaptopropyl)-benzenepropanoic acid;
4-(2-carboxyphenoxy)-alpha-(3-mercaptopropyl)-benzenepropanoic acid;
3-carboxy-alpha-(3-mercaptobutyl)-benzenepropanoic acid;
4′-(2-carboxy-5-mercaptopropyl)-[1,1′-biphenyl]-2-carboxylic acid;
3-carboxy-alpha-(3-mercaptopropyl)-5-(phenylmethoxy)-benzenepropanoic acid;
alpha-(3-mercaptopropyl)-3-phenyl-benzenepropanoic acid;
3-carboxy-alpha-(3-mercaptopropyl)-5-phenoxy-benzenepropanoic acid;
3-carboxy-5-(1,1-dimethylethyl)-alpha-(3-mercaptobutyl)-benzenepropanoic acid;
3-(1,1-dimethylethyl)-alpha-(3-mercaptopropyl)-benzenepropanoic acid;
3-(1-carboxy-4-mercaptobutoxy)-5-(1,1-dimethylethyl)-benzoic acid;
3-[(1-carboxy-4-mercaptobutyl)thio-5-(1,1-dimethylethyl)-benzoic acid; and
3-[(1-carboxy-4-mercaptobutyl)amino]-5-(1,1-dimethylethyl)-benzoic acid.

### PMSA Ligands (G.1)

In various embodiments, the PSMA ligands represented by variable A in Structural Formulas A1, A2, A3, A11, A12, and A14 can be those described in U.S. Published Patent Application No. US2004/0014739, or the patents and applications to which it claims priority, U.S. Pat. No. 6,627,625 and U.S. Provisional Patent Application Nos. 60/149,115, 60/172,452, 60/176,570 and 60/194,534. The entire teachings of each of these documents are incorporated herein by reference. In some embodiments the PSMA ligand is optionally substituted clavulinic or a pharamceutically acceptable salt or solvate thereof. Suitable optional substituents are those described herein or more typically as described in the references in this paragraph.

### Excluded Compounds (F.1)

In some embodiments, the compounds of Structural Formula A1 and its dependent formulas A2-A14 are as described herein, provided that the compounds do not include compounds represented by structural formulas Fa-Fi:

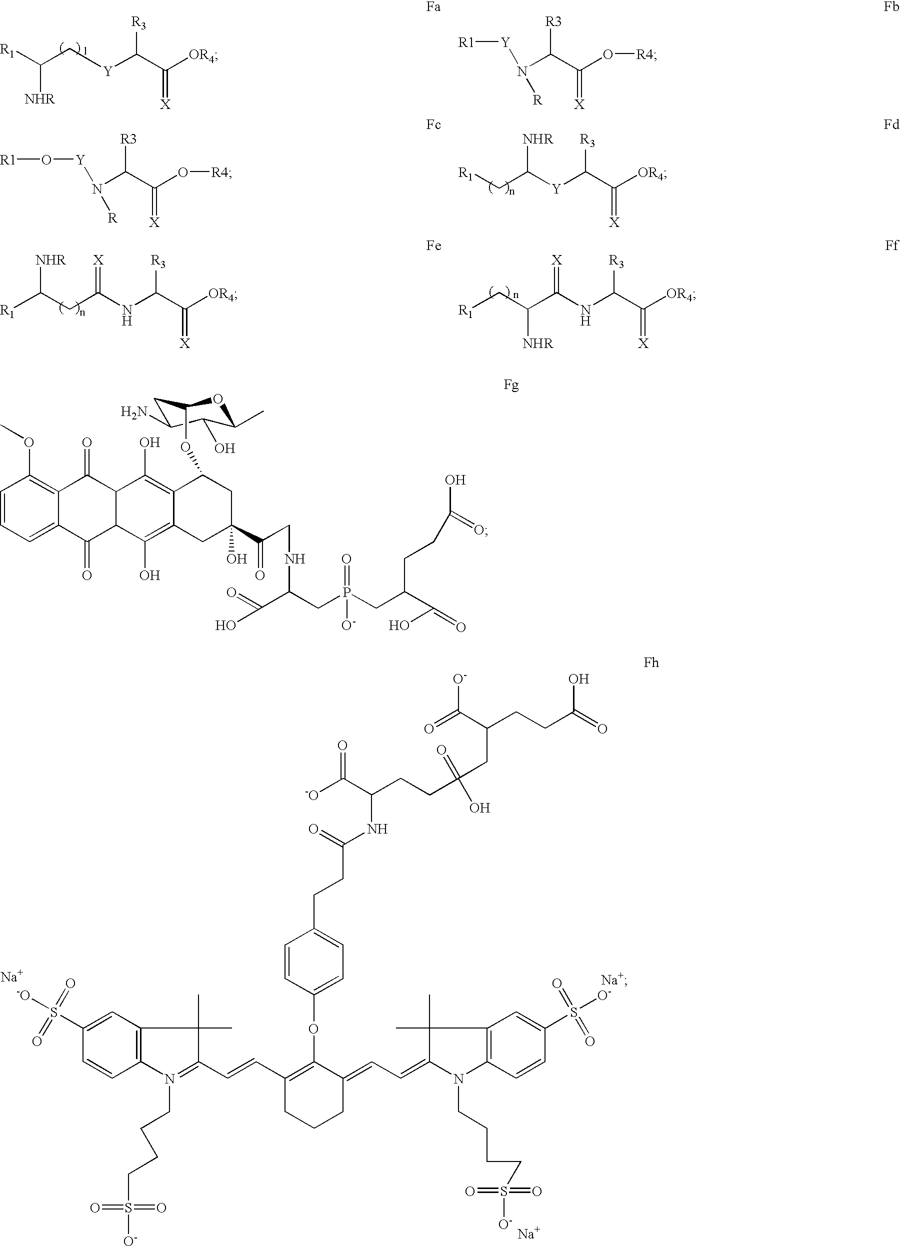

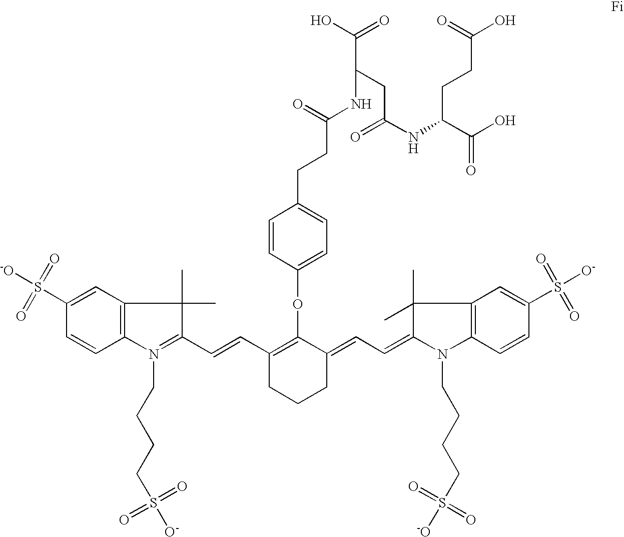

The variables in structural formulas Fa-Ff have the specific definitions given below in this section (F.1).

X represents O or S;

Y represents:

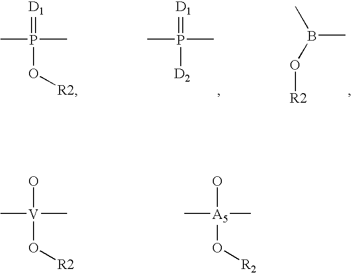

R represents a chelate ligand, a fluorescence tag, or a cytotoxic moiety. For example, the chelator ligan of R can be a chelator for a radiometal or a paramagnetic ion, e.g., a radionuclide useful for radiotherapy or imaging procedures such as a beta- or alpha-emitter for radio-therapeutic use, a gamma-emitter, positron-emitter, Auger electron-emitter, X-ray emitter or fluorescence-emitter, e.g., ^{99m}Tc (technium).

R1 and R3, independently for each occurrence, represents an alkyl, an alkenyl, a cycloalkyl, a cycloalkenyl, an aryl, —(CH₂)ₘ-aryl, -alkyl-CO₂R4, -alkenyl-CO₂R4, -cycloalkyl-CO₂R4, -cycloalkenyl-CO₂R4 or -aryl-CO₂R4;

R2 and R4, independently for each occurrence, represent hydrogen, a lower alkyl, or a pharmaceutically acceptable salt;

D₁ represents O or S;

D₂ represents N₃, SH₂, NH₂, or NO₂;

m is 1, 2, 3 or 4; and,

n is 0, 1, 2 or 3.

### Excluded Compounds (F.2)

In some embodiments, the compounds of Structural Formula A1 and its dependent formulas A2-A14 are as described herein, provided that the compounds do not include compounds represented by structural formula F2:

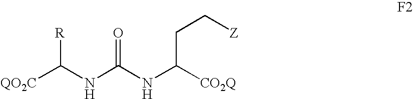

The variables in structural formula F2 have the specific definitions given below in this section (F.2).

R is selected from the group consisting of:
fluoroalkyl, typically having from 1 to 6 carbon atoms and about 1 and about 13 fluorine atoms;
aryl, typically having from 6 to about 12 carbon atoms and from 1 to 3 rings;
benzyl, typically having from 7 to 12 carbon atoms;
thiol and alkylthiol, typically having from 1 to about 6 carbon atoms, each of which is optionally substituted with an optionally substituted alkyl, typically having from 1 to about 6 carbon atoms;
optionally substituted alkenyl, typically having from 2 to about 6 carbon atoms;
optionally substituted alkynyl, typically having from 2 to about 6 carbon atoms;
optionally substituted aryl, typically having from 6 to about 12 carbon atoms in the ring and between about 1 and about 3 rings;
optionally substituted alkanoyl, typically having from 2 to about 6 carbon atoms;
or optionally substituted aralkyl, preferably having from 7 to about 12 carbon atoms;
optionally substituted alkoxy, typically having from 1 to about 6 carbon atoms;
optionally substituted aralkyloxy, typically having from 7 to about 12 carbon atoms;
or optionally substituted phenoxy, typically having from about 6 to about 12 carbon atoms and from about 1 to about 3 rings;

Q is hydrogen, optionally substituted alkyl, optionally substituted benzyl or optionally substituted phenyl; and
Z is Q or a tetrazole; or a pharmaceutically acceptable salt thereof.

### EXEMPLIFICATION

Described herein are new compounds, which can activate RNase L in PSMA expressing cells. 2-5A was targeted for delivery to PSMA by linking it with a ligand to PSMA. We modified ZJ24, a low molecular weight ligand for PSMA, with 2-5A through a 4-(N-maleimidomethyl)cyclohexane-1-amidate linkage to generate RBI 1033. The inhibitory activity of RBI 1033 to the folate hydrolase activity of PSMA was measured. The PSMA positive membrane preparations were incubated with different concentrations of ZJ24, RBI 1033 and RBI 1032 (2-5A with linker only) together with the substrate methotrexate diglutamate (MTXGlu₂) for 1 h. The amount of methotrexate and MTXGlu₂ were then determined by HPLC. Amazingly, it was found that compared to ZJ24 (IC₅₀=53.9 nM), RBI 1033 was more than 10 times more potent (IC₅₀=4.78 nM) as a folate hydrolase inhibitor, while 2-5A with linker alone showed an IC₅₀ at 1974 nM. Binding studies were performed by incubating membranes with different concentrations of drugs in the presence of 12 nM of ³H-ZJ24 for 30 min. RBI 1033 inhibited the binding of ³H-ZJ24 to the cell membrane with an IC₅₀ at 1.5 nM, compared to ZJ24 at 15.3 nM and 2-5A at 3481 nM. Therefore, RBI 1033 had a more than 10 times higher affinity to PSMA than ZJ24. These exciting results show that the new compound is more potent as a folate hydrolase inhibitor and has higher binding affinity to PSMA compared to the parent drug ZJ24. These studies indicate that RBI 1033 has excellent binding properties to PSMA, which can enable it to serve as a vector for imaging or therapy.

### Example 1

### Synthesis of PSMA Ligand (6) and S-Methylated Analog ZJ24

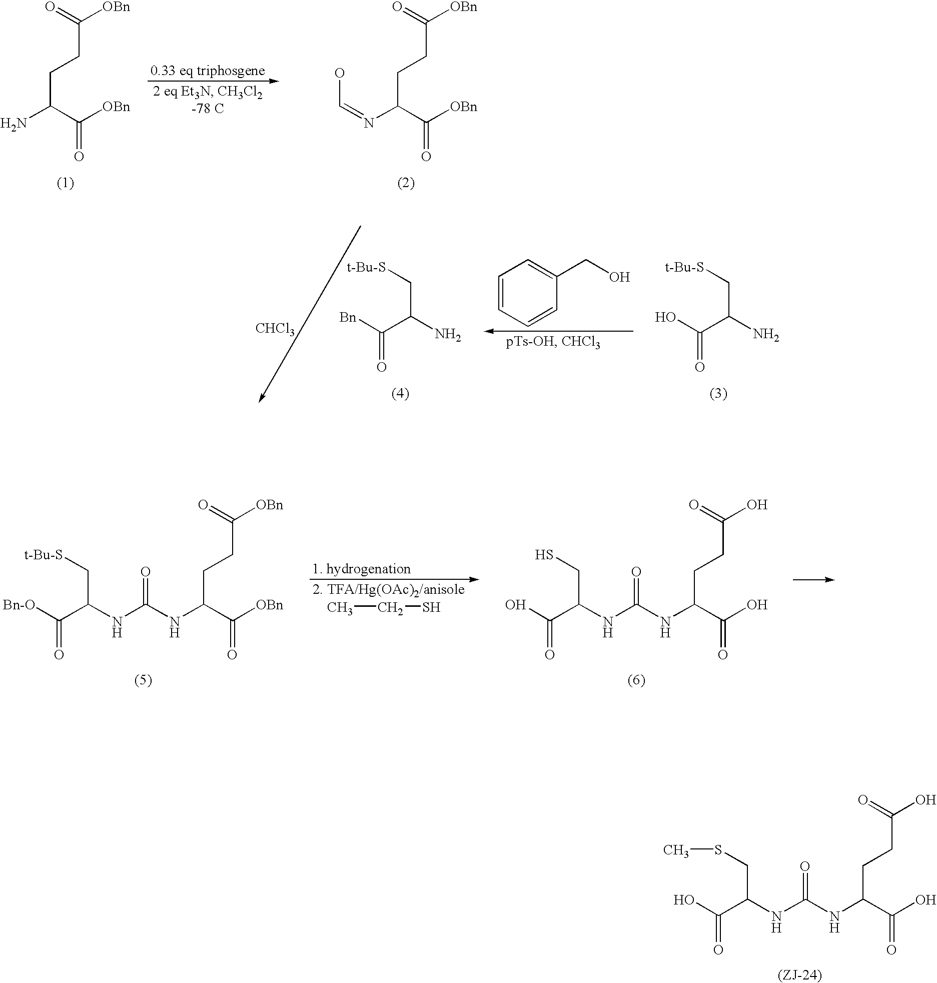

Synthesis of the asymmetric uryl dipeptide parent PSMA ligand (6) (corresponding to the PSMA ligand represented by variable A in Structural Formula A1) was accomplished through direct chemical addition of a carbonyl chloride to dibenzyl esterified glutamate (D-Glu(OBn)) (Advanced Chemtech, Louisville, Ky.) using triphosgene to form the corresponding isocyanate (2). This was followed by direct addition of a benzyl esterified cysteine with tert-butyl protection for the thiol (L-Cys(tBu)OBn) (Advanced Chemtech) and slowly warming to room temperature. Debenzylation was achieved through catalytic hydrogenation with Pearlman's catalyst (20% PdOH on carbon). The tert-Butyl group from cysteine was cleaved by treatment with TFA/Hg(OAc)₂/anisole followed by dihydrogen sulfide.

The last step shows methylation of the SH group to give compound ZJ24, the S-methylated analog of parent PSMA ligand (6). To a solution of PSMA ligand (6) (Cys-C(O)-Glu (1.7 mg, 5.84 umol) in DMF (0.10 mL) was added 0.10 mL DMF saturated with NH₃ (suspension). H₂O (50 μl) was then added and the reaction mixture became a clear solution. At 45° C., CH₃I (3.5 ul, 56.2 umol, 9.6 eq.) was added. Five minutes later, the reaction mixture was concentrated and purified via high pressure liquid chromatography (HPLC) to give product ZJ24 See Pomper, M. G., Musachio, J. L., Zhang, J., Scheffel, U., Zhou, Y., Hilton, J., Maini, A., Dannals, R. F., Wong, D. F., and Kozikowski, A. P. (2002). “11C-MCG: synthesis, uptake selectivity, and primate PET of a probe for glutamate carboxypeptidase II (NAALADase).” Mol. Imaging, 1, 96-101, the entire teachings of which are incorporated herein by reference.

This methylation can be employed to introduce a radiolabel into the ligand without compromising its activity. For example, by employing a ³H (tritium) radiolabled isotopologue of any standard methyltion reagent (e.g., [³H]MeI), a ³H (tritium) radiolabled analog of compound ZJ24 can be synthesized, ³H—(S)-2-[3-((R)-1-carboxy-2-methyl-sulfanyl-ethyl)-ureido]-pentanedioic acid or [³H]MeCys-C(O)-Glu.

### Example 2

### Preparation of Disclosed Compounds

2-5A is a small molecule drug that activates RNase L, an ubiquitous intracellular enzyme in man, which once activated can degrade viral and cellular RNA leading to apoptosis of a cell. Certain disclosed compounds (termed disclosed 2-5A ligands, e.g, compound (RBI 1033) below) can be synthesized from a 2-5A trinucleotide precursor (RBI 1024), an aliphatic linker precursor (e.g. succinimidyl-4-(N-maleimidomethyl)cyclohexane-1-carboxylate, or SMCC (25)) and a PSMA ligand (6) (precursors corresponding to CB, L, and A, respectively, in Structural Formula A1) by post-synthesis conjugation or by stepwise solid-phase synthesis of the complete conjugate:

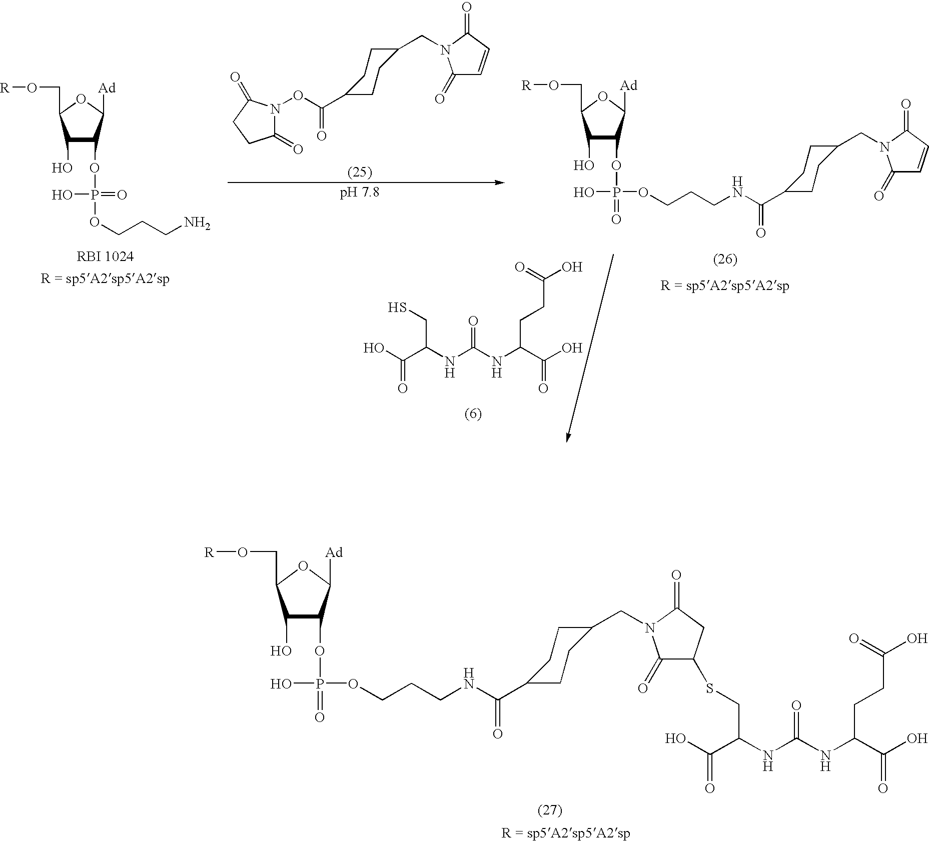

Note that phosphodiester bonds of natural 2-5A can be replaced with phosphorothioate linkages to increase its stability against enzymatic degradation. Post-synthesis conjugation can allow the precursors to be easily accessible and the final product can be separated more easily from the starting materials. The compound can also be synthesized via stepwise solid-phase synthesis.

### Example 2.1

### Post Synthesis Conjugation

Functional groups can be introduced at the 2′-end of the 2-5A trinucleotide moiety since a 5′-phosphate or 5′-phosphorothioate can be required for the activation of RNase L. 2′/3′-functional groups can be introduced into oligonucleotides by starting the synthesis on a modified support bearing already the functional group in a protected form to make it compatible with standard oligonucleotide synthesis.

The amino functionalized 2-5A analog (RBI 1024) was therefore prepared using a phthalimidyl modifier (Glen Research, Sterling, Va.). A thiol group can be introduced using a commercially available modifier for introducing 2′ thiols and subsequent reduction with dithiothreitol (DTT, Glen Research).

The 2-5A moiety was coupled with PSMA ligand 6 (precursors corresponding to CB and A, respectively, in Structural Formula A1) as shown in the above scheme by conjugation with the bifunctional linker SMCC. This approach can represent a simple and easy way to generate a peptide-oligonucleotide conjugate in high yields. The 3′-amino derivatized 2-5A compound was converted to the 3′-maleimide derivative 26 by coupling with the hetero-bifunctional linker sulfosuccinimidyl-4-(N-maleimidomethyl)-1-carboxylate (SMCC) 25, (Pierce, Wis.). After HPLC purification 26 was coupled to the ligand 6 in 0.1 M potassium phosphate (pH 7.8) for 3 hours. Excess peptide was removed using a NAP 5 gel filtration column and the final product was dried in vacuo. The final product was purified by HPLC and its mass confirmed by mass spectrometry using MALDI.

Alternatively, the ligand 6 can be conjugated to 3′-SH functionalized 2-5A analog via disulfide cross-linkage. Linking a SH-functionalized oligonucleotide to a cysteine containing peptide via a disulfide cross-linkage is well known in the art. Moreover, the yield can be improved by employing a modified method shown in Antopolsky, M., Azhayeva, E., Tengvall, U., Auriola, S., Jaaskelainen, I., Ronkko, S., Honkakoski, P., Urtti, A., Lonnberg, H., and Azhayev, A. (1999). “Peptide-oligonucleotide phosphorothioate conjugates with membrane translocation and nuclear localization properties.” Bioconjug. Chem., 10, 598-606, the entire teachings of which are incorporated herein by reference.

### Example 2.2

### Stepwise Solid-Phase Synthesis of Disclosed Compounds

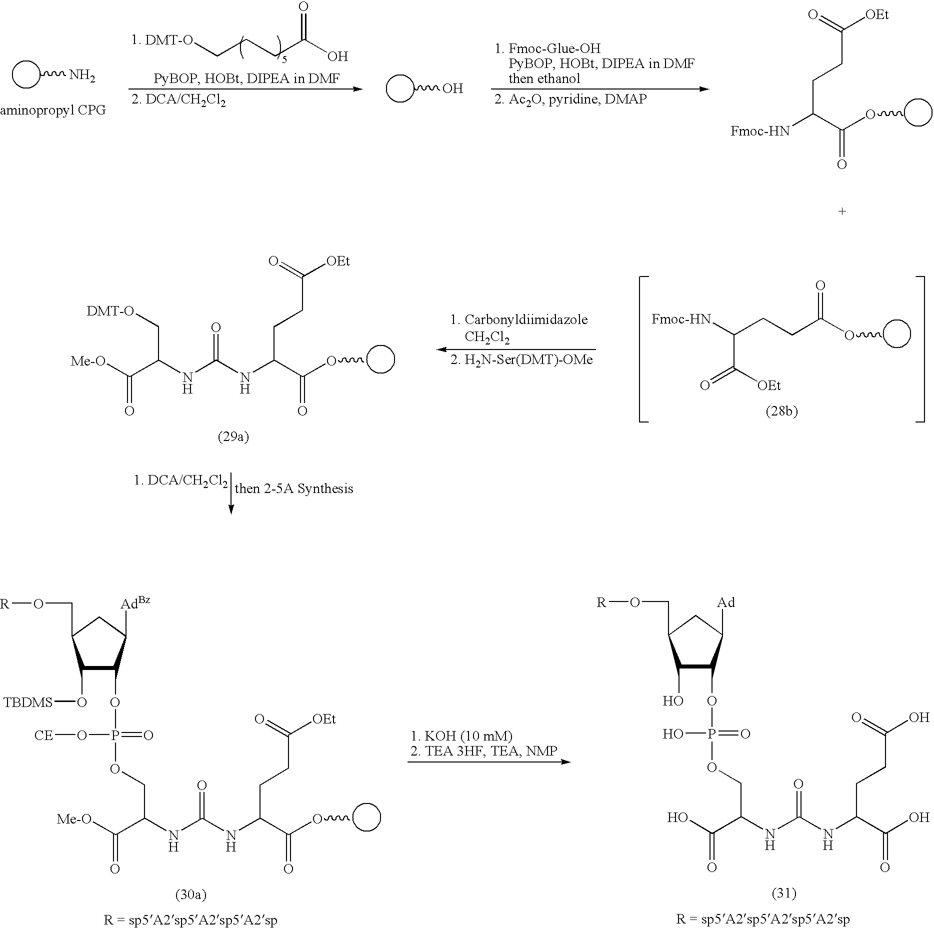

Stepwise solid-phase synthesis is used to demonstrate synthesis of two different disclosed compounds. In the first approach a 2-5A trinucleotide is linked to the PSMA ligand peptide by an amide bond. In the second approach, a 2-5A trinucleotide is linked to the PSMA ligand peptide through a phosphodiester bond. The stepwise solid-phase approach can have the advantage that no postsynthetic conjugation steps are required, avoiding intermediate purification steps and can also result in higher overall yields. However, because standard oligonucleotide and peptide synthesis are typically not compatible with each other, amino acid protecting groups are employed that can be compatible with oligonucleotide synthesis. Appropriately protected amino acid monomers are made from commercially available precursors.

### Solid Support Synthesis of 2-5A Lys-C(O)-Glu Ligand

Solid supports useful for 2-5A ligands are prepared by coupling 12-hydroxy lauric acid linker to aminopropyl CPG. 12-hydroxy lauric acid as well as γ-hydroxybutyric acid linker (used at a later step of the synthesis) are easily accessible by tritylation of the appropriate acids. Fmoc-Glu-OH (Advanced Chemtech) is coupled to the support under regular peptide synthesis conditions. The remaining free carboxyl groups are esterificated by adding ethanol to the reaction mixture. After the capping step, a carbonyl group is introduced using carbodiimidazole. The resulting activated urea is reacted directly with an appropriately protected lysine. Detritylation, subsequent peptide coupling with γ-hydroxybutyric acid linker, combined with another capping and detritylation step generates a free hydroxyl group useful as the starting point for conventional solid-phase 2-5A synthesis.

### Solid Support Synthesis of 2-5A Ser-C(O)-Glu Ligand

This strategy is based on an approach for making 3′,5′-dipeptidyl oligonucleotides recently published by Schwope et al. The solid support is functionalized as described above. This time the activated urea is reacted with an appropriate protected serine. The serine's free hydroxyl group is used as starting point for conventional solid-phase 2-5A oligonucleotide synthesis as shown above.

### Example 3

### PSMA Binding and Activity Assay Shows Disclosed Compounds have Improved Binding Compared to Parent PSMA Ligand

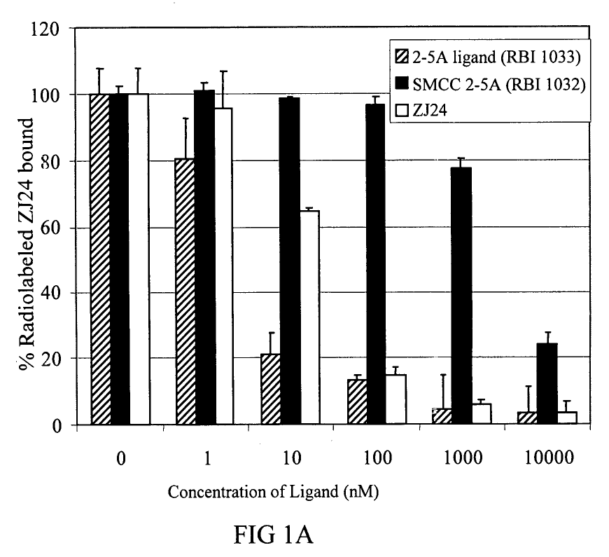

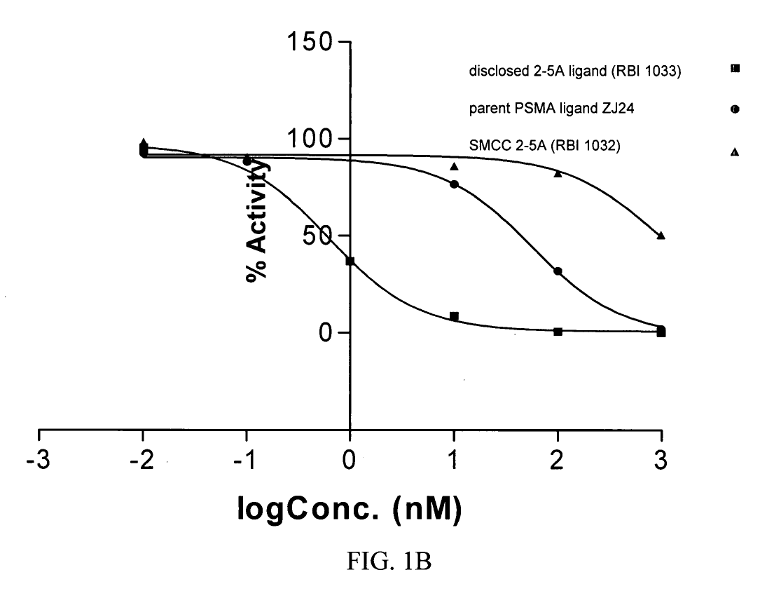

FIG. 1A is a bar graph showing percentage of radiolabeled compound bound versus concentration where the disclosed 2-5A ligand binds to PSMA at lower concentrations (EC₅₀=1.5 nM) compared to compound (ZJ24) (the S-methylated analog of parent PSMA ligand (6)) (EC₅₀=15.3 nM) or the intermediate 2-5A-SMCC oligonucleotide-linker (26) (RBI 1032) (which can be used to probe for nonspecific binding). Binding and activity of the disclosed 2-5A ligand towards the active dimeric form of soluble recombinant hPSMA was compared with methylated parent ligand Cys-C(O)-Glu ZJ24. The binding assay examines the ability of a novel compound to compete for binding with radio-labeled ZJ24. Different final concentrations of the inhibitor can be incubated in the presence of ZJ24 and the concentration required to inhibit 50% of binding is determined (the EC₅₀). Briefly, 0.5 μg of recombinant PSMA was incubated with the ligand to be tested at different concentrations and 12 nM of ³H-ZJ24 in a total volume of 100 μL for 30 min at 37° C. The mixture was centrifuged through a 50 kDa cut-off membrane. Finally, 4 mL of biodegradable counting cocktail Bio-Safe II was added, and radioactivity was counted.

FIG. 1B is a plot of activity versus log(concentration) for the three compounds, where the disclosed 2-5A ligand inhibits PSMA at lower concentrations (EC₅₀=0.62 nM) compared to compound (ZJ24) (the S-methylated analog of parent PSMA ligand (6)) (EC₅₀=56.7 nM) or the intermediate 2-5A-SMCC oligonucleotide-linker (26) (RBI 1032). The activity assay determines the ability of hPSMA to cleave/hydrolyze a polyglutamte substrate under the presence of a ligand at different concentrations. Briefly, recombinant PSMA (5 ng) was incubated with ZJ24 or disclosed 2-5A ligand. After 30 min the polyglutamate substrate MTXGlu2 (5 nMol) was added and the mixture was incubated at 37° C. for 1 h. The reaction was stopped by the addition of 100 μL of 50 mM Na₂HPO₄. The amount of MTX formed was analyzed using a Thermo Hypersil PRISM RP column (50×4.6 mm, flow rate 1.0 mL/min) at 313 nm.

The disclosed 2-5A ligand is superior in binding and inhibiting PSMA compared with the methylated parent PSMA ligand ZJ24 as shown in FIGS. 1A and 1B. The EC₅₀ for binding was found to be 1.5 nM for the novel 2-5A ligand versus 15.3 nM for ZJ-24, while the inhibitory activity of the disclosed compound was 0.62 nM in comparison to 56.7 nM for the parent PSMA ligand. To verify that this increased activity is not due to non-specific binding of the 2-5A or SMCC part of the molecule, the binding of the 2-5A-SMCC fragment to PSMA was also measured. However, it was found that the −5A-SMCC fragment alone bound only weakly to PSMA with an EC₅₀ of 3.5 μM (FIG. 1A).

### Example 4

### Targeting Rnase L-Activator Drugs to Prostate Cancer Cells Through Prostate Specific Membrane Antigen (PSMA)

In normal prostate epithelia, PSMA can be expressed primarily as a cytoplasmic protein termed PSM′. In prostate carcinomas, however, differential mRNA splicing can lead to expression of PSMA as a 100-kDa type II transmembrane glycoprotein possessing a 19-aa cytoplasmic fragment, a single 24-aa membrane-spanning domain, and a 707-aa extracellular region. PSMA is a cell-surface membrane protein that is not secreted, in contrast to other well-known, unrelated prostate-restricted molecules such as prostate specific antigen (PSA) and prostatic acid phosphatase (PAP) that are secretory proteins, thereby making PSMA a suitable target for prostate cancer therapy. PSMA has an internalization signal MXXXL that is responsible for the internalization of the protein on the cell surface into endosomal and lysosomal compartments. Moreover, because PSMA can be subject to receptor-mediated endocytosis, it can be expected that RNase L-activator/ligand conjugates can enter prostate cancer cells and endothelial cells of tumor neovasculature.

By chemically linking a novel small molecule activator of RNase L (e.g., a 2-5A oligonucleotide moiety such as 2-5A oligonucleotide RBI 1024) to a PSMA ligand moiety, a two-part drug is generated that can target prostate cancer cells and can be internalized in prostate cancer cells.

In order for the candidate drug to be effective, it can bind to both PSMA and to RNase L. In addition, the drug candidate can activate RNase L. To determine these properties, isolated

PSMA and RNase L proteins are used. Further, cell uptake and RNase L activation assays in intact prostate cancer cells can be conducted as described below.

### Example 4.1

### Binding of PSMA

PSMA can exist in dimeric and monomeric form. Recombinant protein of the extracellular domain of PSMA can also exist in readily inter-convertible dimer-monomer forms. PSMA can be expressed as non-covalent homodimer on the surface of prostate cancer cells. It is yet to be characterized whether the dimer or monomer undergo internalization, or what induces dimer formation or causes dissociation to the monomer form. The difference can be important because the dimer typically has enzymatic activity and the monomer typically does not.

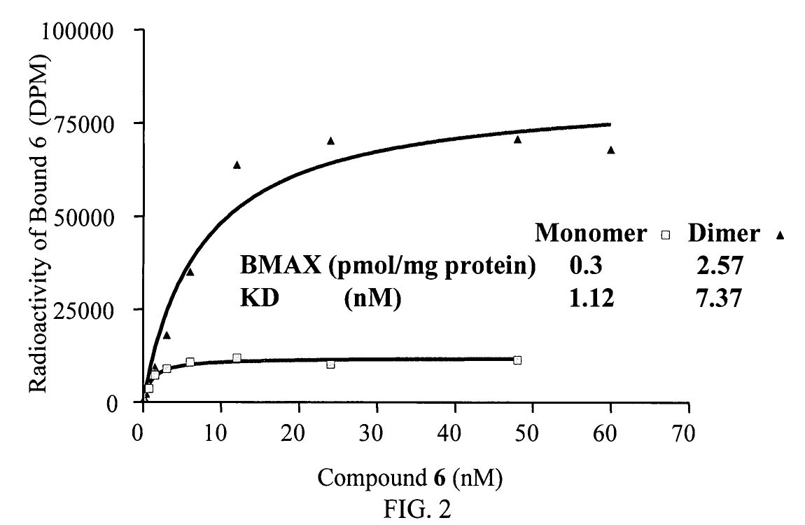

FIG. 2 is a graph showing binding to the monomeric and active dimeric form of soluble recombinant hPSMA of a ³H (tritium) radiolabled (ZJ24), ³H—(S)-2-[3-((R)-1-carboxy-2-methyl-sulfanyl-ethyl)-ureido]-pentanedioic acid or [³H]MeCys-C(O)-Glu Parent PMSA ligand (6) maintains its specificity for PSMA by binding to recombinant hPSMA with a K_{d} of 2 nM based on Scatchard analysis. The binding assay was performed in 50 mM Tris buffer (pH 7.5). Briefly, 0.5 μg of recombinant PSMA was incubated with ³H-ZJ24 in a total volume of 100 μL for 30 min at 37° C. The mixture was centrifuged through a 50K cutoff membrane. Then 50 μL of filtrate was added to 4 mL of cocktail and the radioactivity measured. Also, an ¹¹C-labeled analog of ZJ24 was found to be quite stable in animal models (mouse and primate). After 60 min less than 10% of the compound found in kidney and plasma was degraded.

This can be adapted to determine PSMA binding of the disclosed compounds. 0.5 μg PSMA is incubated with a radiolabeled analog of a disclosed compound in a total volume of 100 μL for 30 min at 37° C. The mixtures are centrifuged through a Centricon cartridge Biomax Membrane with a 50,000 Da exclusion limit (Millipore cat. No. UFC₂BQK). Then 50 μl of the filtrate, containing the unbound ligand, is added to 4 ml scintillation cocktail mixture and the radioactivity is measured. The binding affinity (K_{d}) of the 2-5A-PSMA ligand or agonist-PSM ligand conjugates is determined with recombinant dimer PSMA in competition binding assays with the radiolabeled ligand.

### Example 4.2

### Binding and Activation Assay of RNase L

Binding of the disclosed 2-5A-PSMA ligands to RNase L is performed by the 2-5A binding competition assay as described above. Briefly, recombinant purified human RNase L is incubated with a radiolabeled 2-5A analog [p(A2′p)₂(br⁸A2′p)₂A3′[³²P]pCp] in the absence or presence of different concentrations of the 2-5A-PSMA ligand conjugate. Incubation is for 1 h on ice, followed by an additional 1 h on ice under UV (308 nm) light. Proteins are separated by SDS/PAGE and the amount of radiolabeled 2-5A probed bound to RNase L is measured by phosphorimage analysis of the dried gels. Activation of RNase L by the 2-5A-PSMA ligand conjugates is determined the FRET assay described above. Briefly, recombinant RNase L and the FRET RNA probe are incubated at 22° C. for 100 min in the absence or presence of different concentrations (0.1 nM to 10 μM) of the 2-5A-PSMA ligand conjugates. Cleavage of the FRET RNA probe is measured in a Wallace Victor2 model 1240 fluorospectrometer at 485 nm excitation, 535 nm emission.

### Example 4.3

### Determining if the 2-5A-Ligand Conjugates can Enter Prostate Cancer Cells and Activate RNase L

PC3 cells lack PSMA, while a PC3 derivative cell line, PC3_{PIP} cells, express PSMA from a cDNA expressing PSMA. To determine if PSMA ligand conjugated with 2-5A can penetrate cells expressing PSMA, these compounds are added at concentrations ranging from 0.1 to 25 μM to media of PC3_{PIP} cells. RNase L activators, either chemically conjugated to the PSMA ligand or, as a control, added to PSMA ligand without conjugation, are incubated with cells for 3 to 8 h at 37° C. Total RNA is isolated and analyzed for RNase L-specific cleavages of 18S and 28S rRNA in RNA chips as described (Section C1, FIG. 4A). Specific rRNA cleavages can be evidence that the RNase L-activator enters the cells. To determine if the 2-5A-PSMA ligand conjugates can cause apoptosis, PC3_{PIP} cells are incubated for 24 h with the compound followed by FACS TUNEL assays.

### Example 4.4

### Binding of 2-5A Trinucleotide RBI 1033 to LNCaP and PC-3 Cells Expressing PSMA (PC3_{Pip}) But not to Cells not Expressing PSMA (PC3_{flu})

To obtain further evidence for selectivity, the parental PC3 cells are labeled by stable expression GFP cDNA. Subsequently, equal numbers of the PC3 and PC3_{PIP} cells are mixed and seeded in 24-well plates. On the following day, the PSMA ligand conjugated drugs are added to the mixed cultures at different concentrations (nM to μM). Relative numbers of GFP-expressing and unlabeled cells are determined by FACS analysis. If PSMA-targeting and RNase L-activation is successful, an elimination of the cells lacking GFP (and PSMA) is observed. RNase L activator-PSMA ligand conjugates which are demonstrated to both enter PC3_{PIP} cells unassisted and which can preferentially cause apoptosis in PSMA expression PC3 cells are further tested in the human prostate cancer xenografts in nude mice.

To this end, drug candidates will consist of an activator of RNase L covalently bound to PSMA-ligand. PSMA-ligand will target this drug to prostate carcinoma cells and tumor vasculature where activated RNase L will efficiently degrade cellular mRNA and induce apoptosis. PSMA-ligand drugs will be tested against PC3_{PIP} cells, stably transfected to express PSMA (6), in the subcutaneous and in the orthotopic model. The same dosing scheme will be followed as above. There will be three (3) experimental groups (PBS, one-half MTD, MTD) and ten (10) mice per group. In addition, since the PSMA-ligand drugs are predicted to ablate tumor vasculature, dermal angiogenesis studies will be conducted with five (5) such compounds. There are a number of angiogenesis assays. We will use the in vitro assay described by Woltering and will use human tissues grown in fibrin clots as PSMA does not appear to be expressed in non-human endothelial cells during angiogenesis.

### Example 4.5

### Assess Anti-Tumor Activity of Activators of RNase L

The PSMA-ligand drugs are analyzed for anti-tumor activity in a subcutaneous prostate carcinoma model in athymic nude mice. For example, there are ten (10) mice per experimental group in the subcutaneous model. Tumor volume is measured and mice are weighed three times a week. Mice are observed for euthanasia criteria, including presence of large tumors (>10% of body weight), weight loss (>10% of body weight), lethargy, general inactivity, difficulty ambulating, huddled posture, vocalization, or obvious morbidity.

The most effective drug candidates that emerge from the subcutaneous tumor model screen can advance to the orthotopic (intra-prostatic) tumor model. For example, there are ten (10) mice per experimental group in the orthotopic model. Five (5) PSMA-ligand drugs are be evaluated in the murine dermis (angiogenesis) models. For example, there are ten (10) mice per experimental group in the angiogenesis model.

### Example 5

### Assess Toxicity of Novel Ligands to Non-PSMA Expressing Cells

The disclosed 2-5A ligands can be tested for toxicity to non-cancerous and other non-PSMA expressing cells. Tetrazolium Conversion (MTS) assays are used to study the cytotoxicity of the new compounds in PSMA expressing (PC-3 pip) and PSMA negative PC3 cells as well as primary normal prostate epithelial (PrEC) cells. Cells are seeded in 96-well microtitre plates. Drugs are added after series dilution and exposed to cells for 72 h, after which MTS solution is added and the cells are incubated for 4 h at 37° C. Absorbance is measured, e.g., at 490 nm using a microtitre plate reader.

### Example 6

### Synthesis of Disclosed Compound Having a Fluorescent-Labeling Agent

Synthesis of a fluorescent derivative of a 2-5A ligand is conducted analogously to its parent compound, with the exception that a commercially available fluorophore (e.g., fluorescein amidite (Glen Research, VA) is coupled to the solid support prior to the first 2-5A phosphoramidite addition.

### Example 7

### Synthesis of Disclosed Compound Having a Fluorescent-Labeling Agent

Analogs of 2-5A ligand (27) are synthesized with shortened 2-5A moieties and are tested for binding and inhibitory activity using synthesis and biological assay procedures as described above. The position indicated by R in (27) can be replaced with H, phosphorothioate, labeling agents, drugs, and the like.

For example, one or more adenosine moieties in 2-5A ligand (27) are replaced with 5-iodo-2′-deoxyuridine (IUdR) to incorporate the radioisotope I-124 into the ligand as a labeling agent for in vitro and in vivo imaging studies, for example, the position indicated by R in (27) can be replaced with I-124 IUdR. Such a label can be added to the 5′-end of any adenosine modified ligand.

For example, a labled ligand is characterized for its in vitro and in vivo binding and preclinical optimization for in vivo imaging using PSMA expressing human prostate cancer cell lines, e.g., LNCaP, C₄₋₂, MDA-Pca 2b, and CRW22 as PSMA positive tumor targets and non PSMA expressing PC3 and DU145 as negative controls. The binding of a ligand to cell membrane or live cells is accomplished by incubation of the membrane preparations or cells with radiolabeled or radiolabeled +cold inhibitor, then centrifuging bound cells to determine binding constants.

Biodistribution studies are conducted by procedures previously described for PSMA targeting monoclonal antibodies (Smith-Jones, et al. (2003). “Radiolabeled monoclonal antibodies specific to the extracellular domain of prostate-specific membrane antigen: preclinical studies in nude mice bearing LNCaP human prostate tumor.” J. Nucl. Med., 44, 610-7, the entire teachings of which are incorporated herein by reference). PSMA positive and PSMA negative cells (0.1 mL cell suspension containing 5×10⁶ cells) are implanted subcutaneously in the flanks of 8-10 week old Nu/Nu BALB/c mice (8 mice/group). When tumors reach a size of about 100 mm³, mice receive an injection of radiolabeled ligand into the tail vein either alone or together with a 100 fold excess of unlabeled ligand to block specific binding. Animals are sacrificed by CO₂ inhalation after 2, 8, 24 and 48 hours of injection and the radioactivity in brain, blood, heart, lung, kidney, liver, prostate and tumor is determined. Also, a PET scanner, e.g., the microPET R4 scanner (Concorde MicroSystems) from the Small Animal Imaging Center at Case Western Reserve University (http://cancer.cwru.edu/shared/shared_con.html) is used for imaging repeat scans and for biodistribution studies of 124, labeled ligand. 1241 has an energy spectrum that is compatible for PET imaging so it can be possible to image the mice in planar images.

### Example 8

### On Prostate Cancer Cells and Tumor Neovasculature Endothelial Cells with 2-5A-ligand Conjugates

Delivery and stability can be major challenges for oligonucleotide-based drug development. While small molecule cancer drugs can be more stable, their selectivity for cancer cells can be less than ideal which can make such drugs toxic to the human body. PSMA is almost exclusively expressed on prostate cancer cells and tumor vascular endothelium. Targeting PSMA can thus specifically target prostate cancer or solid tumors that express PSMA in their neovasculature.

RNase L activation by 2-5A analogs has been shown to lead to apoptosis and therefore to the killing of tumor cells. Also, a 2-5A antisense compound targeting telomerase was found to be active in vivo and in vitro. However, so far delivery to the target organ has not been addressed.

The disclosed compounds, by chemically linking a 2-5A moiety to a PSMA ligand moiety give 2-5A ligands with molecular weights <1500 Da, which can be recognized by PSMA and can be subsequently internalized into a prostate cancer cell. In comparison to monoclonal antibody strategies currently in clinical trials for imaging and therapy these molecules are very small and can diffuse into the tumor area more quickly.

The 2′-terminal hydroxyl group in 2-5A was chosen as the mode of linkage to the ligand to continue the pattern of 2′-phosphorylation in its backbone. A free 5′-phosphorothioate was included since a 5′-phosphoryl can lead to activation of RNase L. The substitution from an oxygen to a sulfur (phosphate to phosphorothioat) can lead to increased resistance towards phosphatases. Also, this “tailing” of 2-5A can provide resistance to degradation by 2′,5′-phosphodiesterases. Internucleotide phosphorothioate linkages were chosen to further increase resistance against degradation by 2′,5′-phosphodiesterases.

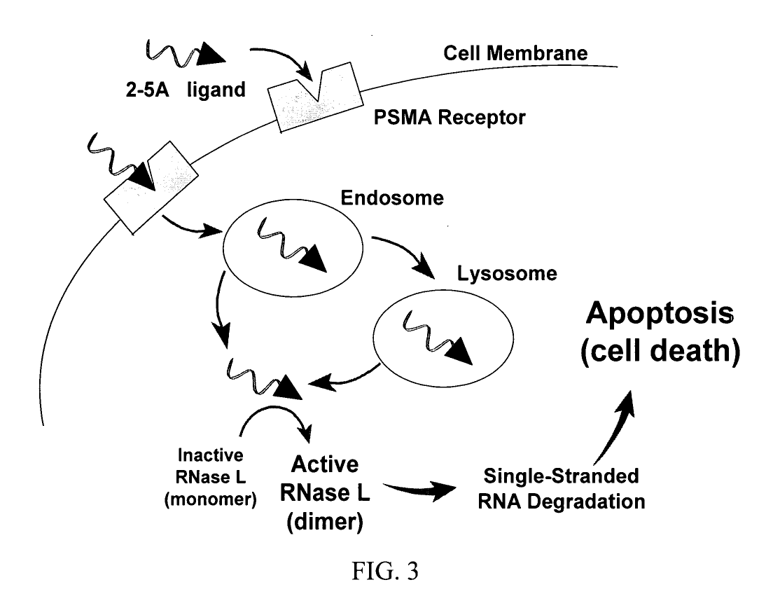

FIG. 3 shows PSMA receptor-mediated internalization of 2-5A ligands. The endocytic pathway which includes internalization of the receptor-ligand complex via clathrin-coated pits and accumulation in the endosomes. The receptor-ligand complex can dissociate in the endosomes and the dissociated molecules can be either recycled back to the cell surface or are targeted to lysosomes for degradation. However, despite this apparent problem, receptor mediated endocytosis has been successfully used for the delivery of a portfolio of drugs including oligonucleotide type drugs. Without wishing to be bound by theory, it is believed that chemotherapeutic agents including oligonucleotides are able to leak through the endosomal and lysosomal membranes. It is possible that the ligand part of the conjugate will be digested if a lysosome is formed during the internalization cycle.

The entire teachings of each reference cited herein is incorporated by reference.

While this invention has been particularly shown and described with references to preferred embodiments thereof, it will be understood by those skilled in the art that various changes in form and details may be made therein without departing from the scope of the invention encompassed by the appended claims.

1-20. (canceled)

21. A compound represented by the following structural formula:
C—B-L-A
or a pharmaceutically acceptable salt or solvate thereof, wherein:
A is a prostate specific membrane antigen (PSMA) ligand;
L is an optionally substituted aliphatic or heteroaliphatic linking group;
B comprises at least two optionally substituted moieties selected from the group consisting of a sugar, a charged group, an aryl ring, and a heteroaryl ring; wherein B optionally comprises a drug or a labeling agent; and
C is H, a drug, or a labeling agent,
wherein CB together comprises the drug or the labeling agent.

22-23. (canceled)

24. The compound of claim 21 wherein the compound is represented by the following structural formula:
C-(ZX⁴X⁵)ₛ-L-A
wherein:
X⁴ is a bond, —NR^{a}—, —O—, —S—, —CR^{a}R^{b}—, —CR^{b}(OR^{a})—, —CR^{b}(SR^{a})—, —C(O)—, —C(S)—, —C(═CR^{a}R^{b})—, —C(═NR^{a})—, —C(═NOR^{a})—, —C(═NNR^{a})—, —S(O)—, —(SO₂)—, —S(O)(R^{a})—, —S(O)(OR^{a})—, —(PO₂)—, —P(O)(R^{a})—, —P(O)(OR^{a})—, —OP(O)(R^{a})—, —OP(O)(OR^{a})—, —P(S)(R^{a})—, —P(S)(OR^{a})—, —OP(S)(R^{a})—, or —OP(S)(OR^{a})—;
L is an optionally substituted aliphatic or heteroaliphatic linking group;
s is an integer from 1 to 6, wherein the variables in each (ZX⁴X⁵) are independently selected;
each Z is independently an optionally substituted aryl, heteroaryl, cycloaliphatic, or non-aromatic heterocyclic group, provided that at least one Z is an aryl or heteroaryl group or is substituted with an aryl or heteroaryl group; and
X⁵ is a bond or methylene,
wherein R^{a} and R^{b} are each independently —H or an optionally substituted aliphatic, optionally substituted cycloaliphatic, optionally substituted heterocyclic, optionally substituted benzyl, optionally substituted aryl, or optionally substituted heteroaryl.

25. The compound of claim 24, wherein optional substituents are independently selected from the group consisting of —F, —Cl, —Br, —I, —CN, —NO₂, —OR^{a}, —C(O)R^{a}, —OC(O)R^{a}, —C(O)OR^{a}, —SR^{a}, —C(S)R^{a}, —OC(S)R^{a}, —C(S)OR^{a}, —C(O)SR^{a}, —C(S)SR^{a}, S(O)R^{a}, —SO₂R^{a}, —SO₃R^{a}, —POR^{a}R^{b}, PO₂R^{a}R^{b}, PO₃R^{a}R^{b}, —PO₄R^{a}R^{b}, P(S)R^{a}R^{b}, P(S)OR^{a}R^{b}, —P(S)O₂R^{a}R^{b}, —P(S)O₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{a}, —C(O)NR^{a}CN, —SO₂N(R^{a}R^{b}), —SO₂N(R^{a}R^{b}), —NR^{c}C(O)R^{a}, —NR^{c}C(O)OR^{a}, —NR^{c}C(O)N(R^{a}R^{b}), —C(NRC)—N(R^{a}R^{b}), —NR^{d}—C(NR^{c})—N(R^{a}R^{b}), —NR^{a}N(R^{a}R^{b}), —CRC═CR^{a}R^{b}, —C≡CR^{a}, ═O, ═S, ═CR^{a}R^{b}, ═NR^{a}, ═NOR^{a}, ═NNR^{a}, optionally substituted alkyl, optionally substituted cycloalkyl, optionally substituted aliphatic, optionally substituted cycloaliphatic, optionally substituted heterocyclic, optionally substituted benzyl, optionally substituted aryl, and optionally substituted heteroaryl; wherein R^{a}—R^{d} are each independently —H or an optionally substituted aliphatic, optionally substituted cycloaliphatic, optionally substituted heterocyclic, optionally substituted benzyl, optionally substituted aryl, or optionally substituted heteroaryl, or, —N(R^{a}R^{b}), taken together, is an optionally substituted heterocyclic group.

26. The compound of claim 25, wherein C-(ZX⁴X⁵)s- comprises an optionally substituted nucleobase.

27. The compound of claim 26, wherein C-(ZX⁴X⁵)s- comprises an anticancer agent selected from the group consisting of Taxol, Adriamycin, Dactinomycin, Bleomycin, Vinblastine and Cisplatin.

28. The compound of claim 26, wherein C-(ZX⁴X⁵)s- comprises a labeling agent selected from the group consisting of fluorescent labeling agents, quantum dots, magnetic resonance imaging (MRI) contrast agents, and radionuclides.

29. The compound of claim 26, wherein C-(ZX⁴X⁵)s- comprises an isotope selected from the group consisting of ^{99m}Tc, ¹¹¹In, ¹²³I, ¹³¹I, ⁶⁷Ga, ²⁰¹Tl, ¹²⁵I, ¹⁸F, ¹¹C, ⁷⁶Br, ¹²⁴I, ⁶⁸Ga, ⁸²Rb, ¹³N, ⁶⁴CU, ⁹⁰Y, ¹⁸⁸Rh, T (tritium), ³²P, ³⁵S, ¹⁵³Sm ⁸⁹Sr, and ²¹¹At.

30. The compound of claim 26, wherein C-(ZX⁴X⁵)s- comprises a fluorophore selected from the group consisting of ALEXA 350, PACIFIC BLUE, MARINA BLUE, ACRIDINE, EDANS, COUMARIN, BODIPY 493/503, CY2, BODIPY FL-X, DANSYL, ALEXA 488, FAM, OREGON GREEN, RHODAMINE GREEN-X, TET, ALEXA 430, CAL GOLD™, BODIPY R6G-X, JOE, ALEXA 532, VIC, HEX, CAL ORANGE™, ALEXA 555, BODIPY 564/570, BODIPY TMR-X, QUASAR™ 570, ALEXA 546, TAMRA, RHODAMINE RED-X, BODIPY 581/591, CY3.5, ROX, ALEXA 568, CAL RED™, BODIPY TR-X, ALEXA 594, BODIPY 630/650-X, PULSAR™ 650, BODIPY 630/665-X, ALEXA 647 and QUASAR™ 670.

31. The compound of claim 26, wherein at least one ZX⁴X⁵ comprises an optionally substituted adenine.

32. The compound of claim 31, wherein compound is represented by the following structural formula:

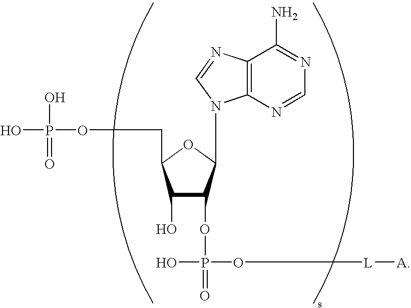

33. The compound of claim 31, wherein compound is represented by the following structural formula:

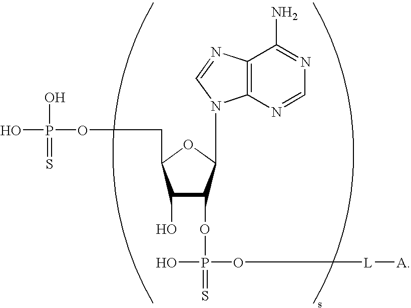

34. The compound of claim 32, wherein L comprises at least one ring selected from an optionally substituted 4 to 7 membered nonaromatic heterocyclic ring and an optionally substituted C4-C7 cycloalkyl ring.

35. The compound of claim 33, wherein L comprises at least one ring selected from an optionally substituted 4 to 7 membered nonaromatic heterocyclic ring and an optionally substituted C4-C7 cycloalkyl ring.

36. The compound of claim 35, wherein the compound is represented by the following structural formula:

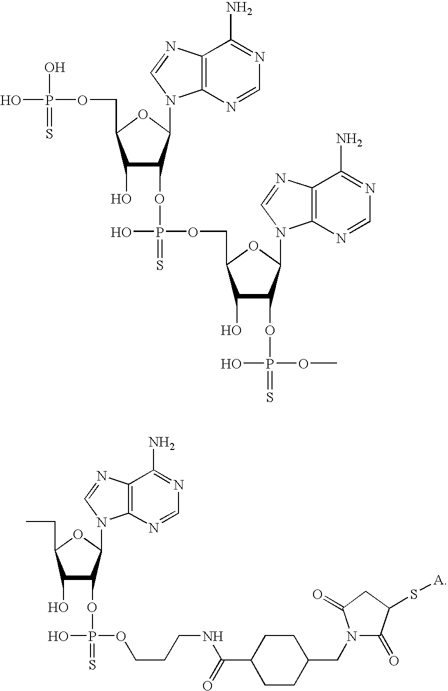

37. (canceled)

38. A pharmaceutical composition comprising a compound represented by the following structural formula:
C—B-L-A
or a pharmaceutically acceptable salt or solvate thereof, and a pharmaceutically acceptable carrier, wherein:
A is a prostate specific membrane antigen (PSMA) ligand;
L is an optionally substituted aliphatic or heteroaliphatic linking group;
B comprises at least one optionally substituted moiety selected from the group consisting of a sugar, a charged group, an aryl ring, and a heteroaryl ring, wherein B optionally comprises a drug or a labeling agent; and
C is H, a drug, or a labeling agent,
wherein CB together comprises the drug or the labeling agent.

39-42. (canceled)

43. A method of treating cancer, comprising administering to a subject in need thereof, a compound represented by the following structural formula:
C—B-L-A
or a pharmaceutically acceptable salt or solvate thereof, wherein:
A is a prostate specific membrane antigen (PSMA) ligand;
L is an optionally substituted aliphatic or heteroaliphatic linking group;
B comprises at least one optionally substituted moiety selected from the group consisting of a sugar, a charged group, an aryl ring, and a heteroaryl ring; wherein B is optionally a drug; and
C is H or a drug;
wherein CB together comprises the drug.

44-46. (canceled)

47. A method of inhibiting tumor neovascularization, comprising administering to a subject in need thereof, a compound represented by the following structural formula:
C—B-L-A
or a pharmaceutically acceptable salt or solvate thereof, wherein:
A is a prostate specific membrane antigen (PSMA) ligand;
L is an optionally substituted aliphatic or heteroaliphatic linking group;
B comprises at least one optionally substituted moiety selected from the group consisting of a sugar, a charged group, an aryl ring, and a heteroaryl ring; wherein B is optionally a drug; and
C is H or a drug;
wherein CB together comprises the drug.

48-49. (canceled)

50. A method of identifying a drug to treat cancer, comprising:
a) contacting a cell which expresses prostate specific membrane antigen with a compound represented by the following structural formula:
C—B-L-A
or a pharmaceutically acceptable salt or solvate thereof, wherein:
A is a prostate specific membrane antigen (PSMA) ligand;
L is an optionally substituted aliphatic or heteroaliphatic linking group;
B comprises at least one optionally substituted moiety selected from the group consisting of a sugar, a charged group, an aryl ring, and a heteroaryl ring; wherein B is optionally a drug; and
C is H or a drug;
wherein CB together comprises the drug; and
b) determining whether the compound has a therapeutic effect on the cell,
wherein if the compound has a therapeutic effect on the cell, then the compound can be used to treat cancer.

51-54. (canceled)

55. The method of claim 50, wherein the cancer is prostate cancer.

56. A method of identifying a drug that inhibits tumor neovascularization, comprising:
a) contacting a tumor neovasculature cell which expresses prostate specific membrane antigen with a compound represented by the following structural formula:
C—B-L-A
or a pharmaceutically acceptable salt or solvate thereof, wherein:
A is a prostate specific membrane antigen (PSMA) ligand;
L is an optionally substituted aliphatic or heteroaliphatic linking group;
B comprises at least one optionally substituted moiety selected from the group consisting of a sugar, a charged group, an aryl ring, and a heteroaryl ring; wherein B is optionally a drug; and
C is H or a drug;
wherein CB together comprises the drug; and
b) determining whether the compound has a therapeutic effect on the cell,
wherein if the compound has a therapeutic effect on the cell, then the compound can be used to inhibit tumor neovascularization.

57. A method of detecting cancer in a subject, comprising:
a) administering to the subject a compound represented by the following structural formula:
C—B-L-A
or a pharmaceutically acceptable salt or solvate thereof, wherein:
A is a prostate specific membrane antigen (PSMA) ligand;
L is an optionally substituted aliphatic or heteroaliphatic linking group;
B comprises at least one optionally substituted moiety selected from the group consisting of a sugar, a charged group, an aryl ring, and a heteroaryl ring; wherein B is optionally a labeling agent; and
C is H or a labeling agent;
wherein CB together comprises the labeling agent; and
b) detecting the labeling agent in the subject.

58-66. (canceled)

67. A method of identifying cancer cells in a sample, comprising:
a) contacting the sample with a compound represented by the following structural formula:
C—B-L-A
or a pharmaceutically acceptable salt or solvate thereof, wherein:
A is a prostate specific membrane antigen (PSMA) ligand;
L is an optionally substituted aliphatic or heteroaliphatic linking group;
B comprises at least one optionally substituted moiety selected from the group consisting of a sugar, a charged group, an aryl ring, and a heteroaryl ring; wherein B is optionally a labeling agent; and
C is H or a labeling agent;
wherein CB together comprises the labeling agent; and
b) detecting the labeling agent.

68-73. (canceled)

74. A kit, comprising a compound represented by the following structural formula:
C—B-L-A
or a pharmaceutically acceptable salt or solvate thereof, wherein:
A is a prostate specific membrane antigen (PSMA) ligand;
L is an optionally substituted aliphatic or heteroaliphatic linking group;
B comprises at least one optionally substituted moiety selected from the group consisting of a sugar, a charged group, an aryl ring, and a heteroaryl ring; wherein B is optionally a labeling agent;
C is H or a labeling agent; and
wherein CB together comprises the labeling agent,
provided that when A comprises HO₂CCH₂CH₂CH(CO₂H)CH₂—P(O)(OH)— or HO₂CCH₂CH₂CH(CO₂H)CH₂—OP(O)(OH)—, CB does not comprise: 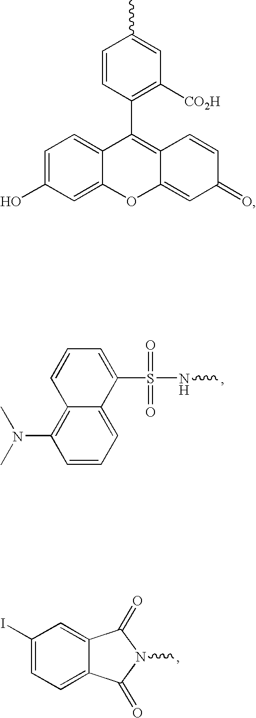
or unsubstituted

75. A method of treating a disease mediated by neovascularization, comprising administering to a subject in need thereof, a compound represented by the following structural formula:
C—B-L-A
or a pharmaceutically acceptable salt or solvate thereof, wherein:
A is a prostate specific membrane antigen (PSMA) ligand;
L is an optionally substituted aliphatic or heteroaliphatic linking group;
B comprises at least one optionally substituted moiety selected from the group consisting of a sugar, a charged group, an aryl ring, and a heteroaryl ring; wherein B is optionally a drug; and
C is H or a drug;
wherein CB together comprises the drug.

76-77. (canceled)

78. A method of treating a neurological disorder, comprising administering to a subject in need thereof, a compound represented by the following structural formula:
C—B-L-A
or a pharmaceutically acceptable salt or solvate thereof, wherein:
A is a prostate specific membrane antigen (PSMA) ligand;
L is an optionally substituted aliphatic or heteroaliphatic linking group;
B comprises at least one optionally substituted moiety selected from the group consisting of a sugar, a charged group, an aryl ring, and a heteroaryl ring; wherein B is optionally a drug; and
C is H or a drug;
wherein CB together comprises the drug.

79. (canceled)

80. The compound of claim 21, wherein the compound is represented by structural formula A2: 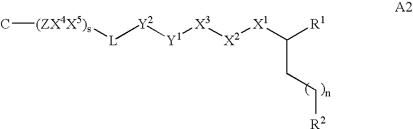
and pharmaceutically acceptable salts and solvates thereof, wherein:
n is 0, 1, or 2;
R¹ and R² are independently carboxylate or carboxylate bioisosteres;
X¹, X², X¹ and X⁴ are independently a bond, —NR^{a}—, —O—, —S—, —CR^{a}R^{b}—, —CR^{b}(OR^{a})—, —CR^{b}(SR^{a})—, —C(O)—, —C(S)—, —C(═CR^{a}R^{b})—, —C(═NR^{a})—, —C(═NOR^{a})—, —C(═NNR^{a})—, —S(O)—, —(SO₂)—, —S(O)(R^{a})—, —S(O)(OR^{a})—, —(PO₂)—, —P(O)(R^{a})—, —P(O)(OR^{a})—, —OP(O)(R^{a})—, —OP(O)(OR^{a})—, —P(S)(R^{a})—, —P(S)(OR^{a})—, —OP(S)(R^{a})—, or —OP(S)(OR^{a})—;
Y¹ and Y² are a bond, or Y¹ is an optionally substituted C1-C6 aliphatic chain and Y² is O or S;
L is an optionally substituted aliphatic or heteroaliphatic linking group;
s is an integer from 1 to 6, wherein the variables in each (ZX⁴X⁵) are independently selected;
each Z is independently an optionally substituted aryl, heteroaryl, cycloaliphatic, or non-aromatic heterocyclic group, provided that at least one Z comprises an aryl, heteroaryl, nucleobase, nucleoside, or nucleotide;
X⁵ is a bond or methylene;
C is H, a drug, or a labeling agent, whereby C-(ZX⁴X⁵)s- comprises a drug or a labeling agent; and
R^{a} and R^{b} are each independently —H or an optionally substituted aliphatic, optionally substituted cycloaliphatic, optionally substituted heterocyclic, optionally substituted benzyl, optionally substituted aryl, or optionally substituted heteroaryl,
provided that when A2 comprises HO₂CCH₂CH₂CH(CO₂H)CH₂—P(O)(OH)— or HO₂CCH₂CH₂CH(CO₂H)CH₂—OP(O)(OH)—, C-(ZX⁴X⁵)s- does not comprise: 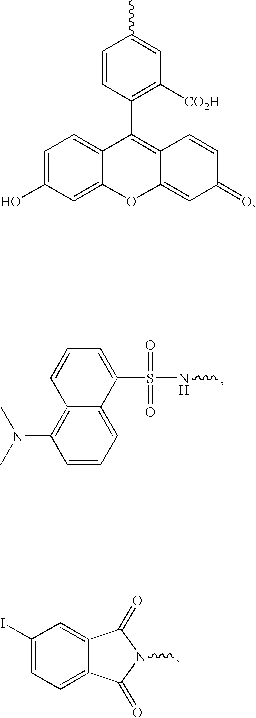
or unsubstituted phenyl;
when A2 comprises a moiety represented by either of the following structural formulas: 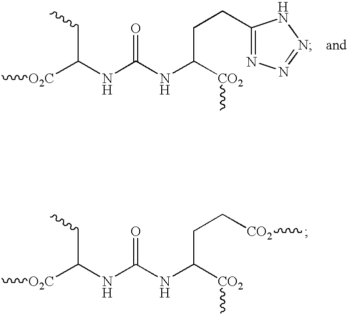
C-(ZX⁴X⁵)s- comprises at least one group selected from: a covalently attached, nonmetallic charged group other than carboxylate or protonated amine; a sugar; and a heteroaryl or non-aromatic heterocycle having at least two heteroatoms;
when A2 comprises a moiety represented by the following structural formula: 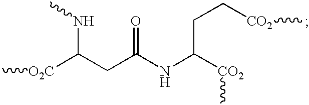
C-(ZX⁴X⁵)s- comprises at least one group selected from: a covalently attached, nonmetallic charged group other than carboxylate, protonated amine, or sulfate; a sugar; and a heteroaryl or non-aromatic heterocycle having at least two heteroatoms; and
when A1 includes a moiety represented by the following structural formula: 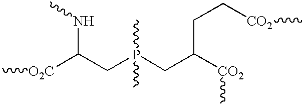
C-(ZX⁴X⁵)s- comprises at least one group selected from: a covalently attached, nonmetallic charged group other than carboxylate or protonated amine; a sugar other than an aminosaccharide; and a heteroaryl or non-aromatic heterocycle.

81. The compound of claim 80, wherein the drug or the labeling agent is coupled to the rest of the compound by a cleavable linker.

82. The compound of claim 81, wherein optional substituents are independently selected from the group consisting of —F, —Cl, —Br, —I, —CN, —NO2, —OR^{a}, —C(O)R^{a}, —OC(O)R^{a}, —C(O)OR^{a}, —SR^{a}, —C(S)R^{a}, —OC(S)R^{b}, —C(S)OR^{a}, —C(O)SR^{a}, —C(S)SR^{a}, S(O)R^{a}—SO₂R^{a}, —SO₃R^{a}, —POR^{a}R^{b}, —PO₂R^{a}R^{b}, —PO₃R^{a}R^{b}, —PO₄R^{a}R^{b}, —P(S)R^{a}R^{b}, —P(S)OR^{a}R^{b}, —P(S)O₂R^{a}R^{b}, —P(S)O₃R^{a}R^{b}, —N(R^{a}R^{b}), C(O)N(R^{a}R^{b}), —C(O)NR^{a}NR^{b}SO₂R^{c}, —C(O)NR^{a}SO₂R^{c}C₁—C(O)NR^{a}CN, —SO₂N(R^{a}R^{b}), —SO₂N(R^{a}R^{b}), —NR^{c}C(O)R^{a}, NR^{c}C(O)OR^{a}, —NR^{c}C(O)N(R^{a}R^{b}), —C(NR^{c})—N(R^{a}R^{b}), —NR^{d}—C(NR^{c})—N(R^{a}R^{b}), NR^{a}N(R^{a}R^{a}), —CR^{c}═CR^{a}R^{b}, —C═CR^{a}, ═O, ═S, ═CR^{a}R^{b}, ═NR^{a}, ═NOR^{a}, ═NNR^{a}, optionally substituted alkyl, optionally substituted cycloalkyl, optionally substituted aliphatic, optionally substituted cycloaliphatic, optionally substituted heterocyclic, optionally substituted benzyl, optionally substituted aryl, and optionally substituted heteroaryl; wherein R^{a}-R^{d} are each independently —H or an optionally substituted aliphatic, optionally substituted cycloaliphatic, optionally substituted heterocyclic, optionally substituted benzyl, optionally substituted aryl, or optionally substituted heteroaryl, or, N(R^{a}R^{b}), taken together, is an optionally substituted heterocyclic group.

83. The compound of claim 82, wherein R¹ and R² are independently —OH, -phenol, —C(O)OH, —C(S)OH, —C(O)SH, —C(S)SH, —SO₂H, —SO₃H, —PO₂H₂, —PO₃H₂, —NHR^{a}, —NH—, C(O)NHR^{a}, —C(O)NHSO₂R^{c}, —C(O)NHS₂R^{c}, —SO₂NHR^{a}, —SO₂NHR^{a}, —NHC(O)R^{a}, NHC(O)OR^{a}, —NHC(O)NHR^{a}═NH, or optionally substituted tetrazole, 1,2,3-triazole, 1,2,4-triazole or imidazole.

84. The compound of claim 83, wherein C—(ZX⁴X⁵)s- comprises an anticancer agent selected from the group consisting of Taxol, Adriamycin, Dactinomycin, Bleomycin, Vinblastine and Cisplatin.

85. The compound of claim 83, wherein C-(ZX⁴X⁵)s- comprises a labeling agent selected from the group consisting of fluorescent labeling agents, quantum dots, magnetic resonance imaging (MRI) contrast agents, and radionuclides.

86. The compound of claim 83, wherein C-(ZX⁴X⁵)s- comprises an isotope selected from the group consisting of ^{99m}Tc, ¹¹¹In, ¹²³I, ¹³¹I, ⁶⁷Ga, ²⁰¹Tl, ¹²⁵I, ¹⁸F, ¹¹C, ⁷⁶Br, ¹²⁴I, ⁶⁸Ga, ⁸²Rb, ¹³N, ⁶⁴Cu, ⁹⁰Y, ¹⁸⁸Rh, T (tritium), ³²P, ³⁵S, ¹⁵³Sm, ⁸⁹Sr, and ²¹¹At.

87. The compound of claim 83, wherein C-(ZX⁴X⁵)s- comprises a fluorophore selected from the group consisting of ALEXA 350, PACIFIC BLUE, MARINA BLUE, ACRIDINE, EDANS, COUMARIN, BODIPY 493/503, CY2, BODIPY FL-X, DANSYL, ALEXA 488, FAM, OREGON GREEN, RHODAMINE GREEN-X, TET, ALEXA 430, CAL GOLD™, BODIPY R6G-X, JOE, ALEXA 532, VIC, HEX, CAL ORANGE™, ALEXA 555, BODIPY 564/570, BODIPY TMR-X, QUASAR™ 570, ALEXA 546, TAMRA, RHODAMINE RED-X, BODIPY 581/591, CY3.5, ROX, ALEXA 568, CAL RED™, BODIPY TR-X, ALEXA 594, BODIPY 630/650-X, PULSAR™ 650, BODIPY 630/665-X, ALEXA 647 and QUASAR™ 670.

88. The compound of claim 83, wherein the compound is represented by the following structural formula:

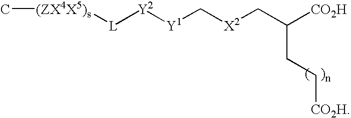

89. The compound of claim 88, wherein X² is —(PO₂)—, —P(O)(R^{a})—, —P(O)(OR^{a})—, —OP(O)(R^{a})—, —OP(O)(OR^{a})—, —P(S)(R^{a})—, —P(S)(OR^{a})—, —OP(S)(R^{a})—, or —OP(S)(OR^{a})—.

90. The compound of claim 83, wherein the compound is represented by the following structural formula:

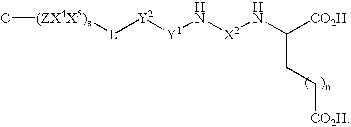

91. The compound of claim 90, wherein the compound is represented by the following structural formula: 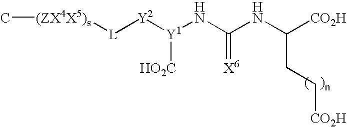
wherein X⁶ is O or S.

92. The compound of claim 91, wherein the compound is represented by the following structural formula:

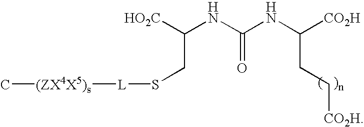

93. The compound of claim 92, wherein each ZX⁴X⁵ comprises an optionally substituted nucleobase.

94. The compound of claim 93, wherein at least one ZX⁴X⁵ comprises an optionally substituted adenine.

95. The compound of claim 94, wherein the compound is represented by the following structural formula:

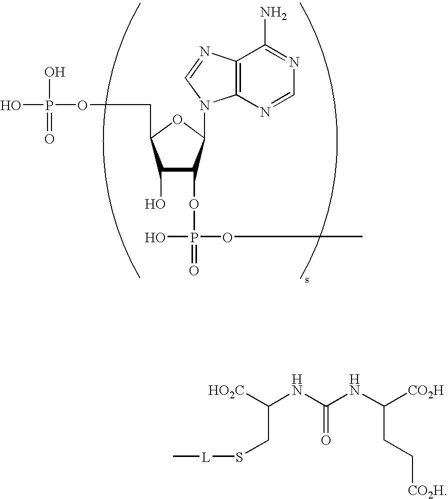

96. The compound of claim 95, wherein the compound is represented by the following structural formula:

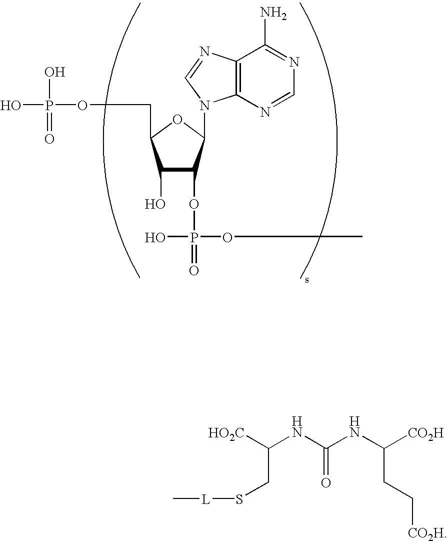

97. The compound of claim 95, wherein L comprises at least one ring selected from an optionally substituted 4 to 7 membered nonaromatic heterocyclic ring and an optionally substituted C4-C7 cycloalkyl ring.

98. The compound of claim 96, wherein L comprises at least one ring selected from an optionally substituted 4 to 7 membered nonaromatic heterocyclic ring and an optionally substituted C4-C7 cycloalkyl ring.

99. The compound of claim 98, wherein the compound is:

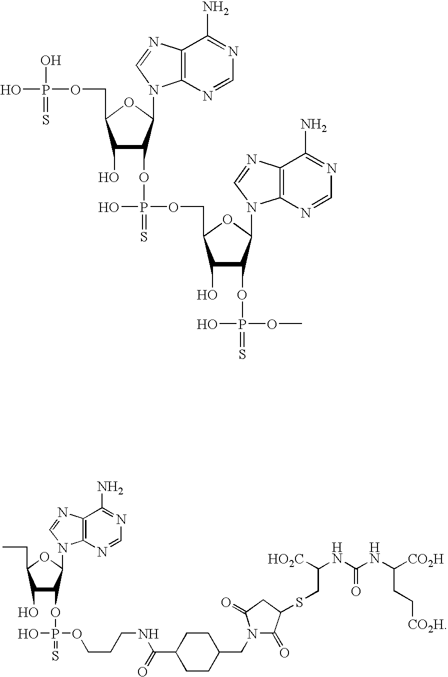

100. The composition of claim 38, wherein the compound is represented by the following structural formula:
C-(ZX⁴X⁵)ₛ-L-A
wherein:
X⁴ is a bond, —NR^{a}, —O—, —S—, —CR^{a}R^{b}—, —CR^{b}(OR^{a})—, —CR^{b}(SR^{a})—, —C(O)—, —C(S)—, —C(═CR^{a}R^{b})—, —C(═NR^{a})—, —C(═NOR^{a})—, —C(═NNR^{a})—, —S(O)—, —(SO₂)—, —S(O)(R^{a})—, —S(O)(OR^{a})—, —(PO₂)—, —P(O)(R^{a})—, —P(O)(OR^{a})—, —OP(O)(R^{a})—, —OP(O)(OR^{a})—, —P(S)(R^{a})—, —P(S)(OR^{a})—, —OP(S)(R^{a})—, or —OP(S)(OR^{a})—;
L is an optionally substituted aliphatic or heteroaliphatic linking group;
s is an integer from 1 to 6, wherein the variables in each (ZX⁴X⁵) are independently selected;
each Z is independently an optionally substituted aryl, heteroaryl, cycloaliphatic, or non-aromatic heterocyclic group, provided that at least one Z is an aryl or heteroaryl group or is substituted with an aryl or heteroaryl group; and
X⁵ is a bond or methylene,
wherein R^{a} and R^{b} are each independently —H or an optionally substituted aliphatic, optionally substituted cycloaliphatic, optionally substituted heterocyclic, optionally substituted benzyl, optionally substituted aryl, or optionally substituted heteroaryl.

101. The composition of claim 38, wherein the compound is represented by the following structural formula: 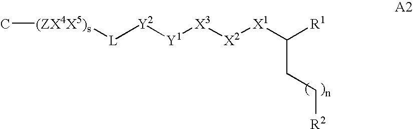
and pharmaceutically acceptable salts and solvates thereof, wherein:
n is 0, 1, or 2;
R¹ and R² are independently carboxylate or carboxylate bioisosteres;
X¹, X², X¹ and X⁴ are independently a bond, —NR^{a}—, —O—, —S—, —CR^{a}R^{b}—, —CR^{b}(OR^{a})—, —CR^{b}(SR^{a})—, —C(O)—, —C(S)—, —C(═CR^{a}R^{b})—, —C(═NR^{a})—, —C(═NOR^{a})—, —C(═NNR^{a})—, —S(O)—, —(SO₂)—, —S(O)(R^{a})—, —S(O)(OR^{a})—, —(PO₂)—, —P(O)(R^{a})—, —P(O)(OR^{a})—, —OP(O)(R^{a})—, —OP(O)(OR^{a})—, —P(S)(R^{a})—, —P(S)(OR^{a})—, —OP(S)(R^{a})—, or —OP(S)(OR^{a})—;
Y¹ and Y² are a bond, or Y¹ is an optionally substituted C1-C6 aliphatic chain and Y² is O or S;
L is an optionally substituted aliphatic or heteroaliphatic linking group;
s is an integer from 1 to 6, wherein the variables in each (ZX⁴X⁵) are independently selected;
each Z is independently an optionally substituted aryl, heteroaryl, cycloaliphatic, or non-aromatic heterocyclic group, provided that at least one Z comprises an aryl, heteroaryl, nucleobase, nucleoside, or nucleotide;
X⁵ is a bond or methylene;
C is H, a drug, or a labeling agent, whereby C-(ZX⁴X⁵)s- comprises a drug or a labeling agent; and
R^{a} and R^{b} are each independently —H or an optionally substituted aliphatic, optionally substituted cycloaliphatic, optionally substituted heterocyclic, optionally substituted benzyl, optionally substituted aryl, or optionally substituted heteroaryl,
provided that when A2 comprises HO₂CCH₂CH₂CH(CO₂H)CH₂—P(O)(OH)— or HO₂CCH₂CH₂CH(CO₂H)CH₂—OP(O)(OH)—, C-(ZX⁴X⁵)s- does not comprise: 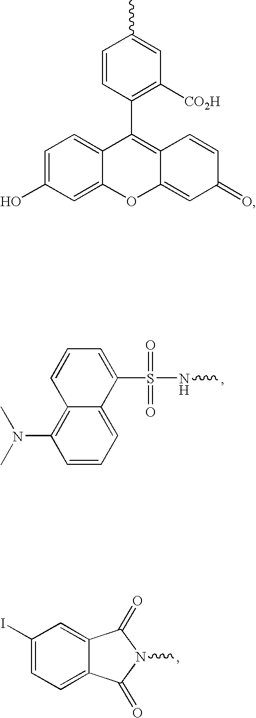
or unsubstituted phenyl;
when A2 comprises a moiety represented by either of the following structural formulas: 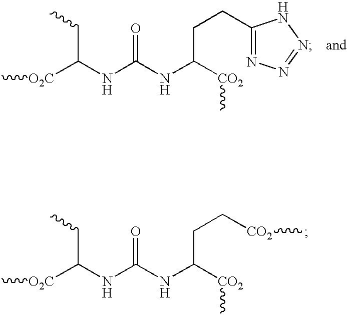
C-(ZX⁴X⁵)s- comprises at least one group selected from: a covalently attached, nonmetallic charged group other than carboxylate or protonated amine; a sugar; and a heteroaryl or non-aromatic heterocycle having at least two heteroatoms;
when A2 comprises a moiety represented by the following structural formula: 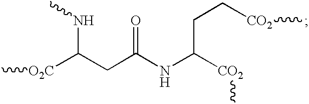
C-(ZX⁴X⁵)s- comprises at least one group selected from: a covalently attached, nonmetallic charged group other than carboxylate, protonated amine, or sulfate; a sugar; and a heteroaryl or non-aromatic heterocycle having at least two heteroatoms; and
when A1 includes a moiety represented by the following structural formula: 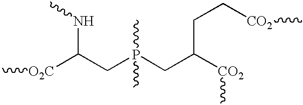
C-(ZX⁴X⁵)s- comprises at least one group selected from: a covalently attached, nonmetallic charged group other than carboxylate or protonated amine; a sugar other than an aminosaccharide; and a heteroaryl or non-aromatic heterocycle.

102. The composition of claim 101, wherein the compound is represented by the structure formula: [chemical structure]
